SEQUENCE LISTING (26 sequences)

SEQ ID NO: 1 - DNA, 19 bp, human
  primer_bind (1)..(19): GD2 synthase sense primer
gacaagccagagcgcgtta

SEQ ID NO: 2 - DNA, 21 bp, human
  primer_bind (1)..(21): GD2 synthase antisense primer
tacttgagacacggccaggtt

SEQ ID NO: 3 - DNA, 21 bp, artificial sequence
  GD2 synthase probe
aaccagcccttgccgaagggc

SEQ ID NO: 4 - DNA, 19 bp, human
  primer_bind (1)..(19): GAPDH sense primer
gaaggtgaaggtcggagtc

SEQ ID NO: 5 - DNA, 20 bp, human
  primer_bind (1)..(20): GAPDH antisense primer
gaagatggtgatgggatttc

SEQ ID NO: 6 - DNA, 20 bp, artificial sequence
  GAPDH probe
caagcttcccgttctcagcc

SEQ ID NO: 7 - DNA, 20 bp, human
  primer_bind (1)..(20): Oligonucleotides used as PCR primers for detecting GD2 synthase-sense primer
ccaactcaacaggcaactac

SEQ ID NO: 8 - DNA, 20 bp, human
  primer_bind (1)..(20): Oligonucleotides used as PCR primers for detecting GD2 synthase-antisense primer
gatcataacggaggaaggtc

SEQ ID NO: 9 - DNA, 23 bp, human
  primer_bind (1)..(23): Primers specific for human B2-microglobulin (a)
ctcgcgctactctctctttctgg

SEQ ID NO: 10 - DNA, 25 bp, human
  primer_bind (1)..(25): Primers specific for human B2-microglobulin (b)
gcttacatgtctcgatcccacttaa

SEQ ID NO: 11 - DNA, 18 bp, human
  primer_bind (1)..(18): GAGE1-specific forward primer
agacgctacgtagcgcct

SEQ ID NO: 12 - DNA, 18 bp, human
  primer_bind (1)..(18): GAGE1-specific reverse primer
ccatcaggaccatcttca

SEQ ID NO: 13 - DNA, 18 bp, artificial sequence
  GAGE1-specific probe that incorporated digoxigenin-11-UTP-forward primer
cgagcagttcagtgatga

SEQ ID NO: 14 - DNA, 18 bp, artificial sequence
  GAGE1-specific probe that incorporated digoxigenin-11-UTP-reverse primer
accctgttcctggctatc

SEQ ID NO: 15 - DNA, 23 bp, artificial sequence
  BAGE probe
agaaaaaccgctctggccgccat

SEQ ID NO: 16 - DNA, 20 bp, human
  primer_bind (1)..(20): BAGE forward primer
gatggtggtggcaacagaga

SEQ ID NO: 17 - DNA, 19 bp, human
  primer_bind (1)..(19): BAGE reverse primer
ttcatcagcctggcttgga

SEQ ID NO: 18 - DNA, 25 bp, artificial sequence
  WT-1 probe
ctcgtaccctgtgctgtggcccttt

SEQ ID NO: 19 - DNA, 20 bp, human
  primer_bind (1)..(20): WT-1 forward primer
tcccagcttgaatgcatgac

SEQ ID NO: 20 - DNA, 21 bp, human
  primer_bind (1)..(21): WT-1 reverse primer
gatgggcgttgtgtggttatc

SEQ ID NO: 21 - DNA, 28 bp, artificial sequence
  GAGE1 probe
ttgcccttcacatgccacagatgatagg

SEQ ID NO: 22 - DNA, 20 bp, human
  primer_bind (1)..(20): GAGE1 forward primer
caaatggcgagagaccgttt

SEQ ID NO: 23 - DNA, 20 bp, human
  primer_bind (1)..(20): GAGE1 reverse primer
cataggagcagcctgcaaca

SEQ ID NO: 24 - DNA, 26 bp, artificial sequence
  MIDKINE probe
ttccctttcttggctttggcctttgc

SEQ ID NO: 25 - DNA, 20 bp, human
  primer_bind (1)..(20): MIDKINE forward primer
gctcagtgccaggagaccat

SEQ ID NO: 26 - DNA, 20 bp, human
  primer_bind (1)..(20): MIDKINE reverse primer
tccaggcttggcgtctagtc

(19) United States
(12) Patent Application Publication
(10) Pub. No.: US 2004/0115688 A1
(43) Pub. Date: Jun. 17, 2004

(54) Detection of gd2 synthase mrna and uses thereof

(76) Inventors: Irene Y. Cheung, Purchase, NY (US); Nai-King V Cheung, Purchase (GB)

Correspondence Address: Albert Wai-Kit Chan, Law Offices of Albert Wai-Kit Chan, World Plaza Suite 604, ,141-07 20th Avene, Whitestone, NY, 11357, US

(21) Appl. No.: 10/477,435

(22) PCT Filed: Apr. 19, 2002
(86) PCT No.: PCT/US02/15037

(51) Int. Cl. C12Q 1/68

(52) U.S. Cl. 435/6

(57) ABSTRACT

The present invention provides a method to measure GD2 synthase mRNA comprising steps of: (a) obtaining a mRNA sample; (b) performing real-time quantitative RT-PCR on the sample using appropriate primers of GD2 synthase; and (c) determining the amount of GD2 synthase mRNA. The invention also provides a method to diagnose a subject which bears cancer expressing GD2 synthase. Furthermore, this invention provides a method to stage a cancer expressing GD2 synthase in a subject. Finally, this invention provides a kit for detection of GD2 synthase.

This application claims priority of U.S. Ser. No. 60/290,527, filed 11 May 2001, the content of which is incorporated by reference here into this application.

The invention disclosed herein was made with government support under NIH Grant No. CA61017, from the United States Department of Health and Human Services. Accordingly, the U.S. Government has certain rights in this invention.

Throughout this application, various references are referred to. Disclosures of these publications in their entireties are hereby incorporated by reference into this application to more fully describe the state of the art to which this invention pertains.

### BACKGROUND OF THE INVENTION

As the thoroughfare for cellular trafficking, blood and bone marrow (BM) harbor tumor micrometastases that seed organs distant from the primary site. The presence of these occult tumor cells has grave prognostic significance in various malignancies, including breast (1), colon (2), stomach (3), lung (4), melanoma (5), and leukemia (6). For the childhood cancer neuroblastoma (NB), BM disease is associated with an unfavorable outcome (7, 8). Histological examinations by Hematoxylin-Eosin stain of marrow biopsies and Wright-Giemsa stain of marrow aspirates have been the standard techniques to monitor BM disease. However, this can grossly underestimate its prevalence in neuroblastoma, even if multiple sites are tested (9). Because of its superior sensitivity, immunocytology using monoclonal antibodies can complement and augment morphologic methods of tumor detection (9, 10). The use of molecular markers of NB has also been explored to detect BM disease, in particular, the lineage-specific gene transcript tyrosine hydroxylase, which is the first and rate-limiting enzyme in the catecholamine biosynthesis pathway. It is useful in detecting tumor cells in the BM and blood (11). However, its expression may be down-regulated in some cells (12). More recently, cancer-testis antigen GAGE1 is found to be comparable in sensitivity to immunocytology in detecting neuroblastoma cells in the bone marrow (13). Molecular detection by RT-PCR has thus far been qualitative. It was reasoned that quantitative PCR measurements of tumor cells over the course of treatment in the marrow and blood can provide substantially more information, allowing comparisons to be made between treatment phases and approaches, as well as among patients.

The emergence of real-time quantitative PCR technology has facilitated the evaluation of microscopic tumors. PCR detection of clonal genomic immunoglobulin H gene rearrangement in B cell malignancies, including multiple myelomas (14), acute lymphoblastic leukemia (15), and chronic myelogenous leukemia (16) have redefined the meaning of remission that will likely change the way these diseases are managed. In addition, real-time quantitative PCR has been used successfully in measuring EBV-viral DNA, a powerful predictor of tumor recurrence in nasopharyngeal carcinoma (17). It compares favorably with nested competitive RT-PCR in sensitivity, linearity, and reproducibility (18). In NB, the choice of GD2 synthase for real-time quantitative RT-PCR assay is particularly attractive, since GD2 is homogeneously expressed in NB of all stages. GD2 density on neuroblastoma cells is high (5-10×10⁶ molecules per cell) (19) and this glycosphingolipid antigen is rarely lost after anti-GD2 therapy (20). GD2 synthesis is dependent on a key enzyme β1,4-N-acetylgalactosaminyltransferase (GD2/GM2 synthase) which catalyzes the transfer of β1,4-N-acetylgalactosamine to the precursor gangliosides GD3/GM3, respectively (21). In this report, the enzyme will be termed GD2 synthase for clarity, and not GD2/GM2 synthase. Its gene transcript level was found to correlate with the enzyme activity, as well as GD2 expression in individual cell lines (22). By competitive RT-PCR, GD2 synthase mRNA expression was enhanced in some gastric and colon carcinomas, when compared to normal mucosa (23).

It is hypothesized that GD2 synthase mRNA may potentially be a useful molecular marker for the detection of NB in the BM or blood. To date, there has been only one report on the detection of GD2 synthase transcript by RT-PCR and Southern blot analysis of melanoma cell lines, and in the blood of some patients with advanced stages of malignant melanoma (24). The objective of this study is to measure GD2 synthase mRNA by real-time quantitative RT-PCR, and to use it as a marker of tumor cells in the BM of NB patients. The quantitative relationship between transcription level and the percent of GD2-positive tumor cells as enumerated by immunocytology is to be determined. The clinical significance of marrow GD2 synthase will be tested among patients in clinical remission. The serial levels of transcript in individual patients with their clinical status will also be correlated.

### SUMMARY OF THE INVENTION

The present invention provides a method to measure GD2 synthase mRNA comprising steps of: (a) obtaining a mRNA sample; (b) performing real-time quantitative RT-PCR on the sample using appropriate primers of GD2 synthase; and (c) determining the amount of GD2 synthase mRNA. The invention also provides a method to diagnose a subject which bears cancer expressing GD2 synthase comprising steps of: (a) obtaining a mRNA sample from the subject; (b) performing RT-PCR on the sample using appropriate primers of GD2 synthase; and (c) determining the amount of GD2 synthase mRNA. Furthermore, this invention provides a method to stage a cancer expressing GD2 synthase in a subject comprising steps of: (a) obtaining a mRNA sample from the subject known to carry the disease at different stages; (b) performing real-time quantitative RT-PCR on the sample obtained from step (a) using appropriate primers of GD2 synthase; (c) determining the amount of the GD2 synthase mRNA; (d) comparing the amount obtained in step (c) from the different stages to obtain a standard curve; (e) obtaining a mRNA sample from a test subject with the cancer; (f) determining the amount of mRNA from the sample using real-time quantitative RT-PCR; (g) comparing the amount determined in step (f) with the standard curve obtained in step (d), thereby determining the stage of the cancer in the test subject.

### DETAILED DESCRIPTION OF THE FIGURES

First Series of Experiments

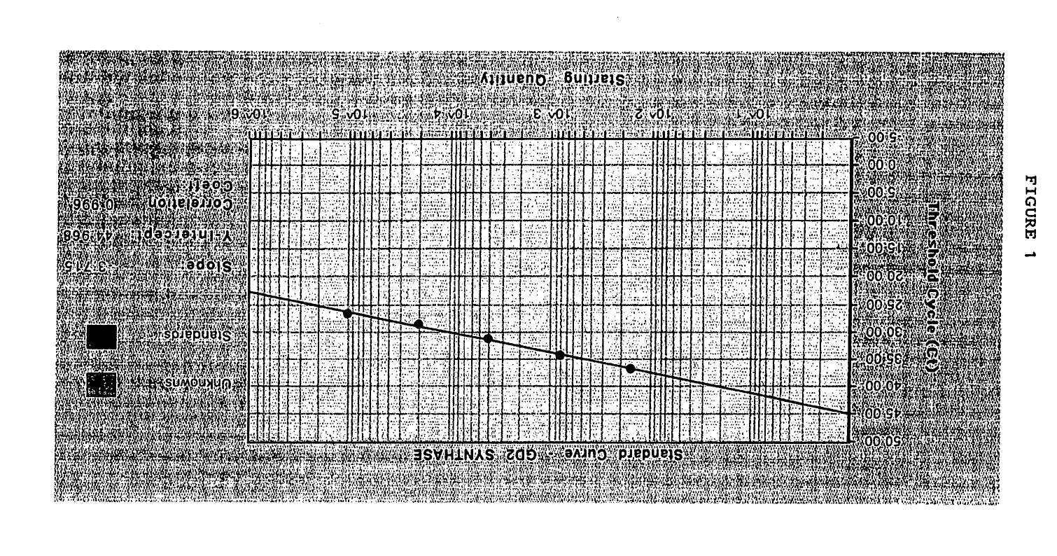

FIG. 1. A representative standard curve of real-time quantitative RT-PCR of GD2 synthase mRNA was derived from serially diluted cDNAs of a NB cell line NMB7.

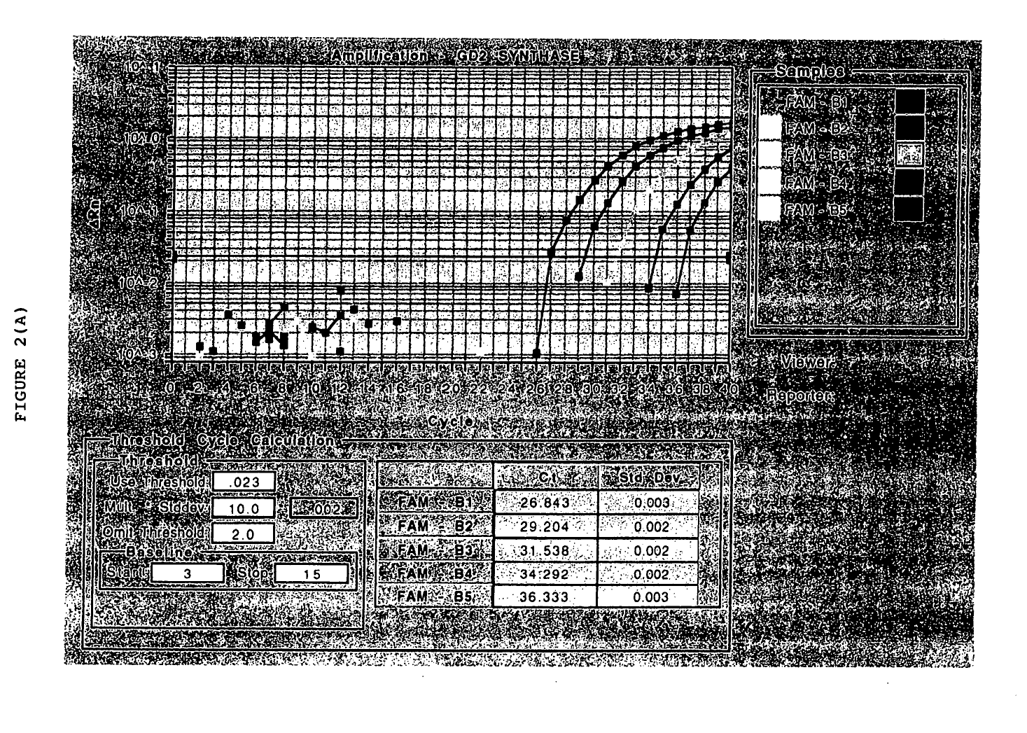

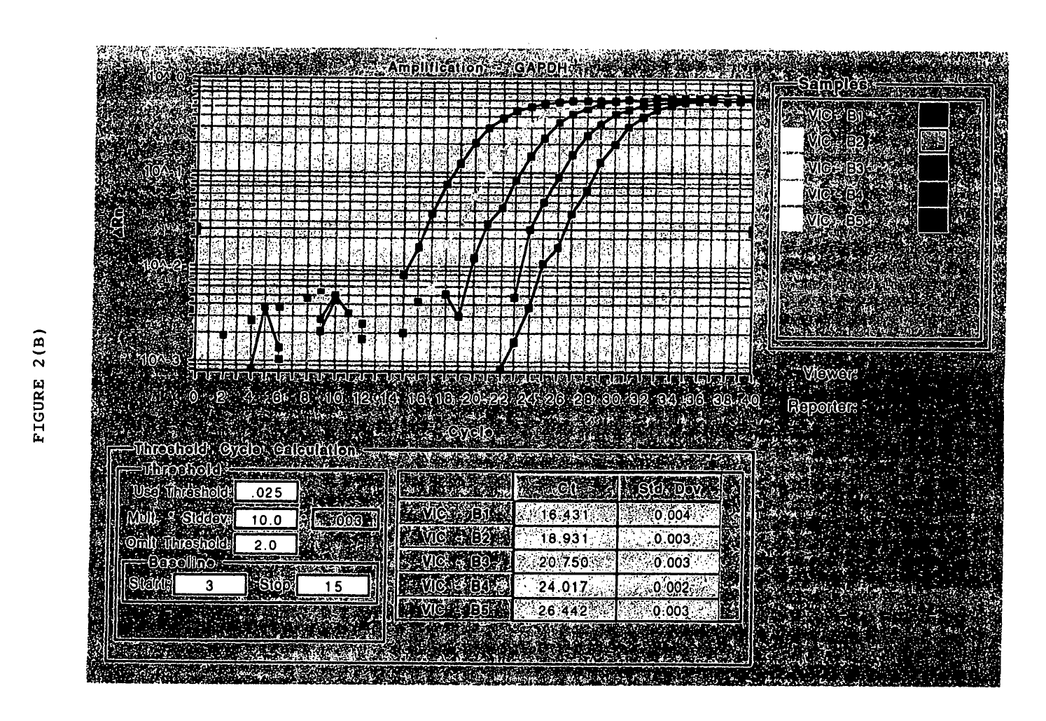

FIG. 2. Amplification plot of GD2 synthase (2A) and GAPDH (2B) standard curve.

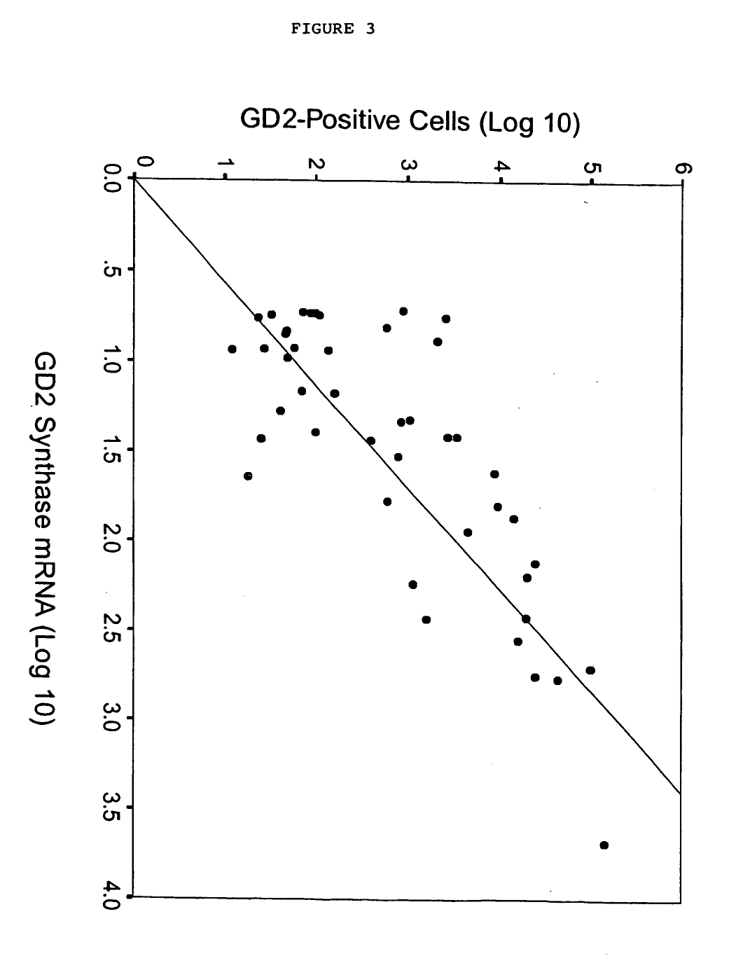

FIG. 3. Correlation between quantitation by immunocytology and GD2 synthase mRNA. Both axes were log (10). Immunocytology was expressed in number of GD2 positive cells per 10⁶ marrow mononuclear cells. GD2 synthase mRNA was measured as described in Patients and Methods section. Correlation coefficient=0.96.

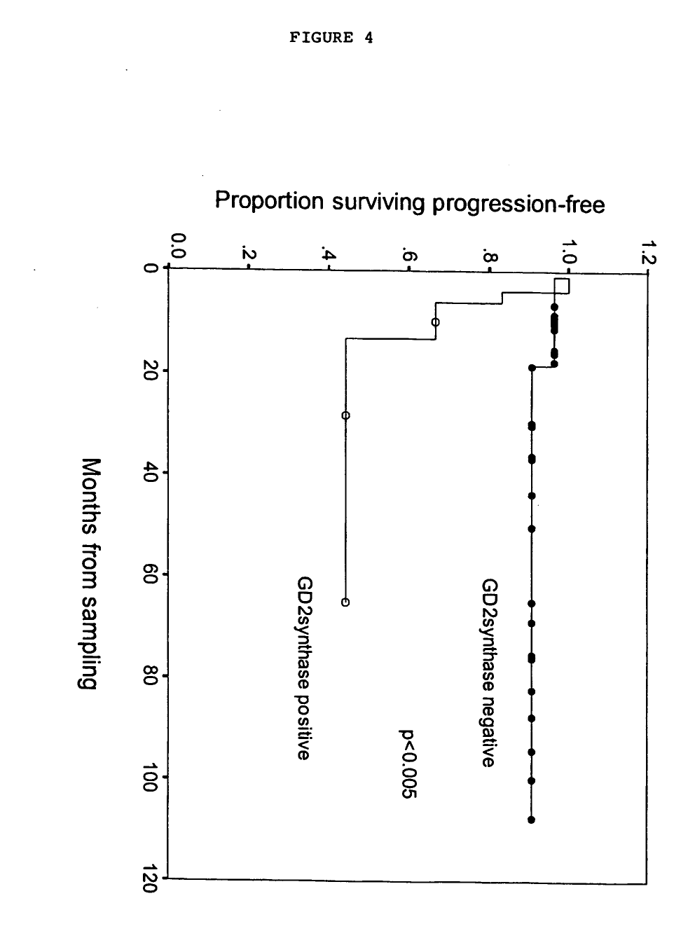

FIG. 4. Relationship between GD2 synthase mRNA and Kaplan-Meier analysis of progression-free survival from remission marrows sampled at 24 months from diagnosis.

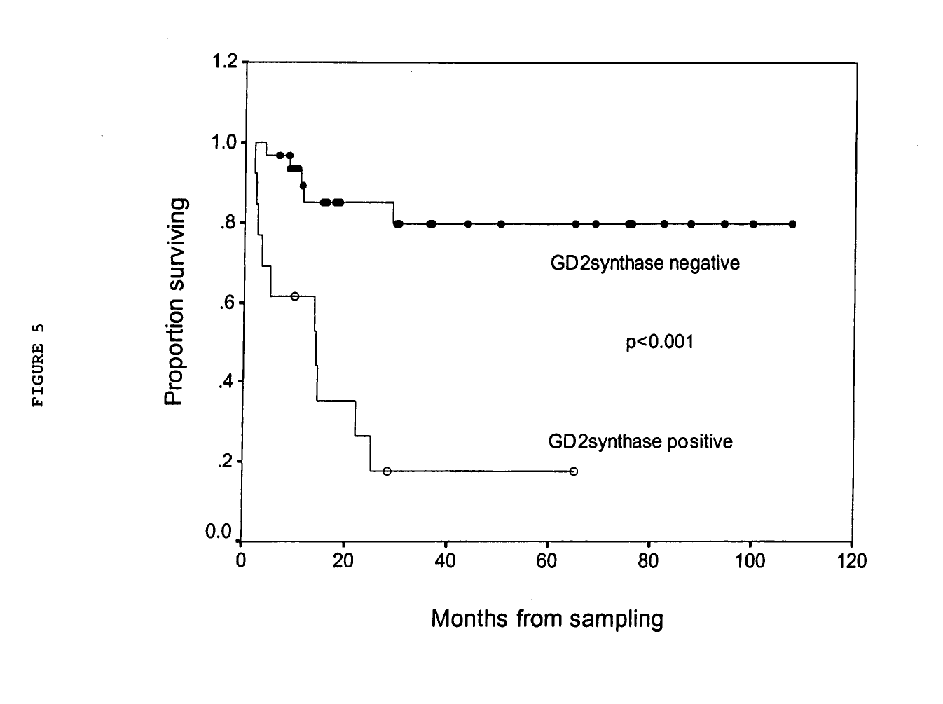

FIG. 5. Relationship between GD2 synthase mRNA and Kaplan-Meier analysis of survival from remission marrows sampled at 24 months from diagnosis.

Second Series of Experiments

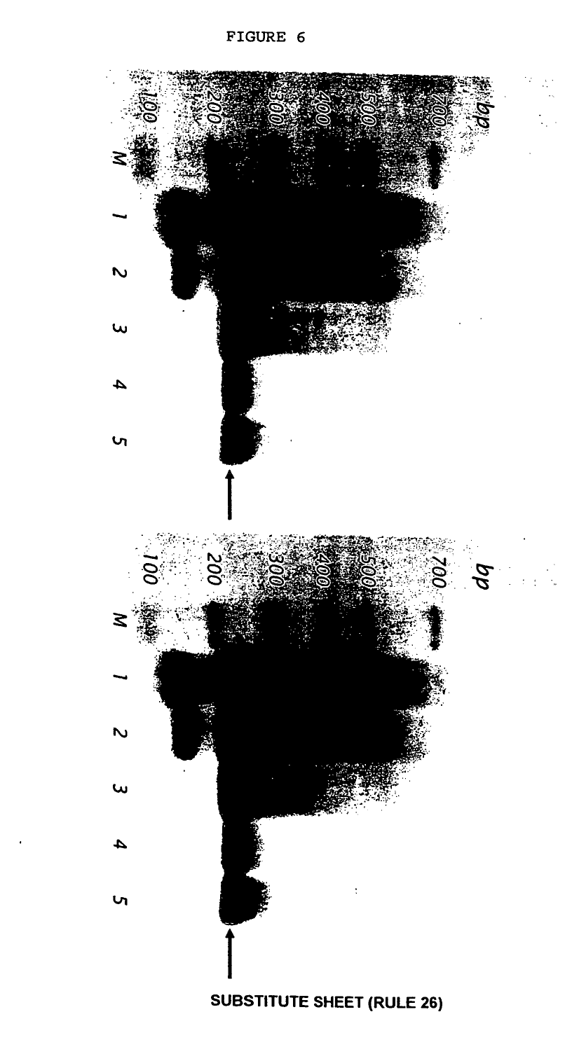

FIG. 6. Sensitivity of GD2 synthase RT-PCR. mRNAs were extracted from 10⁶ marrow cells seeded with LAN-1 NB cells at concentration of 10⁴ (lane 1), 10³ (lane 2), 10² (lane 3), 10 (lane 4), and 1 (lane 5). After RT-PCR, GD2 synthase transcript was detected by chemiluminescence. Lane M, molecular weight marker. Arrow, 230 bp.

Third Series of Experiments

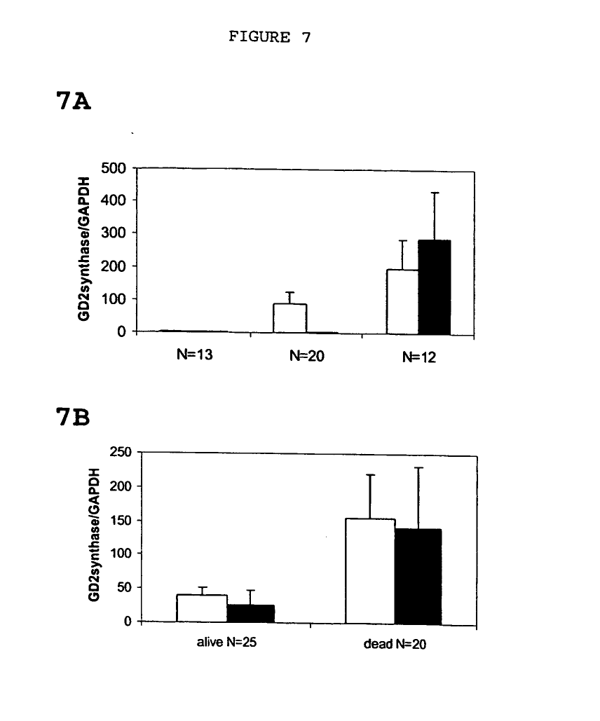

FIG. 7. Levels of GD2 synthase transcript (7A) among patients who had no detectable transcript before (open bar) and after (solid bar) 3F8-targeted treatment, versus those whose marrows became negative after treatment, versus those whose marrows remained positive; (7B) Patients who remain alive versus those who died.

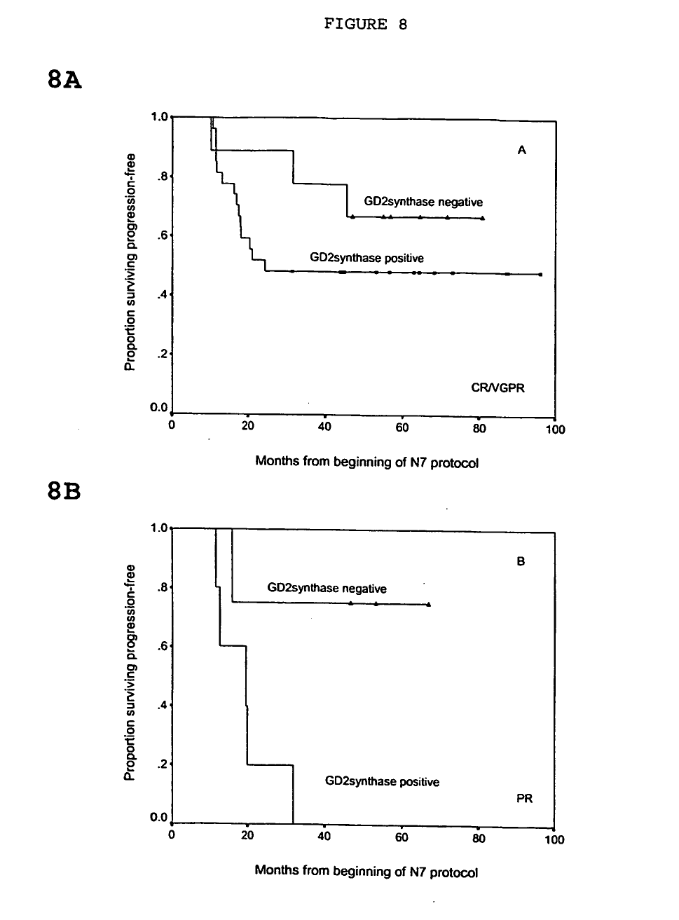

FIG. 8. Kaplan-Meier plots of progression-free survival based on the detection of GD2 synthase mRNA prior to ¹³¹I-3F8 radioimmunotherapy of 45 stage 4 NB patients (newly diagnosed/without prior relapse) stratified by (8A) complete remission (CR)/very good partial remission (VGPR), (8B) partial remission (PR). P=0.045

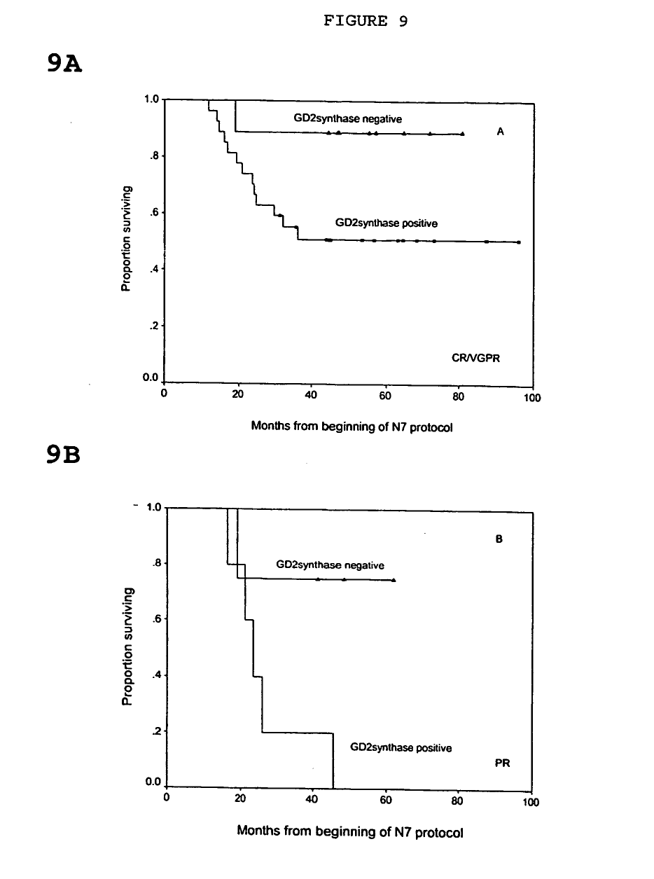

FIG. 9. Kaplan-Meier plots of overall survival based on detection of GD2 synthase mRNA prior to ¹³¹I-3F8 radioimmunotherapy of 45 stage 4 NB patients (newly diagnosed/without prior relapse) stratified by (9A) complete remission (CR)/very good partial remission (VGPR), (9B) partial remission (PR). P=0.010

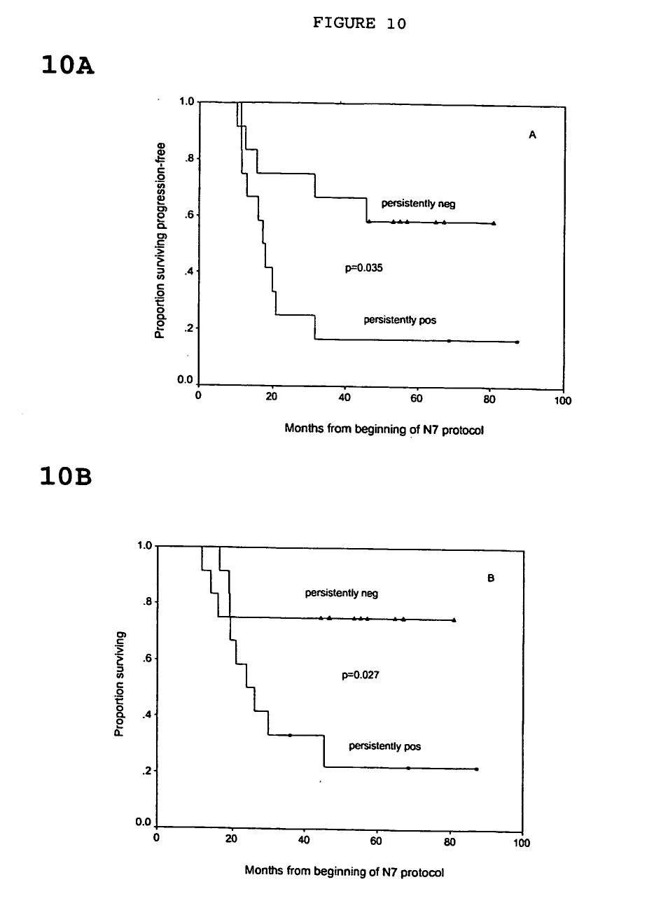

FIG. 10. Kaplan-Meier plots of (10A) PFS and (10B) OS according to GD2 synthase mRNA of stage 4 NB patients (newly diagnosed/without prior relapse) treated with the N7 protocol.

Fourth Series of Experiments

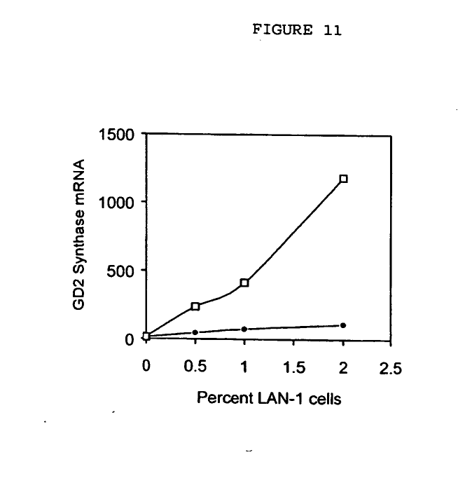

FIG. 11. Quantitation of GD2 synthase mRNA in mononuclear cells seeded with LAN-1 and purged in vitro with 3F8. Samples were incubated for 1 hour at 37° C. in the presence (circle) or absence (square) of 10 ug/ml 3F8.

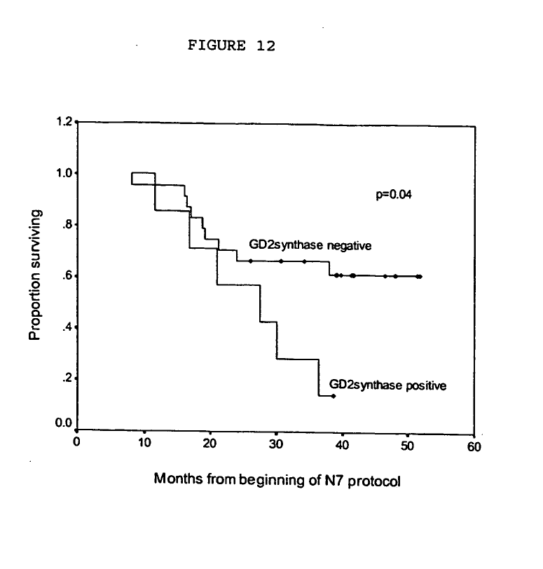

FIG. 12. Kaplan-Meier analysis of overall survival according to GD2 synthase mRNA (pre-purge marrows) from 31 stage 4 NB patients treated with the N7 protocol.

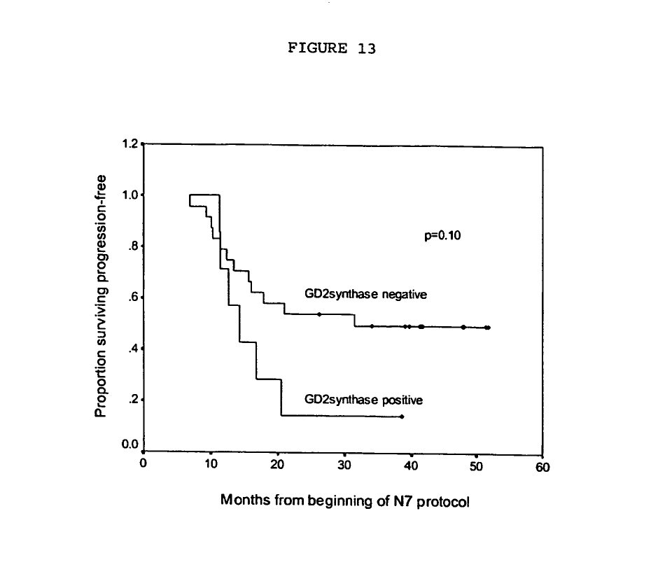

FIG. 13. Kaplan-Meier analysis of progression-free survival according to GD2 synthase mRNA (pre-purge marrows) from 31 stage 4 NB patients treated with the N7 protocol.

Fifth Series of Experiments

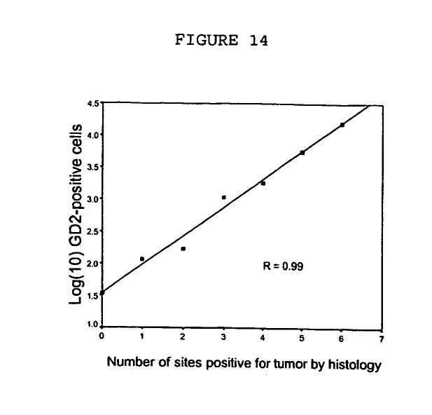

FIG. 14. Correlation of the number of GD2-positive tumor cells (per 10⁶ nucleated marrow cells) and the number of sites (out of 6) positive for tumor by histology.

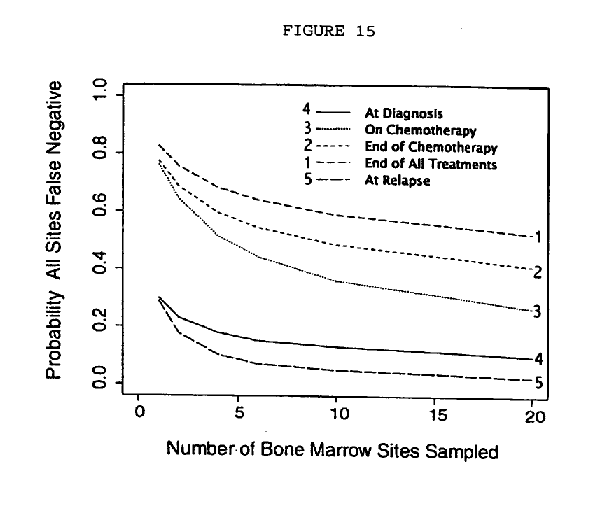

FIG. 15. Probability of falsely declaring the marrow free of tumor versus the number of marrow sites examined by conventional histology.

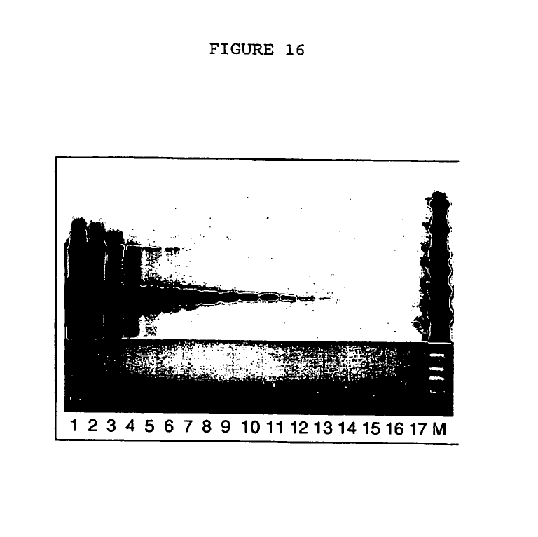

FIG. 16. GAGE1 detection of NB cell line LAN-1-55N in 2-fold serial dilution of RT-PCR product, from 1:2 (lane 1) to 1:2¹⁷ (lane 17). Top: chemiluminescence; Bottom: ethidium bromide staining.

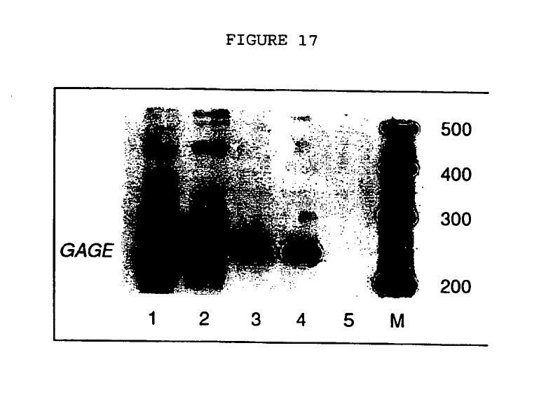

FIG. 17. 10⁶ marrow cells were seeded with 10³ (lane 1), 10² (lane 2), 10 (lane 3), 1 (lane 4), and 0 (lane 5) NB tumor cells. Lane M, molecular weight marker.

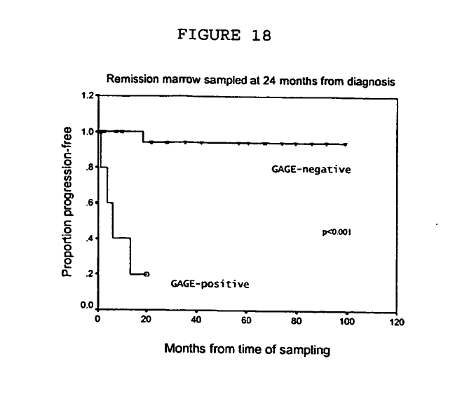

FIG. 18. Relationship between GAGE1 and Kaplan-Meier analysis of progression-free survival from remission marrows sampled at 24 months from diagnosis.

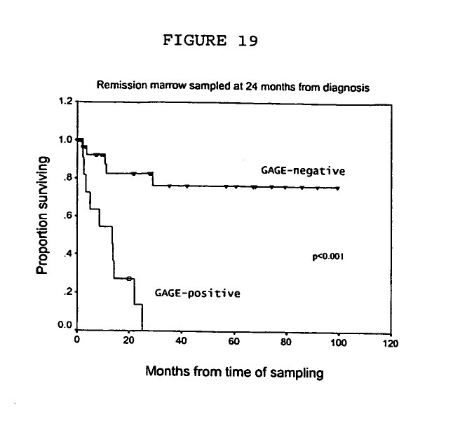

FIG. 19. Relationship between GAGE1 and Kaplan-Meier analysis of survival from remission marrows sampled at 24 months from diagnosis.

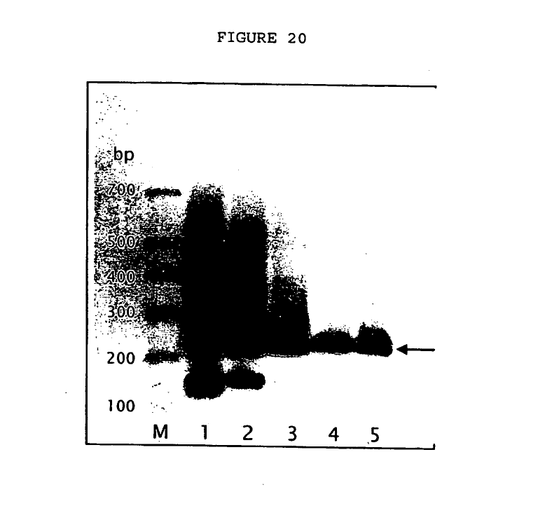

FIG. 20. Sensitivity of GD2 synthase RT-PCR. 10⁶ normal marrow cells were seeded with NB cell line LAN-1 at 10⁴ (lane 1), 10³ (lane 2), 10² (lane 3), 10 (lane 4), and 1 (lane 5). Lane M, molecular weight marker, Arrow, 230 bp.

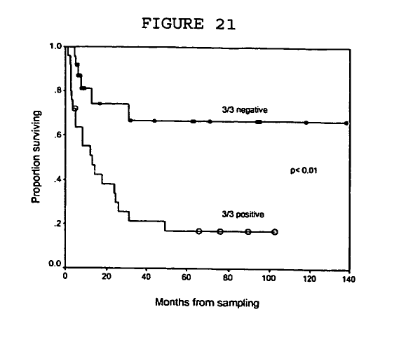

FIG. 21. Kaplan-Meier analysis of survival from the time of sampling based on detection methods HIST, IF, and GD2 synthase RT-PCR, 3/3 tests negative (24 pts, 18 censored); 3/3 tests positive (25 pts, 5 censored); p<0.01.

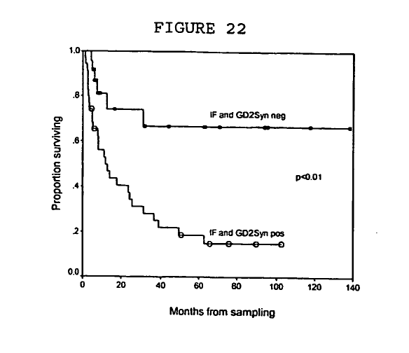

FIG. 22. Kaplan-Meier analysis of survival from the time of sampling based, on detection methods IF and GD2 synthase RT-PCR. 2/2 tests negative (24 pts, 18 censored); 2/2 tests positive (35 pts, 7 censored); p<0.01.

### DETAILED DESCRIPTION OF THE INVENTION

The present invention provides a method to measure GD2 synthase mRNA comprising steps of: (a) obtaining a mRNA sample; (b) performing real-time quantitative RT-PCR on the sample using appropriate primers of GD2 synthase; and (c) determining the amount of GD2 mRNA. The invention further provides a method of the above, where the sample is from a subject, wherein the subject bears a cancer and wherein the cancer is a neuroblastoma.

The invention further provides the method above, wherein, in step (b), the appropriate primers comprise of a pair of sense and antisense primers. The invention also provides the method of the above, wherein the sense primer is 5′ GACAA GCCAG AGCGC GTTA and wherein the antisense primer is 5′-TACTT GAGAC ACGGC CAGGT T-3′.

This invention further yet provides the method above, wherein the real-time quantitative RT-PCR is performed with a probe and wherein the probe was FAM-5′AACCA GCCCT TGCCG AAGGG C-3′.

In addition, this invention provides the above method, wherein the cancer is neuroblastoma, B cell lymphoma, small cell lung cancer, melanoma, osteosarcoma, soft tissue sarcoma, medulloblastoma, high-grade astrocytoma, or retinoblastoma.

Furthermore, this invention provides a method to diagnose a subject which bears cancer expressing GD2 synthase comprising steps of: a) obtaining a mRNA sample from the subject; performing RT-PCR on the sample using appropriate primers of GD2 synthase; and b) determining the amount of GD2 mRNA. In an embodiment, the appropriate primers are sense primer 5′-CCAACTCAACAGGCAACTAC-3′ and antisense primer 5′-GATCATAACGGAGGAAGGTC-3′. In an embodiment, in step (b), the RT-PCR is a real-time quantitative RT-PCR, the appropriate primers comprises a pair of sense and antisense primers, wherein the the sense primer is 5′ GACAA GCCAG AGCGC GTTA and the antisense primer is 5′-TACTT GAGAC ACGGC CAGGT T-3′. In an embodiment, the real-time quantitative RT-PCR is performed with a probe, wherein the probe is FAM-5′AACCA GCCCT TGCCG AAGGG C-3′. In yet another embodiment, the cancer is neuroblastoma, B cell lymphoma, small cell lung cancer, melanoma, osteosarcoma, soft tissue sarcoma, medulloblastoma, high-grade astrocytoma, or retinoblastoma.

This invention provides a method to stage a cancer expressing GD2 synthase in a subject comprising steps of: a) obtaining a mRNA sample from the subject known to carry the disease at different stages; b) performing real-time quantitative RT-PCR on the sample obtained from step (a) using appropriate primers of GD2 synthase; c)determining the amount of the GD2 mRNA; d) comparing the amount obtained in step (c) from the different stages to obtain a standard curve; e) obtaining a mRNA sample from a test subject with the cancer; f) determining the amount of mRNA from the sample using real-time quantitative RT-PCR; and g) comparing the amount determined in step (f) with the standard curve obtained in step (d), thereby determining the stage of the cancer in the test subject.

In an embodiment, the cancer is neuroblastoma, B cell lymphoma, small cell lung cancer, melanoma, osteosarcoma, soft tissue sarcoma, medulloblastoma, high-grade astrocytoma, or retinoblastoma. In another embodiment, the appropriate primers comprises a pair of sense and antisense primers, wherein the sense primer is 5′ GACAA GCCAG AGCGC GTTA, the antisense primer is 5′-TACTT GAGAC ACGGC CAGGT T-3′, the real-time quantitative RT-PCR is performed with a probe, and the probe is FAM-5′AACCA GCCCT TGCCG AAAGGG C-3′.

This invention provides a method for determining tumor cells in a sample comprising steps of: a) obtaining a mRNA sample; b) performing real-time quantitative RT-PCR on the sample using appropriate primers of GD2 synthase; and c) determining the amount of GD2 mRNA, thereby determining the tumor cells in the sample. In an embodiment, in step (b), the appropriate primers comprises a pair of sense and antisense primers, wherein the sense primer is 5′ GACAA GCCAG AGCGC GTTA and the antisense primer is 5′-TACTT GAGAC ACGGC CAGGT T-3′. In another embodiment, the real-time quantitative RT-PCR is performed with a probe, wherein the probe is FAM-5′AACCA GCCCT TGCCG AAGGG C-3′. In an embodiment, the cancer is neuroblastoma, B cell lymphoma, small cell lung cancer, melanoma, osteosarcoma, soft tissue sarcoma, medulloblastoma, high-grade astrocytoma, or retinoblastoma.

This invention further provides a method for determining the minimum residual disease of a cancer comprising steps of: a) obtaining a mRNA sample; b) performing real-time quantitative RT-PCR on the sample using appropriate primers of GD2 synthase; and c) determining the amount of GD2 mRNA, the positive expression of GD2 indicating the residual disease. In an embodiment, in step (b), the appropriate primers comprises a pair of sense and antisense primers, wherein the sense primer is 5′ GACAA GCCAG AGCGC GTTA and the antisense primer is 5′-TACTT GAGAC ACGGC CAGGT T-3′. In another embodiment, the real-time quantitative RT-PCR is performed with a probe, wherein the probe is FAM-5′AACCA GCCCT TGCCG AAGGG C-3′. In yet another embodiment, the cancer is neuroblastoma, B cell lymphoma, small cell lung cancer, melanoma, osteosarcoma, soft tissue sarcoma, medulloblastoma, high-grade astrocytoma, or retinoblastoma.

This invention also provides the above, further comprising determining the level of expression of GAGE1.

This invention provides a kit for detection of GD2 synthase comprising a compartment containing 5′ GACAA GCCAG AGCGC GTTA; and another compartment containing 5′-TACTT GAGAC ACGGC CAGGT T-3′. In an embodiment, the kit further comprises FAM-5′AACCA GCCCT TGCCG AAGGG C-3′.

This invention provides a method for determining the minimum residual disease of a cancer comprising steps of: a) obtaining a mRNA sample; b) performing real-time quantitative RT-PCR on the sample using appropriate primers of a marker; and c) determining the amount of the marker's mRNA, the positive expression of the marker indicating the residual disease. In an embodiment, the marker is WT-1, Midkine, GAGE1 or BAGE.

In another embodiment, in step (b) of the above method, the marker is WT-1. The appropriate primers comprises a pair of sense and antisense primers, wherein the sense primer is 5′-TCC CAG CTT GAA TGC ATG AC-3′ and the antisense primer is 5′-GAT GGG CGT TGT GTG GTT ATC-3′. In another embodiment, the real-time quantitative RT-PCR is performed with a probe, wherein the probe is FAM-CTC GTA CCC TGT GCT GTG GCC CTT T-TAMRA. In yet another embodiment, the cancer is Wilm's tumors, neuroblastoma, Ewing's sarcoma, bladder carcinomas, lung cancers, breast cancers, pancreatic cancers, esophageal cancers, or gastrointestinal cancers.

In another embodiment, the marker is Midkine. The sense primer is 5′-GCT CAG TGC CAG GAG ACC AT-3′ and the antisense primer is 5′-TCC AGG CTT GGC GTC TAG TC-3′. In an additional embodiment, the real-time quantitative RT-PCR is performed with a probe, wherein the probe is FAM-TTC CCT TTC TTG GCT TTG GCC TTT GC-TAMRA.

In yet another embodiment, the marker is GAGE1. The sense primer is 5′-CAA ATG GCG AGA GAC CGT TT-3′ and the antisense primer is 5′-CAT AGG AGC AGC CTG CAA CA-3′. In a separate embodiment, wherein the real-time quantitative RT-PCR is performed with a probe, wherein the probe is FAM-TTG CCC TTC ACA TGC CAC AGA TGA TAG G-TAMRA. In another embodiment, the cancer is melanoma, non-small cell lung cancer, sarcoma, bladder cancers, head and neck tumors, or neuroblastoma.

In a further embodiment, the marker is BAGE. The sense primer is 5′-GAT GGT GGT GGC AAC AGA GA-3′ and the antisense primer is 5′-TTC ATC AGC CTG GCT TGG A-3′. In still another embodiment, the real-time quantitative RT-PCR is performed with a probe, wherein the probe is FAM-AGA AAA ACC GCT CTG GCC GCC AT-TAMRA. In a separate embodiment, the cancer is melanoma, non-small cell lung cancer, sarcoma, bladder cancers, head and neck tumors, or neuroblastoma.

This invention also provides a method for determining tumor cells in a sample comprising steps of: a) obtaining a mRNA sample; b) performing real-time quantitative RT-PCR on the sample using appropriate primers of a marker; and c) determining the amount of markers mRNA, thereby determining the tumor cells in the sample. In an embodiment, the marker is WT-1, Midkine, GAGE1 or BAGE.

In another embodiment, in step (b) of the above method, the marker is WT-1. The appropriate primers comprises a pair of sense and antisense primers. In yet another embodiment, the sense primer is 5′-TCC CAG CTT GAA TGC ATG AC-3′ and the antisense primer is 5′-GAT GGG CGT TGT GTG GTT ATC-3′. In a separate embodiment, the real-time quantitative RT-PCR is performed with a probe, wherein the probe is FAM-CTC GTA CCC TGT GCT GTG GCC CTT T-TAMRA.

In yet another embodiment, the cancer is Wilm's tumors, neuroblastoma, Ewing's sarcoma, bladder carcinomas, lung cancers, breast cancers, pancreatic cancers, esophageal cancers, or gastrointestinal cancers.

In another embodiment, the marker is Midkine. The sense primer is 5′-GCT CAG TGC CAG GAG ACC AT-3′ and the antisense primer is 5′-TCC AGG CTT GGC GTC TAG TC-3′. In yet another embodiment, the real-time quantitative RT-PCR is performed with a probe, wherein the probe is FAM-TTC CCT TTC TTG GCT TTG GCC TTT GC-TAMRA.

In another embodiment, the marker is GAGE1. The sense primer is 5′-CAA ATG GOG AGA GAC CGT TT-3′ and the antisense primer is 5′-CAT AGG AGC AGC CTG CAA CA-3′. In a further embodiment, the real-time quantitative RT-PCR is performed with a probe, wherein the probe is FAM-TTG CCC TTC ACA TGC CAC AGA TGA TAG G-TAMRA. In another embodiment, the cancer is melanoma, non-small cell lung cancer, sarcoma, bladder cancers, head and neck tumors, or neuroblastoma.

In a further embodiment, the marker is BAGE. The sense primer is 5′-GAT GGT GGT GGC AAC AGA GA-3′ and the antisense primer is 5′-TTC ATC AGC CTG GCT TGG A-3′. In another embodiment, the real-time quantitative RT-PCR is performed with a probe and the probe is FAM-AGA AAA ACC GCT CTG GCC GCC AT-TAMRA. In an embodiment, the cancer is melanoma, non-small cell lung cancer, sarcoma, bladder cancers, head and neck tumors, or neuroblastoma.

This invention also provides a kit for detection of WT-1 comprising a compartment containing 5′-TCC CAG CTT GAA TGC ATG AC-3′; and another compartment containing 5′-GAT GGG CGT TGT GTG GTT ATC-3′. In an embodiment, the kit further comprises FAM-CTC GTA CCC TGT GCT GTG GCC CTT T-TAMRA.

This invention further provides a kit for detection of Midkine comprising a compartment containing 5′-GCT CAG TGC CAG GAG ACC AT-3′; and another compartment containing 5′-TCC AGG CTT GGC GTC TAG TC-3′. In an embodiment, the kit further comprises FAM-TTC CCT TTC TTG GCT TTG GCC TTT GC-TAMRA.

This invention provides a kit for detection of GAGE1 comprising a compartment containing 5′-CAA ATG GCG AGA GAC CGT TT-3′; and another compartment containing 5′-CAT AGG AGC AGC CTG CA CA-3′. In an embodiment, the kit further comprising FAM-TTG CCC TTC ACA TGC CAC AGA TGA TAG G-TAMRA.

This invention also provides a kit for detection of BAGE comprising a compartment containing 5′-GAT GGT GGT GGC AAC AGA GA-3′; and another compartment containing S′-TTC ATC AGC CTG GCT TGG A-3′. In an embodiment, the kit further comprising FAM-AGA AAA ACC GCT CTG GCC GCC AT-TAMRA.

This invention also provides a method to measure mRNA of WT-1, Midkine, GAGE1, or BAGE comprising steps of: a obtaining an mRNA sample; b. performing real-time quantitative RT-PCR on the sample using appropriate primers of WT-1, Midkine, GAGE1, or BAGE; and c. determining the amount of WT-1, Midkine, GAGE1, or BAGE.

Finally, this invention provides a method to diagnose a subject which bears cancer expressing WT-1, Midkine, GAGE1, or BAGE, comprising steps of: a. obtaining a mRNA sample from the subject; performing RT-PCR on the sample using appropriate primers of WT-1, Midkine, GAGE1, or BAGE; and b. determining the amount of mRNA of WT-1, Midkine, GAGE1, or BAGE.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

### EXPERIMENTAL DETAILS AND METHODS

First Series Of Experiments

Quantitation of Marrow Disease in Neuroblastoma by Real-time RT-PCR

Purpose:

GD2 is abundantly expressed in neuroblastoma (NB). GD2 synthesis is dependent on a key enzyme β1,4-N-acetylgalactosaminyltransferase (GD2 synthase). The potential of GD2 synthase mRNA as a molecular marker of minimal residual disease (MRD) is explored by first comparing it quantitatively with immunocytology, and by testing its clinical utility.

Experimental Design:

A real-time RT-PCR assay to quantify mRNA of GD2 synthase was developed. Quantitation was normalized to endogenous control glyceraldehyde-3-phosphate dehydrogenase in a multiplex PCR.

Results:

The upper limit of normal was defined by 31 normal marrow and blood samples, achieving a sensitivity of one NB cell in 10⁶ normal mononuclear cells. When 155 bone marrows from 100 NB patients were studied, GD2 synthase mRNA levels correlated well with the number of GD2-positive cells, as measured by immunocytology using anti-GD2 antibodies (r=0.96). This is the first demonstration on the quantitative relationship between a specific mRNA and the actual number of tumor cells. In a pilot study, the level of this transcript in sequential marrow samples of five stage 4 NB patients correlated closely with their clinical status. At 24 months from diagnosis, available remission bone marrows from patients with advanced NB diagnosed at >1 year of age initially treated with protocols N6 and N7 at Memorial Sloan-Kettering Cancer Center (n=44) were analyzed for GD2 synthase mRNA. Positivity was strongly associated with progression-free (p<0.005) and overall survival (p<0.001).

Conclusions:

Measurement of tumor cells by real-time quantitative RT-PCR of GD2 synthase has potential clinical utility, especially for the detection of MRD.

Patients and Methods

Patients.

Neuroblastoma patients evaluated at Memorial Sloan-Kettering Cancer Center were diagnosed and staged in accordance with the International Neuroblastoma Staging System (25). Serial bone marrows were obtained as part of disease evaluation while the patient was being treated, with approval by the institutional review board of Memorial Hospital. Each marrow examination generally consisted of six samplings (two biopsies: right and left posterior iliac crest; and four aspirates: right and left anterior iliac crest, right and left posterior iliac crest obtained from six different sites of the iliac crests). Details of the procedure were described previously (9).

Immunocytology.

Freshly collected heparinized bone marrow pooled from four aspiration sites was separated by ficoll centrifugation. Mononucleated cells were incubated with a panel of anti-GD2 monoclonal antibodies, followed by a reaction with a fluoresceinated anti-mouse IgG+IgM antibody. GD2-positive tumor cells were examined and enumerated using a fluourescence microscope by a trained technician. Quantitation was expressed in GD2 positive cells divided by the total number of mononuclear cells in each counting chamber. Negative detection was defined as <0.001% (9).

mRNA Extraction and cDNA Synthesis.

Cryopreserved bone marrows mononuclear cells were used. Total RNAs were extracted and reverse transcription performed as previously described (26).

Real-time Quantitative PCR

I. Background.

Relative quantitation of GD2 synthase mRNA was achieved by means of the ABI Prism 7700 Sequence Detection System (Applied Biosystems, Foster City, Calif.). In TaqMan real-time quantitation technology (27-29), the 5′exonuclease activity of the Taq polymerase cleaves and releases the hybridization probe that was labeled with a fluorescent reporter dye. This fluorogenic probe is specific for the target sequence, thereby generating a fluorescence signal that is specific and is directly proportional to the amount of PCR product synthesized. PCR reactions are characterized by the time-point during cycling when amplification of the PCR product is first detected, rather than the amount of product accumulated after a fixed number of cycles. Since the amount of product at the exponential phase of the PCR is proportional to the initial copy number of the target, the more abundant the starting quantity of a target, the earlier will the PCR amplification be detected by means of the fluorescence signal. In this technology, the target quantity is measured by identifying the threshold cycle number (C_{T}), i.e. when the fluorescence signal crosses a preset detection threshold. The laser detector of the Prism 7700 monitors the cycle to cycle change in fluorescence signal on-line. The fewer cycles it takes to reach a detectable level of fluorescence, the greater the initial copy number.

Measurement of GD2 synthase transcript was based on two reporter dyes with the largest difference in emission wavelength maxima, namely 6-FAM for GD2 synthase and VIC for glyceraldehyde-3-phosphate dehydrogenase (GAPDH), our endogenous reference to control for difference in mRNA extraction and cDNA synthesis. After optimization by limiting the primer concentrations of the more abundant target GAPDH, multiplex PCR became the standard assay, resulting in higher throughput and reducing the effect of pipetting errors.

The primers and probe for GD2 synthase were designed using the applications-based primer design software Primer Express (Applied Biosystems, ABI). The probe spanned an intron, thereby avoiding the amplification of contaminating genomic DNA present in the sample. GD2 synthase sense primer was 5′-GACAAGCCAGAGCGCGTTA-3′, antisense primer was 5′-TACTTGAGACACGGCCAGGTT-3′. Probe was FAM-5′AACCAGCCCTTGCCGAAGGGC-3′ (99 bp). GAPDH sense primer was 5′-GAAGGTGAAGGTCGGAGTC-3′, and antisense was 5′-GAAGATGGTGATGGGATTTC-3′. Probe was VIC-5′CAAGCTTCCCGTTCTCAGCC-3′ (226 bp). GD2 synthase and GAPDH designs were based sequence from GENBank, accession NM_{—}001478 and J04038, respectively. Primers and probes were synthesized by ABI.

II. Procedure.

In each 25 ul MicroAmp optical tubes (ABI), 2 ul cDNA template was added to a PCR mix. This mixture included the Taqman master mix (ABI) containing 5 mM MgCl₂, 200 uM each dATP, dCTP, dGTP, and dUTP, 0.05 U/ul AmpliTaq Gold DNA polymerase, as well as 0.01 U/ul AmpErase uracil-N-glycosylase (UNG) to prevent PCR product carryover, as well as a passive reference dye ROX. This reference dye provides an internal reference to which the reporter dye signal can be normalized, compensating for the fluorescence between wells and between experiments caused by pipetting errors or instrument variability. Also included in the mix was 300 nM each GD2 synthase forward and reverse primers, 200 nM GD2 synthase (FAM) probe, and 40 nM each GAPDH primers and 100 nM GAPDH (VIC) probe. Each tube was covered with a MicroAmp optical cap. Every PCR run included a 5-point standard to generate a standard curve for GD2 synthase and for GAPDH, plus a no template control. Samples were often run in duplicate PCR experiments.

Using the ABI Prism 7700 Sequence Detector, the initial PCR began with a 50° C., 2 minutes step to optimize UNG activity, followed by a 95° C., 10 minutes step to activate AmpliTaq Gold DNA polymerase and UNG deactivation. Then 40 cycles at 95° C. for 15 seconds and 60° C. for 1 minute followed. The entire PCR took 2 hours to complete with no post-PCR handling.

Calculation.

For each unknown test sample, the amount of GD2 synthase and endogenous reference GAPDH was determined from the respective standard curve. Dividing the GD2 synthase level by the GAPDH level resulted in a normalized GD2 synthase value. Quantitation of 31 normal bone marrow and peripheral blood established the threshold below which the quantitative value was considered background. The variation in the quantitation from experiment to experiment was within 15%.

Statistical Analysis.

Prognostic importance of clinical variables was evaluated by Cox regression using univariate and multivariate analyses. Patient survival was estimated by the Kaplan-Meier method and survival comparisons between groups by the log-rank test.

### Results

Specificity and Sensitivity of Real-time Quantitative RT-PCR of GD2 Synthase Transcript.

A multiplex assay was established by generating two reproducible standard curves, one for GD2 synthase mRNA and one for GAPDH. The cDNA standard (100,000 arbitrary units) was derived from a neuroblastoma cell line NMB7, and was reverse transcribed in the same manner as the test samples. The standard was serially diluted to obtain a linear dynamic range of >5 logs (FIG. 1). The amplification plots of cDNA standards of GD2 synthase and GAPDH are illustrated in FIGS. 2A and 2B. The GD2 synthase transcript level was expressed as a multiple of GAPDH expression. The threshold level, i.e. the upper limit of normal (mean+2 standard error (SE)) of GD2 synthase mRNA was established using a total of 31 normal BM and peripheral blood samples. Mean GD2 synthase/GAPDH was 0.98 and SE was 1.60. Thus, the calculated threshold of the normalized GD2 synthase was 4.18 (rounded to 5.0 for this analysis). Levels below 5.0 were defined as negative. Sensitivity of this assay was established by spiking NMB7 cells at ratios ranging from 1 to 10,000 tumor cells per million normal marrow mononuclear cells. The quantitative values are tabulated in Table 1. The level of GD2 synthase transcript for a tumor content of 1 in 10⁶ was 9.54±1.74.

TABLE 1
Sensitivity of real-time quantitative
RT-PCR of GD2 synthase mRNA

| Ratio of tumor cells to | |
|---|---|
| normal marrow cells | GD2 synthase/GAPDH |
| 10⁻² | 581.91 ± 62.41^{a} |
| 10⁻³ | 93.24 ± 4.75 |
| 10⁻⁴ | 24.18 ± 2.06 |
| 10⁻⁵ | 12.18 ± 1.51 |
| 10⁻⁶ | 9.54 ± 1.74 |

^{a}mean ± SE of 2 experiments

Quantitation of GD2 Synthase mRNA in 155 BM from 100 NB Patients.

The level of GD2 synthase mRNA was correlated with the number of GD2-positive tumor cells as enumerated by immunocytology. In this real-time quantitative RT-PCR, levels above 5.0 were defined as positive, while measurements above 0.001% GD2-positive cells by immunocytology were deemed positive. Only marrows positive for both GD2 synthase and immunocytology were included in this correlation analysis. As shown in FIG. 3, a linear relationship was found between these two independent measurements (r=0.96).

There was concordance in both positivity and negativity between GD2 synthase mRNA by real-time quantitative RT-PCR and immunocytology. Agreement was 74% (115/155 BM) (Table 2). Thirty-two samples were GD2 synthase positive and immunocytology negative; 8 samples were GD2 synthase negative and immunocytology positive. Interestingly, the BM aspirate examined in 7 of the above 8 samples were also negative by histological examination.

TABLE 2
Concordance in molecular versus immunologic
detection of GD2 in the bone marrow samples

| | Immunocytology | | |
|---|---|---|---|
| Real-time RT-PCR | Positive | Negative | Total |
| Positive | 45 | 32 | 77 |
| Negative | 8 | 70 | 78 |
| Total | 53 | 102 | 155 |

Patterns of GD2 Synthase mRNA Measurement of Patients with Serial Bone Marrow Samplings.

In a pilot study, sequential bone marrows from five stage 4 NB patients were studied throughout the course of their treatment and follow-up (Table 3). The expression levels of GD2 synthase correlated closely with the patient's clinical status. Patient 1, 2 and 3 are alive and progression-free. Patient 4 and 5 have succumbed to NB and died.

TABLE 3
Relative quantitation of GD2 synthase mRNA of sequential samples in
the bone marrows of stage 4 neuroblastoma patients during the course
of treatment and follow-up^{a}

Patient

| Timeline | #1 | #2 | #3 | #4 | #5 |
|---|---|---|---|---|---|
| At diagnosis | 24.7 (0 m)^{b} | 21.0 (0 m) | 107.1 (0 m) | 4098.3 (0 m) | 350.3 (0 m) |
| During treatment | 12.0 (6 m) | 0 (6 m) | 9.9 (6 m) | 37.4 (6 m) | 15.0 (1 m) |
| Clinical remission | 1.8 (12 m) | 0 (12 m) | 0 (12 m) | 0 (13 m) | 2.2 (10 m) |
| Follow-up | 4.2 (17 m) | 0 (18 m) | 0 (18 m) | 50.8 (17 m) | 8.5 (13 m) |
| Follow-up | 1.1 (24 m) | 0 (31 m) | 0 (24 m) | 16.2 (24 m) | — |
| Relapse | — | — | — | 25.8 (30 m) | 546.3 (18 m) |
| Relapse | — | — | — | 26.1 (41 m) | — |
| Status | PF^{c} | PF | PF | Dead | Dead |
| | (73 m) | (83 m) | (100 m) | (50 m) | (22 m) |

^{a}Relative quantitation was expressed as GD2 synthase/GAPDH with a detection threshold of 5.0, as described in the Patients and Methods section.
^{b}Months (m) from diagnosis
^{c}Progression-free

Prognostic Significance of GD2 Synthase Transcript on the Survival of Patients with NB.

At 24 months from diagnosis, available remission bone marrows from patients with advanced NB diagnosed at >1 year of age initially treated with protocol N6 (n=19) and protocol N7 (n=25) were analyzed for GD2 synthase mRNA by real-time quantitative RT-PCR. Eight of nineteen (42%) N6 patients and five of twenty-five (20%) N7 patients were positive, as defined by the threshold of 5.0. In total, 30% of the samples (13/44) was positive for GD2 synthase mRNA. FIGS. 4 and 5 showed strong correlation between GD2 synthase positivity at 24 months from diagnosis and adverse clinical outcome, for both progression-free survival (p<0.005) and overall survival (p<0.001).

### Discussion

As the focus of curative strategies shifts to minimal residual disease (MRD), objective quantitation of occult tumor cells becomes critically important. For neuroblastoma, the efficacy of various therapeutic approaches is often based on bone marrow response. In particular, autologous marrow/stem cell transplantation is predicated on the marrow being free of tumor cells (30). As in other metastatic cancers, BM is often the first site of relapse, and not uncommonly the only site of metastasis for NB. Large retrospective studies have clearly demonstrated the importance of marrow remission in ensuring long-term survival (31). As therapy becomes more effective, being able to quantify tumor cells in sequential follow-up BM will help define the quality of remission by serving as a surrogate marker for tumor response. Patients in solid remission may be spared further chemotherapy and its leukemia risk. Sensitive methods to detect NB cells can further provide better timing for marrow collection prior to autologous stem cell transplant. At time of minimal residual disease, absence of detectable molecular marker may serve as a surrogate endpoint for adjuvant treatment strategy. Moreover, quantitative information on BM disease will help detect relapse earlier to improve the patient's chance of survival.

Immunocytology using specific antibodies against GD2 has been successful in detecting and quantifying tumor cells in the BM (9, 10). However, only freshly collected samples can be used. This technique is also labor intensive because it requires counting cells under the microscope. In contrast, molecular monitoring of residual tumor cells by RT-PCR utilizes cryopreserved mononuclear cells, and experiments can be repeated multiple times and for multiple markers as they are being developed. Sensitivity of tumor cell detection by immunocytology is 1/10⁵, whereas as RT-PCR is 1/10⁶. However, molecular based MRD assays can be hampered by false positive results, due to the fact that tumor-specific markers that detect rare tumor cells can also be present in non-tumor cells. In addition, there is the process of illegitimate transcription, i.e. the transcription of any gene in any cell type. Tissue-specific markers can also lead to false positive PCR results, if normal cells are introduced in the circulation after invasive procedures.

In our laboratory, cancer-testis antigens MAGE (13), GAGE1 (26), SSX (32), BAGE, and NYESO (data not shown) have been examined to evaluate their potentials as markers for MRD. Among these antigens, GAGE1 was found to be a superior molecular marker because its presence in blood and BM had prognostic importance in disease progression and survival for patients with advanced NB (33) and melanoma (34). GAGE1 detection was based on RT-PCR and chemiluminescence, where positivity was identified by PCR product of the appropriate size, and confirmed by Southern blotting.

Development of a real-time quantitative RT-PCR assay broadens the potential in the monitoring of MRD in NB. The advantages of real-time quantitation are numerous. With a wide linear dynamic range, superior sensitivity and accuracy, it allows good intra-assay and inter-assay reproducibility. Additional attractions include high throughput capacity, speed, and elimination of lengthy post-PCR handling steps, preventing potential carryover contamination. In this study, a new molecular marker, GD2 synthase, was explored to measure neuroblastoma cells in the bone marrow by real-time quantitative RT-PCR. Over the last 15 years, our laboratory has routinely used anti-GD2 immunocytology to enumerate NB cells in the BM of patients. While anti-GD2 monoclonal antibodies are specific for the oligosaccharide moiety of the antigen GD2, real-time quantitation of GD2 synthase mRNA provides us information on the expression of the enzyme. The excellent quantitative correlation between these two measurements suggests a close relationship between the enzyme GD2 synthase and the antigen GD2 in individual tumor cells and among patients. Thus, comparison over time in the same patient can be made, as well as comparison through different phases of treatment, and among patients. This report is the first demonstration of a quantitative relationship between a specific mRNA and the actual number of tumor cells. Because of the inherent sensitivity of RT-PCR, it was not surprising to find more samples that were GD2 synthase positive and immunocytology negative. These were unlikely to be false positive samples since these patients had other evidence of disease even though some of their marrows were histologically negative.

Clinical utility of this method was demonstrated by first examining sequential BM samples of individual NB patients. The level of transcript agreed with their clinical disease status, and it correlated closely with the tumor burden in the marrow, as measured by immunocytology (data not shown). Secondly, the importance of remission marrow GD2 synthase mRNA among patients two years from diagnosis was tested. This cohort had previously been monitored for GAGE1 expression by RT-PCR and chemiluminescence. GAGE1 positivity of their marrows at 24 month after diagnosis was strongly correlated with disease progression and death (33). GD2 synthase expression was also significantly associated with patient outcome. Interestingly, there were a statistically lower percentage of GD2 synthase positive marrows among patients entered in protocol N7 compared to those in N6. Since these two protocols were essentially identical, except for the inclusion of I-131-labeled monoclonal antibody 3F8 in N7, this difference may be a reflection of the treatment efficacy of the two protocols.

This newly developed quantitative RT-PCR assay has many potential clinical utilities. By quantifying tumor cells in the marrow during the course of treatment and follow-up, substantially more information on disease status will be gained. Because of the intensified use of topoisomerase-II inhibitors and alkylators, there is an increase in the incidence of secondary leukemia among NB patients (35). Patients early on in solid remission may not need further chemotherapy. Adjuvant therapies, such as monoclonal antibody, oral VP16, or cis-retinoic acid, often make gradual, and not quantum changes in MRD, which by definition is beyond the sensitivity of conventional histologic or radiographic techniques. The efficacy and duration of adjuvant treatment, which can only be assessed retrospectively after an extended period of clinical follow-up, may now be determined by using the level of GD2 synthase transcript as the surrogate endpoint. As to the patients who may be at risk for relapse, albeit systemic or central nervous system, they may benefit from earlier intervention/prophylaxis, if an elevation in the molecular marker in bone marrow, blood, or CSF is noted. Moreover, better timing for marrow or stem cell collection may be possible. Because GD2 is present in other malignancies, including osteosarcoma (36), soft tissue sarcoma (37), medulloblastoma and high-grade astrocytoma (38, 39), retinoblastoma (40), melanoma (41, 42), and small cell lung cancer (43), this quantitative assay may have broader clinical applications.

### REFERENCES

1. Diel, I. J., Kaufmann, M., Costa, S. D., Holle, R., von Minckwitz, G., Solomayer, E. F., Kaul, S., Bastert, G. Micrometastatic breast cancer cells in bone marrow at primary surgery: prognostic value in comparison with nodal status. J. Natl. Cancer Inst., 88:1652-8, 1996.

2. Lindemann, F., Schlimok, G., Dirschedl, P., Witte, J., Riethmueller, G. Prognostic significance of micrometastatic tumour cells in bone marrow of colorectal cancer patients. Lancet, 340:685-689, 1992.

3. Jauch, K. W., Heiss, M. M., Gruetzner, U., Funke, I., Pantel, K., Babic, R., Eissner, H. J., Giethmueller, G., Schildberg, F. W. Prognostic significance of bone marrow micrometastases in patients with gastric cancer. J. Clin. Oncol., 14:1810-1817, 1996.

4. Pantel, K., Izbicki, J., Passlick, B., Angstwurm, M., Haussinger, K., Thetter, O., Riethmueller, G. Frequency and prognostic significance of isolated tumour cells in bone marrow of patients with non-small cell lung cancer without overt metastases. Lancet, 347:649-653, 1996.

5. Ghossein, R., Coit, D., Brennan, M., Zhang, Z. F., Wang, Y., Bhattacharya, S., Houghton, A., Rosai, J. Prognostic significance of peripheral blood and bone marrow tyrosinase messenger RNA in malignant melanoma. Clin. Cancer Res., 4:419-428, 1998.

6. Gruhn, B., Hongeng, S., Yi, H., Hancock, M. L., Rubnitz, J. E., Neale, G. A., Kitchingman, G. R. Minimal residual disease after intensive induction therapy in childhood acute lymphoblastic leukemia predicts outcome. Leukemia, 12:675-81, 1998.

7. Hartman, O., Valteau-Couanet, D., Vassal, G., Lapierre, V., Brugieres, L., Delgado, R., Couanet, D., Lumbroso, J., Benhamou, E. Prognostic factors in metastatic neuroblastoma in patients over 1 year of age treated with high-dose chemotherapy and stem cell transplantation: a multivariate analysis in 218 patients treated in a single institution. Bone Marrow Transplant., 23:789-795, 1999.

8. Cotterill, S. J., Pearson, A. D. J., Pritchard, J., Foot, A. B. M., Roald, B., Kohler, J. A., Imeson, J. Clinical prognostic factors in 1277 patients with neuroblastoma: results of the European Neuroblastoma Study Group “Survey” 1982-1992. Eur. J. Cancer, 36:901-908, 2000.

9. Cheung, N. K., Heller, G., Kushner, B. H., Liu, C., Cheung, I. Y. Detection of metastatic neuroblastoma in bone marrow: when is routine marrow histology insensitive? J. is Clin. Oncol., 15:2807-2817, 1997.

10. Faulkner, L. B., Tintori, V., Tamburini, A., Paoli, A., Garaventa, A., Viscardi, E., Tucci, F., Lippi, A. A., De Bernardi, B., Bernini, G. High-sensitivity immunocytologic analysis of neuroblastoma cells in paired blood and marrow samples. J. Hematother., 7:361-366, 1998.

11. Miyajima, Y., Kato, K., Numata, S. I., Kudo, K., Horibe, K. Detection of neuroblastoma cells in bone marrow and peripheral blood at diagnosis by the reverse transcriptase-polymerase chain reaction for tyrosine hydroxylase mRNA. Cancer, 75:2757-2761, 1995.

12. Lode, H. N., Bruchelt, G., Seitz, G., Gebhardt, S., Gekeler, V., Niethammer, D., Beck, J. Reverse transcriptase-polymerase chain reaction analysis of monoamine transporters in neuroblastoma cell lines: correlations to meta-iodobenzylguanidine uptake and tyrosine hydroxylase gene expression. Eur. J. Cancer, 31A:586-590, 1995.

13. Cheung, I. Y., Barber, D., Cheung, N. K. V. Detection of microscopic neuroblastoma in marrow by histology, immunocytology, and RT-PCR of multiple molecular markers. Clin. Cancer Res., 4:2801-2805, 1998.

14. Ladetto, M., Donovan, J. W., Harig, S., Trojan, A., Poor, C., Schlossnan, R., Anderson, K. C., Gribben, J. G. Real-time polymerase chain reaction of immunoglobulin rearrangements for quantitative evaluation of minimal residual disease in multiple myeloma. Biol. Blood Marrow Transplant., 6:241-53, 2000.

15. Verhagen, O. J., Willemse, M. J., Breunis, W. B., Wijkhuijs, A. J., Jacobs, D. C., Joosten, S. A., van Wering, E. R., van Dongen, J. J., van der Schoot, C. E. Application of germline IGH probes in real-time quantitative PCR for the detection of minimal residual disease in acute lymphoblastic leukemia. Leukemia, 14:1426-1435, 2000.

16. Emig, M., Saussele, S., Wittor, H., Weisser, A., Reiter, A., Willer, A., Berger, U., Hehlmann, R., Cross, N. C., Hochhaus, A. Accurate and rapid analysis of residual disease in patients with CML using specific fluorescent hybridization probes for real time quantitative RT-PCR. Leukemia, 13:1825-32, 1999.

17. Lo, Y. M. D., Chan, L. Y. S., Chan, A. T. C., Leung, S, F, Lo, K. W., Zhang, J., Lee, J. C. K., Hjelm, M., Johnson, P. J., Huang, D. P. Quantitative and temporal correlation between circulating cell-free Epstein-Barr virus DNA and tumor recurrence in nasopharyngeal carcinoma. Cancer Res., 59:5452-5455, 1999.

18. Wattjes, M. P., Krauter, J., Nagel, S., Heidenreich, O., Ganser, A., Heil, G. Comparison of nested competitive RT-PCR and real-time RT-PCR for the detection and qualification of AML1/MTG8 fusion transcripts in t(8;21) positive acute myelogenous leukemia. Leukemia, 14:329-335, 2000.

19. Wu, Z., Schwartz, E., Seeger, R. C., Ladisch, S. Expression of GD2 ganglioside by untreated primary human neuroblastomas. Cancer Res., 46:440-443, 1986.

20. Kramer, K., Gerald, W., Kushner, B. H., Larson, S. M., Hameed, M., Cheung, N. K. V. Disialoganglioside GD2 loss following monoclonal antibody therapy is rare in neuroblastoma. Clin. Cancer Res., 4:2135-2139, 1998.

21. Furukawa, K., Soejima, H., Niikawa, N., Shiku, H., Furukawa, K. Genomic organization and chromosomal assignment of the human β1,4-N-acetylgalactosaminyltransferase gene. J. Biol. Chem., 271:20836-20844, 1996.

22. Yamashiro, S., Ruan, S., Furukawa, K., Tai, T., Lloyd, K. O., Shiku, H., Furukawa, K. Genetic and enzymatic basis for differential expression of GM2 and GD2 gangliosides in human cancer cell lines. Cancer Res., 53:5395-5400, 1993.

23. Yuyama, Y., Dohi, T., Morita, H., Furukawa, K., Oshima, M. Enhanced expression of GM2/GD2 synthase mRNA in human gastrointestinal cancer. Cancer, 75:1273-1280, 1995.

24. Kuo, C. T., Bostick, P. J., Irie, R. F., Morton, D. L., Conrad, A. J., Hoon, D. S. B. Assessment of messenger RNA of β1,4-N-acetylgalactosaminyltransferase as a molecular marker for metastatic melanoma. Clin. Cancer Res., 4:411-418, 1998.

25. Brodeur, G., Pritchard, J., Berthold, F., Carlen, N. L. T., Castel, V., Castleberry, R. P., DeBernardi, B., Evans, A. E., Favrot, M., Hedborg, F., Kaneko, M., Kemshead, J., Lampert, F., Lee, R. E. J., Look, T., Pearson, A. D. J., Philip, T., Roald, B., Sawada, T., Seeger, R. C., Tsuchida, Y., Voute, P. A. Revisions of the international criteria for neuroblastoma diagnosis, staging and response to treatment. J. Clin. Oncol., 11:1466-1477, 1993.

26. Cheung, I. Y., Cheung, N. K. V. Molecular detection of GAGE1 expression in peripheral blood and bone marrow: utility as a tumor marker for neuroblastoma. Clin. Cancer Res., 3:821-826, 1997.

27. Heid, C. A., Stevens, J., Livak, K. J., Williams, P. M. Real time quantitative PCR. Genome Res., 6:986-94, 1996.

28. Gibson, U. E., Heid, C. A., Williams, P. M. A novel method for real time quantitative RT-PCR. Genome Res., 6:995-1001, 1996.

29. Holland, P. M., Abramson, R. D., Watson, R., Gelfand, D. H. Detection of specific polymerase chain reaction product by utilizing the 5′-3′ exonuclease activity of Thermus aquaticus DNA polymerase. Proc. Natl. Acad. Sci. USA, 88:7276-7280, 1991.

30. Faulkner, L. B., Garaventa, A., Paoli, A., Tintori, V., Tamburini, A., Lacitignola, L., Veltroni, M., Lo Piccolo, M. S., Viscardi, E., Milanaccio, C., Tondo, A., Spinelli, S., Bernini, G., De Bernardi, B. In vivo cytoreduction studies and cell sorting-enhanced tumor-cell detection in high-risk neuroblastoma patients: implications for leukapheresis strategies. J. Clin. Oncol., 18:3829-3836, 2000.

31. Ladenstein, R., Philip, T., Lasset, C., Hartmann, O., Garaventa, A., Pinkerton, R., Michon, J., Pritchard, J., Klingebiel, T., Kremens, B., Pearson, A., Coze, C., Paolucci, P., Frappaz, D., Gadner, H., Chauvin, F. Multivariate analysis of risk factors in stage 4 neuroblastoma patients over the age of one year treated with megatherapy and stem-cell transplantation: A report from the European Bone Marrow Transplantation Solid Tumor Registry. J. Clin. Oncol., 16:953-965, 1998.

32. Chi, S. N., Cheung, I. Y., Cheung, N. K. V. SSX-2 and SSX-4: cancer-testis antigens as tumor markers of neuroblastoma. J. Pediatr. Hematol. Oncol., 22:380, 2000.

33. Cheung, I. Y., Chi, S. N., Cheung, N. K. V. Prognostic significance of GAGE1 detection in bone marrows on survival of patients with metastatic neuroblastoma. Med. Ped. Oncol., 35:632-634, 2000.

34. Cheung, I. Y., Cheung, N. K. V., Ghossein, R. A., Satagopan, J. M., Bhattacharya, S., Coit, D. G. Association between molecular detection of GAGE1 and survival in patients with malignant melanoma: A retrospective cohort study. Clin. Cancer Res., 5:2042-2047, 1999.

35. Kushner, B. H., Cheung, N. K. V., Kramer, K., Heller, G., Jhanwar, S. C. Neuroblastoma and treatment-related myelodysplasia/leukemia: The Memorial Sloan-Kettering experience and a literature review. J. Clin. Oncol., 16:3880-3889, 1998.

36. Heiner, J., Miraldi, F. D., Kallick, S., Makley, J., Smith-Mensah, W. H., Neely, J., Cheung, N. K. V. In vivo targeting of GD2 specific monoclonal antibody in human osteogenic sarcoma xenografts. Cancer Res., 47:5377-5381, 1987.

37. Chang, H. R., Cordon-Cardo, C., Houghton, A. N., Cheung, N. K. V., Casper, E. S., Brennan, M. F. Expression of disialogangliosides GD2 and GD3 by human soft tissue sarcomas. Cancer, 70:633-638, 1992.

38. Shinoura, N., Dohi, T., Kondo, T., Yoshioka, M., Takakura, K., Oshima, M. Ganglioside composition and its relation to clinical data in brain tumors. Neurosurgery, 31:541-549, 1992.

39. Longee, D. C., Wikstrand, C. J., Mansson, J. E., He, X., Fuller, G. N., Bigner, S. H., Fredman, P., Svennerholm, L., Bigner, D. D. Disialoganglioside GD2 in human neuroectodermal tumor cell lines and gliomas. Acta Neuropathology, 82:45-54, 1991.

40. Saarinen, U. M., Sariola, H., Hovi, L. Recurrent disseminated retinoblastoma treated by high-dose chemotherapy, total body irradiation, and autologous bone marrow rescue. Am. J. Pediatr. Hematol/Oncol., 13:315-319, 1991.

41. Cheresh, D. A., Honsik, C. J., Staffileno, L. K., Jung, G., Reisfeld, R. A. Disialoganglioside GD3 on human melanoma serves as a relevant target antigen for monoclonal antibody-mediated tumor cytolysis. Proc. Natl. Acad. Sci. USA, 82:5155-5159, 1985.

42. Thurin, J., Thurin, M., Herlyn, M., Elder, D. E., Steplewski, Z., Clark, W. H., Koprowski, H. GD2 ganglioside biosynthesis is a distinct biochemical event in human melanoma tumor progression. FEB Letters, 208:17-22, 1986.

43. Grant, S. C., Kostacoglu, L., Kris, M. G., Yeh, S., Larson, S. M., Finn, R. D., Oettgen, H. F., Cheung, N. K. V. Radioimmunodetection of small-cell lung cancer using the anti-GD2 ganglioside monoclonal antibody 3F8: a pilot trial. Eur. J. Nucl. Med., 23:145-149, 1996.

Second Series of Experiments

GD2 Synthase: A New Molecular Marker for Detecting Neuroblastoma

Background:

Neuroblastomas (NB) almost ubiquitously express the ganglioside GD2. GD2 synthesis is dependent on the key enzyme GD2 synthase. Thus, GD2 synthase transcript may prove to be a potential molecular marker of NB.

Methods:

77 NB tumor tissues of all stages, 5 NB cell lines, and 26 normal bone marrows (EM) and peripheral blood (PBL), as well as 26 non-NB remission BM were analyzed for the expression of GD2 synthase by a highly sensitive RT-PCR and chemiluminescence detection. 152 NB BMs were tested and comparisons were made amongst three independent detection techniques, namely GD2 synthase RT-PCR, immunofluorescence (IF), and histology (HIST).

Results:

GD2 synthase transcript was present in 5/5 cell lines and 77/77 tumors tested. Among 116 marrows that were positive by at least one of the three methods, 78% was detectable by GD2 synthase, 68% by IF, and 46% by HIST. 76% of positive BMs that were obtained during treatment and follow-up had GD2 synthase expression, whereas only 29% were HIST positive. Correlation between RT-PCR and IF was high (p=0.001) and positivity by at least 2 out of 3 methods at time of chemotherapy was strongly associated with adverse outcome (p=0.01). Serial samples (n=28) in five patients demonstrated close agreement between RT-PCR and their disease status.

Conclusions:

Molecular detection of GD2 synthase transcript in NB marrows may have potential value in detecting rare tumor cells.

Neuroblastoma (NB) shows a wide range of clinical behavior, ranging from localized tumors that can be cured with no treatment other than radical surgery, to metastatic aggressive tumors that tend to relapse despite intensive therapy. Marrow metastasis is a strong prognostic predictor of clinical outcome, and bone marrow remission is one criterion for judging the efficacy of treatment methods, as well as a requisite prior to autologous marrow transplantation, or termination of therapy. Unfortunately, when marrow disease is microscopic, standard sampling by histology shows limited sensitivity.^{1,2} While immunofluorescence can detect one tumor cell in 10⁵ nucleated cells,^{3,4} molecular detection of tumor-associated gene products by RT-PCR may improve the detection of minimal residual disease, whether in bone marrow (BM) or in peripheral blood (PBL). Several molecular markers have been tested in neuroblastoma. Tyrosine hydroxylase, the first enzyme in the pathway of catecholamine biosynthesis, seems to be a specific and sensitive marker, while PGP 9.5, an ubiquitin carboxy-terminal hydrolase, has been shown to be detectable in some normal BM and PBL.^{5,6} of increasing interest are families of genes, namely GAGE1,⁷ MAGE,⁸ and BAGE.⁹ They encode distinct tumor-associated antigens expressed in human tumors of different histological types, but are silent in normal adult tissues (except for placenta and testis). These genes are frequently expressed in NB,¹⁰⁻¹³ and GAGE1 positivity at time of minimal residual disease (MRD) has been shown to be a strong predictor of clinical outcome.¹⁴

GD2 synthesis is dependent on a key enzyme β1,4-N-acetyl-galactosaminyltransferase (GD2/GM2 synthase, EC 2.4.1.92) which catalyzes the transfer of 1,4-N-acetylgalactosamine to the precursor gangliosides GD3/GM3, respectively.¹⁵ In this report, the enzyme will be termed GD2 synthase for clarity, and not GD2/GM2 synthase. GD2 synthase is abundantly expressed in normal brain tissue of vertebrates¹⁶, and seems to be the key enzyme that controls the balance between expression of simple and complex gangliosides at the cell surface.¹⁷ This gene was assigned to human chromosome 12q13.3,⁵ and consists of at least 11 exons that span >8 kb. The coding region is located in exons 2-11, and 3 different transcription initiation sites have been identified. The enzyme is a 561-amino acid protein with a molecular mass of 61,001.89 daltons. This protein has an organization similar to that of known glycosyltransferases (type II transmembrane proteins). There is a single hydrophobic segment near the amino-terminus that is comprised of 18 amino acids and is flanked by basic residues This putative signal-anchor sequence would place 536 amino acids within the Golgi lumen and 7 residues within the cytosolic compartment.¹⁸

Besides neuroblastoma, the level of GD2 synthase activity is generally high in other neuroectoderm-derived tumor cells like malignant melanoma, as well as in adult T cell leukemia, and some colon and gastric cancer.¹⁹ In NB, the choice of GD2 synthase transcript as a potential molecular marker is particularly attractive, since almost all NB ubiquitously express the ganglioside GD2,²⁰ and likely the GD2 synthase transcript. GD2 density on neuroblastoma cells is high (5-10×10⁶ molecules per cell)²¹ and this antigen is rarely lost after anti-GD2 therapy.²²

To date, there has been only one report on the detection of GD2 synthase transcript in melanoma patients, showing a close correlation between tumor burden and disease progression.²³ The objective of this study was to determine the expression of GD2 synthase by RT-PCR and a sensitive chemiluminescent detection technique in NB tumors of all stages. Upon examining a panel of normal bone marrows and blood, its utility as a molecular marker in terms of tumor cell detection in 152 bone marrows (BM) from 100 NB patients was tested. These findings were compared with two established techniques, namely histology and immunofluorescence. Serial samples from individual patients were also examined and correlated with their clinical status.

Patients and Methods

Patients.

Patients with neuroblastoma evaluated at Memorial Sloan-Kettering Cancer Center were diagnosed and staged in accordance with the International Neuroblastoma Staging System.²⁴ Serial bone marrows were obtained as part of disease evaluation while the patient was being treated, with approval by the institutional review board of Memorial Hospital and informed consent from the patients and/or guardians. Each histological examination generally consisted of six samplings (two biopsies: right and left posterior iliac crest; and four aspirates: right and left anterior iliac crest, right and left posterior iliac crest obtained from six different sites of the iliac crests). Details of the procedure were previously described.²

Immunofluorescence.

Freshly collected heparinized bone marrow pooled from four aspiration sites was separated by ficoll centrifugation. Mononucleated cells were incubated with a panel of anti-GD2 monoclonal antibodies, followed by a reaction with a fluoresceinated anti-mouse IgG+IgM antibody. GD2-positive tumor cells were examined and enumerated using a fluorescent microscope by a trained technician. Quantitation was expressed in GD2 positive cells over total number of mononuclear cells in each counting chamber.²

Cell Lines.

Five NB cell lines were tested for GD2 synthase expression. 55N and 5S were provided by Dr. J. Biedler (Sloan-Kettering Institute, NY, N.Y.); LAN-1 was from Dr. R. Seeger (Children's Hospital of Los Angeles, Los Angeles, Calif.); NMB7 from Dr. S. K. Liao of McMasters University (Hamilton, Ontario, Canada); MSK-JD was established at MSKCC.

mRNA Extraction and cDNA Synthesis.

Cryopreserved bone marrow mononuclear cells were used. Total mRNAs were extracted and reverse transcription performed as previously described.¹⁰ Samples were coded for the molecular study.

PCR.

The oligonucleotides used as PCR primers for detecting GD2 synthase were previously described:²³ sense primer 5′-CCAACTCAACAGGCAACTAC-3′ and anti-sense primer 5′-GATCATAACGGAGGAAGGTC-3′. They were 5-end labeled with biotin (Integrated DNA Technologies, Coralville, Iowa). The specific PCR product was 230 bp. The quality of mRNA was checked by performing PCR using primers specific for human β2-microglobulin, namely 5′-CTCGCGCTACTCTCTCTTTCTGG-3′, and 5′-GCTTACATGTCTCGATCCCACTTAA-3′, obtaining an amplified DNA fragment of 333 bp.

PCR mixture contained 2.5 ul of 10×PCR buffer (100 mM Tris-HCl (pH 8.3), 500 mM KCl), 2.5 ul of 25 mM MgCl₂, 0.2 ul of 25 mM dNTPs, 0.1 ul of AmpliTaq Gold DNA polymerase (SU/ul Applied Biosystems, Foster City, Calif.), 0.1 ul of each oligonucleotide primer (50 uM), and 1.5 ul of cDNA. Nanopure H₂O was added to achieve a total volume of 25 ul. Each set of samples included a positive control (NB cell line LAN-1) and a negative control (PCR mixture without template). GD2 synthase primer-dimers formation was overcome by first heating the primers at 95° C. for 10 min before adding them to the mixture. PCR was performed in a Hybaid thermal cycler (Franklin, Mass.). PCR condition was set up as follows: 1 cycle of denaturation at 95° C. for 12 min, then 35 cycles of at 95° C., 65° C. and 72° C. for 1 min each before a final extension at 72° C. for 10 min. Genomic contamination was not amplified in this PCR reaction. Chemiluminescent detection of PCR products after electrophoresis was performed as previously described.¹⁰

### Results

Limit of Detection of GD2 Synthase RT-PCR

A cell spiking experiment was carried out to assess the sensitivity of this technique. LAN-1 cells were added to normal marrow mononuclear cells ranging from 1 to 10⁴ tumor cells/10⁶ BM cells. The negative control was one million normal marrow cells. Total mRNAs were extracted and RT-PCR performed to detect GD2 synthase expression. The limit of detection was 1 tumor cell in 10⁶ normal BM cells (FIG. 6). No RT-PCR product was detected in the negative control.

GD2 Synthase Expression in Cell Lines, Tumors, BM, and PBL (Table 4)

GD2 synthase mRNA was highly expressed in 5/5 GD2 positive NB cell lines, namely 55N, 5S, LAN-1, NMB7 and MSK-JD. They were detectable by ethidium bromide (EB) staining. Seventy-seven frozen NB tumors, including 12 stage 1, 12 stage 2, 12 stage 3, 11 stage 4S and 30 stage 4, were all positive by chemiluminescence, while only a few samples were detectable by EB alone. In order to confirm the potential use of GD2 synthase as a molecular marker, 12 normal BM, 14 normal PBL and 26 non-NB remission BM were examined. None of them was positive for GD2 synthase by chemiluminescence.

TABLE 4
GD2 synthase expression in cell lines, tumors,
bone marrows, and peripheral blood

| | Positivity in GD2 synthase expression |
|---|---|
| NB cell lines | 5/5 |
| NB tumors | 77/77 |
| Stage 1 | 12/12 |
| Stage 2 | 12/12 |
| Stage 3 | 12/12 |
| Stage 4S | 11/11 |
| Stage 4 | 30/30 |
| Normal bone marrow | 0/12 |
| Normal peripheral blood | 0/14 |
| Non-NB remission BM | 0/26 |

Detection of NB Cells in the BMs by Histology, IF, and GD2 Synthase RT-PCR

One hundred and fifty-two BM from 100 NB patients, 46 obtained at the time of diagnosis or relapse, 69 while on treatment, and 37 during follow-up, were studied. There was a high degree of concordance between molecular detection of GD2 synthase mRNA and immunofluorescence (IF), which measured the number of GD2-positive cells by a panel of anti-GD2 monoclonal antibodies (p=0.001). 57 samples were positive and 40 were negative by both methods (Table 5).

TABLE 5
Concordance in molecular versus immunologic detection
of GD2 in the bone marrow samples

| | Immunofluorescence | | |
|---|---|---|---|
| GD2 synthase RT-PCR | Positive | Negative | Total |
| Positive | 57 | 33 | 90 |
| Negative | 22 | 40 | 62 |
| Total | 79 | 73 | 152. |

P = 0.001

In this series, marrow detection by RT-PCR was compared to IF and histology. Table 6 tabulates BM positivity by each of these three detection methods according to sampling time. Negative detection was defined as negative by all three techniques. Among the 36 samples found to be negative by all 3 detection methods, only 5 were from time of diagnosis and relapse. Among the 116 positive marrows, 90/116 (78%) was detectable by GD2 synthase RT-PCR, 79/116 (68%) by IF, and 53/116 (46%) by histology. Interestingly, among positive marrows obtained during treatment and follow-up, 76% (57/75) had GD2 synthase expression, 63% (47/75) were IF positive, whereas only 29% (22/75) were histology positive.

TABLE 6
BM positivity by each of three detection methods versus
time of sampling

| | | | BM positivity | | |
|---|---|---|---|---|---|
| | Total | | Individual tests | | |
| Sample timing | n | +^{a} | HIST+ | IF+ | GD2 synthase+ |
| At diagnosis | 46 | 41 | 31 | 32 | 33 |
| & relapse | | | | | |
| During treatment | 106 | 75 | 22 | 47 | 57 |
| & follow-up | | | | | |
| Total | 152 | 116 | 53 | 79 | 90 |

^{a}Total+, positive by >1 of 3 tests.
^{b}HIST = histology

Association Between Concomitant Marrow Positivity and Patient Survival Status

Agreement among independent methods would increase the credibility of positive findings, especially from patients who were on treatment, or in apparent clinical remission, when their marrows likely had very few tumor cells. As shown in Table 7a, amongst patients on chemotherapy who had their marrows tested for this study, those who had positive marrows by 2 or 3 tests eventually died of the disease. Relationship between survival status and concomitant marrow positivity was statistically significant (p=0.01). A similar association between clinical outcome and positivity was also observed in patients whose marrows were sampled during their follow-up (Table 7b). Even though statistical significance has not been reached, among the 24 patients alive, 13 had negative marrows, and 6 were positive by only one test.

TABLE 7a
Association between positive marrows sampled when
patients were on chemotherapy and survival status

| | Marrow positivity detected by | | |
|---|---|---|---|
| Survival status | <1 test | >2 tests | Total patients |
| Alive | 7 | 2 | 9 |
| Dead | 5 | 14 | 19 |
| | 12 | 16 | 28 |

p = 0.010

TABLE 7b
Association between positive marrows sampled when
patients were on follow-up and survival status

| | Marrow positivity detected by | | |
|---|---|---|---|
| Survival status | <1 test | >2 tests | Total patients |
| Alive | 19 | 5 | 24 |
| Dead | 4 | 4 | 8 |
| | 23 | 9 | 32 |

p = 0.112

Detection of Marrow Disease by Histology, IF, and GD2 Synthase RT-PCR in Serial Samples

Serial marrows from 5 stage 4 NB patients (n=28) during the course of their treatment (chemotherapy, radiation, and immunotherapy) and follow-up were studied. Results are shown in Table 8. Detection by GD2 synthase transcript agreed with the findings by IF and histology in most cases. Patient #1 and #2 had marrows which were repeatedly negative at different sampling times are still alive and disease-free. Patient #3, #4 and #5 had marrow disease during treatment and follow-up detectable by means of histology, IF, and GD2 synthase RT-PCR. They all succumbed to NB and died.

### Discussion

Detection of occult NB cells in the BM has important therapeutic and prognostic implications, since BM disease is associated with unfavorable outcomes in NB.^{25,26} Sensitive methods are needed for the detection of very low levels of tumor cells in the BM of patients when they are on treatment or after the termination of therapy. Standard techniques, like histological examinations by Hematoxylin-Eosin stain of marrow biopsies and Wright-Giemsa stain of marrow aspirates, provide. good sensitivity at the time of diagnosis and relapse, when gross disease is present. However, they fail to detect minimal residual disease, even when the number of sites sampled was increased.² By using a panel of anti-GD2 antibodies, immunofluoresecence has proven to be a useful and important technique in detecting and quantifying NB cells in the BM.^{2,27} However, only freshly collected samples can be used, since archived or cryopreserved samples are unable to give reproducible results. Its limit of detection is 1 tumor cell in 10⁵ nucleated cells.^{1,20} Detecting residual NB cells by RT-PCR shows a higher sensitivity (1 tumor cell/10⁶ normal cells) using markers including tyrosine hydroxylase,²⁸ GAGE1,¹⁰ and in this report, GD2 synthase. Furthermore, RT-PCR can be performed on cryopreserved cells, and experiments can be repeated multiple times and for multiple markers.

In this study, using a very sensitive chemiluminescent detection method, GD2 synthase expression was detectable in all NB tissues of every stage, whereas a small number of samples were positive by ethidium bromide staining. The use of chemiluminescence to detect biotin-labeled PCR products has improved sensitivity dramatically¹⁰ without the need of nested PCR methods, which is often affected by the high number of false positives, even amongst samples from normal volunteers.^{5,29} The uniform GD2 synthase expression of NB tumors reflects the homogeneous presence of the antigen GD2. This potential molecular marker was found to have high specificity, since none of the normal BM or non-NB remission BM or normal PBL tested for the gene expression was detectable by chemiluminescence. This level of specificity gives GD2 synthase unique possibilities as a tumor marker for the detection of rare neuroblasts. Since GD2 is expressed by other malignant tumors, such as osteosarcoma,³⁰ melanoma,³¹ retinoblastoma,³² and some brain tumors,³³ the use of GD2 synthase as molecular marker may have broader clinical applications.

As a proof of principle, 152 BM from 100 NB patients were studied. Concordance between molecular detection of GD2 synthase and immunofluorescent detection of GD2 was high (p=0.001) (Table 5). This finding suggests that the level of GD2 synthase transcript was relatively uniform among tumor cells in the bone marrow. Furthermore, at time of diagnosis and relapse, frequency of GD2 synthase detection in marrow was comparable to that of tumor detection by histology or IF. During time of treatment and follow-up, frequency of BM positivity by GD2 synthase RT-PCR and IF was comparable, which was much higher than that by histology. This again demonstrates that histology can underestimate the prevalence of marrow involvement, especially at the time of minimal residual disease.

By using multiple independent detection techniques, namely histology, IF and RT-PCR, concomitant positivity of 2 to 3 methods strengthens the credibility of a positive finding, pointing to the presence of occult microscopic disease even when no gross disease is present.³⁴ In this retrospective analysis, survival status was strongly correlated with negativity of three concomitant tests, whether evaluated during induction chemotherapy or during follow-up (Table 7). Prognostic significance and sample timing deserve further investigation.

Serial samples (n=28) obtained from five stage 4 NB patients during the course of their treatment and follow-up were also studied (Table 8). Three patients with subsequent positive findings by IF and GD2 synthase RT-PCR succumbed and died of the disease. The other two patients whose BMs were repeatedly negative at different sampling times are still alive and disease-free. Of note were the negative findings by RT-PCR at diagnosis of pt #1 and pt #4, as well as the positive GD2 synthase expression in pt #2 at follow-up. Absence of expression might likely be due to the poor quality of the particular mRNA samples. On the other hand, positive finding in pt #2 could be a false positive, since this sample was neither IF nor histology positive. On the other hand, PCR does have higher limit of detection than IF. Overall, this reflects the limitation in qualitative PCR that permits only a positive versus a negative finding. In contrast, molecular detection of GD2 synthase by real-time quantitative RT-PCR is anticipated to be potentially a superior addition in the management of NB. It will provide more information on disease status, quantifying tumor cells in the bone marrows during treatment or follow-up. Thus, in combination with the requisite histological examinations, and the quantitative and sensitive IF technique, molecular detection by GD2 synthase transcript will be an invaluable addition in detecting NB cells in the bone marrows, especially in expediting the detection of rare tumor cells when patients are in clinical remission.

TABLE 8
Detection of marrow disease by histology (HIST),
immunofluorescence (IF), and GD2 synthase RT-PCR of
five stage 4 neuroblastoma patients during the course of
treatment and follow-up

| | | Mo. from | | % GD2 + | GD2 Synthase |
|---|---|---|---|---|---|
| | Timing | diagnosis | HIST | cells by IF | by RT-PCR |
| Pt # 1 | At diagnosis | 0 | + | 0.108% | − |
| | On treatment | 6 | − | 0.004% | + |
| | On treatment | 12 | − | — | − |
| | Follow-up | 18 | − | — | − |
| | PF | 84 | | | |
| Pt # 2 | On treatment | 1 | − | — | − |
| | On treatment | 6 | − | — | − |
| | On treatment | 9 | − | 0.002% | − |
| | Follow-up/ | 13 | − | — | − |
| | Follow-up | 20 | − | — | + |
| | Follow-up | 27 | − | — | − |
| | Follow-up | 40 | − | — | + |
| | PF | 92 | | | |
| Pt # 3 | At diagnosis | 0 | + | 13.895% | + |
| | On treatment | 13 | − | — | − |
| | Follow-up | 20 | − | — | − |
| | Follow-up | 24 | − | — | + |
| | Relapse | 30 | + | 0.270% | + |
| | Relapse | 41 | + | 0.335% | + |
| | Dead | 50 | | | |
| Pt # 4 | On treatment | 1 | + | 0.021% | − |
| | On treatment | 6 | − | — | + |
| | Follow-up | 18 | − | — | − |
| | Relapse | 20 | − | — | + |
| | Relapse | 25 | + | 0.950% | + |
| | Relapse | 36 | + | 4.354% | + |
| | Dead | 40 | | | |
| Pt # 5 | At diagnosis | 0 | + | 10.950% | + |
| | On treatment | 3 | − | 0.003% | + |
| | On treatment | 15 | − | 0.001% | + |
| | Relapse | 18 | − | — | + |
| | Relapse | 21 | − | 0.063% | + |
| | Dead | 24 | | | |

− = negative,
<0.001% for IF
+ = positive
PF = progression free

### REFERENCES

1. Moss T J, Reynolds C P, Sather H N, Romansky S G, Hammond G D, Seeger R C. Prognostic value of immunocytologic detection of bone marrow metastases in neuroblastoma. N Engl J Med 1991; 324:219-226.

2. Cheung N K, Heller G, Kushner B H, Liu C, Cheung I Y. Detection of metastatic neuroblastoma in bone marrow: when is routine marrow histology insensitive? J Clin Oncol 1997; 15:2807-2817.

3. Osborne M P, Rosen P P. Detection and management of bone marrow micrometastases in breast cancer. Oncology 1994; 8:25-42.

4. Pantel K. Detection of minimal disease in patients with solid tumors. J Hematother 1996; 5:359-367.

5. Mattano L A, Moss T J, Emerson S G. Sensitive detection of rare circulating neuroblastoma cells by the reverse transcriptase-polymerase chain reaction. Cancer Res 1992; 52:4701-4705.

6. Gilbert J, Norris M D, Marshall G M, Haber M. Low specificity of PGP9.5 expression for detection of micrometastatic neuroblastoma. Br J Cancer 1997; 75:1779-1781.

7. van den Eynde B, Peeters O, De Backer O, Gaugler B, Lucas S, Boon T. A new family of genes coding for an antigen recognized by autologous cytolytic T lymphocytes on a human melanoma. J Exp Med 1995; 182:689-698.

8. van der Bruggen P, Szikora J, Boel P, et al. Autologous cytolytic T lymphocytes recognize a MAGE-1 nonapeptide on melanomas expressing HLA-Cw 1601. Eur J Immunol 1994; 24:2134-2140.

9. Boel P, Wildmann C, Sensi M, et al. BAGE, a new gene encoding an antigen recognized on human melanomas by cytolytic T lymphocytes. Immunity 1995; 2:167-175.

10. Cheung I Y, Cheung N K V. Molecular detection of GAGE1 expression in peripheral blood and bone marrow: utility as a tumor marker for neuroblastoma. Clin Cancer Res 1997; 3:821-826.

11. Chen R L, Hong C M, Peters J, Matthay K K, Seeger R C. Frequent expression of MAGE and BAGE genes by neuroblastoma. Proc Am Assoc Cancer Res 1996; 37:463-464.

12. Ishida H, Matsumura T, Salgaller M L, Ohmizono Y, Kadono Y, Sawada T. MAGE-1 and MAGE-3 or -6 expression in neuroblastoma-related pediatric solid tumors. Int J Cancer 1996; 69:375-380.

13. Corrias M V, Scaruffi P, Occhino N, De Bernardi B, Tonini G P, Pistoia V. Expression of MAGE-1, MAGE-3 and MART-1 genes in neuroblastoma. Int J Cancer 1996; 69:403-407.

14. Cheung I Y, Chi S N, Cheung N K V. Prognostic significanceof GAGE1 detection in bone marrows on survival of patients with metastatic neuroblastoma. Med Ped Oncol 2000; 36:632-634.

15. Furukawa K, Soejima H, Niikawa N, Shiku H, Furukawa K. Genomic organization and chromosomal assignment of the human β1, 4-N-acetylgalactosaminyltransferase gene. J Biol Chem 1996; 271:20836-20844.

16. Yamamoto A, Haraguchi M, Yamashiro S, et al. Heterogeneity in the expression pattern of two ganglioside synthase genes during mouse brain development. J Neurochem 1996; 66:26-34.

17. Giraudo C G, Rosales Fritz V M, Maccioni H J F. GA2/GM2/GD2 synthase localizes to the trans-Golgi network of CHO-K1 cells. Biochem J 1999; 342:633-640.

18. Nagata Y, Yamashiro S, Yodoi J, Lloyd K O, Shiku H, Furukawa K. Expression cloning of β1,4N-acetylgalactosaminyltransferase cDNAs that determine the expression of GM2 and GD2 gangliosides. J Biol Chem 1992; 267:12082-12089.

19. Yuyama Y, Dohi T, Morita H, Furukawa K, Oshima M. Enhanced expression of GM2/GD2 synthase mRNA in human gastrointestinal cancer. Cancer 1995; 75:1273-1280.

20. Cheung N K, Von Hoff D D, Strandjord S, Coccia P F. Detection of neuroblastoma cells in bone marrow using GD2 specific monoclonal antibodies. J Clin Oncol 1986; 4:363-369.

21. Wu Z, Schwartz E, Seeger R C, Ladisch S. Expression of GD2 ganglioside by untreated primary human neuroblastomas. Cancer Res 1986; 46:440-443.

22. Kramer K, Gerald W, Kushner B H, Larson S M, Hameed M, Cheung N K V. Disialoganglioside GD2 loss following monoclonal antibody therapy is rare in neuroblastoma. Clin Cancer Res 1998; 4:2135-2139.

23. Kuo C T, Bostick P J, Irie R F, Morton D L, Conrad A J, Hoon D S B. Assessment of messenger RNA of β1,4-N-acetylgalactosaminyl transferase as a molecular marker for metastatic melanoma. Clin Cancer Res 1998; 4:411-418.

24. Brodeur G, Pritchard J, Berthold F, et al. Revisions of the international criteria for neuroblastoma diagnosis, staging and response to treatment. J Clin Oncol 1993; 11:1466-1477.

25. Hartman O, Valteau-Couanet D, Vassal G. et al. Prognostic factors in metastatic neuroblastoma in patients over 1 year of age treated with high-dose chemotherapy and stem cell transplantation: a multivariate analysis in 218 patients treated in a single institution. Bone Marrow Transpl 1999; 23:789-795.

26. Cotterill S J, Pearson A D J, Pritchard J, et al. Clinical prognostic factors in 1277 patients with neuroblastoma: results of the European Neuroblastoma Study Group “Survey” 1982-1992. Eur J Cancer 2000; 36:901-908.

27. Faulkner L B, Tintori V, Tamburini A, et al. High-sensitivity immunocytologic analysis of neuroblastoma cells in paired blood and marrow samples. J Hematother 1998; 7:361-366.

28. Miyajima Y, Kato K, Numata S I, Kudo K, Horibe K. Detection of neuroblastoma cells in bone marrow and peripheral blood at diagnosis by the reverse transcriptase-polymerase chain reaction for tyrosine hydroxylase mRNA. Cancer 1995; 75:2757-2761.

29. Ghossein R A, Rosai J. Polymerase chain reaction in the detection of micrometastases and circulating tumor cells. Cancer 1996; 78:10-16.

30. Heiner J, Miraldi F D, Kallick S, et al. In vivo targeting of GD2 specific monoclonal antibody in human osteogenic sarcoma xenografts. Cancer Res 1987; 47:5377-5381.

31. Cheresh D A, Honsik C J, Staffileno L K, Jung G, Reisfeld R A. Disialoganglioside GD3 on human melanoma serves as a relevant target antigen for monoclonal antibody-mediated tumor cytolysis. Proc Natl Acad Sci USA 1985; 82:5155-5159.

32. Saaarinen U M, Sariola H. Hovi L. Recurrent disseminated rtinoblastoma treated by high-dose chemotherapy, total body irradiation, and autologous bone marrow rescue. Am J Ped Hematol/Oncol 1991; 13:315-319.

33. Longee D C, Wikstrand C J, Mansson J E, et al. Disialoganglioside GD2 in human neuroectodermal tumor cell lines and gliomas. Acta Neuropathology 1991; 82:45-54.

34. Cheung I Y, Barber D, Cheung N K V. Detection of microscopic neuroblastoma in marrow by histology, immunocytology, and RT-PCR of multiple molecular markers. Clin Cancer Res 1998; 4:2801-2805.

Third Series of Experiments

Quantitation of GD2 Synthase mRNA by Real-Time Reverse Transcription-PCT: Clinical Utility in Evaluating Adjuvant Therapy in Neroblastoma

Purpose

Minimal residual disease (MRD) is one of the final hurdles to cancer cure. Since therapy (myeloablation/immunotherapy/differentiation) for MRD is applied at the time of clinical remission, objective surrogate markers are needed to gauge treatment efficacy.

Patients and Methods

Using quantitative RT-PCR of GD2 synthase mRNA, MRD response to anti-GD2 monoclonal antibody 3F8 adjuvant therapy was evaluated, namely one cycle of myeloablative radioimmunotherapy using ¹³¹I-3F8 plus one cycle of unlabeled 3F8 in 45 stage 4 neuroblastoma patients (newly diagnosed/without prior relapse) on the N7 protocol at Memorial Sloan-Kettering Cancer Center. The prognostic impact of MRD before and after adjuvant therapy on progression-free survival (PFS) and overall survival (OS) was also analyzed.

Results

Before 3F8 treatment, 24 of 45 patients were in complete remission (CR), 12 in very good partial remission (VGPR) and 9 in partial remission (PR), according to criteria from International Neuroblastoma Staging System plus ¹³¹I-3F8 scan. 71% had detectable tumor cells in marrow by real-time RT-PCR. Of the 32 positive patients, 20 became negative after therapy, with a 63% efficacy. When patients were stratified by CR/VGPR. versus PR, GD2 synthase positivity was prognostic when detected before 3F8-targeted therapy (PFS, p=0.045 and OS, p=0.010). Persistent marker positivity was also predictive of PFS (p=0.035) and OS (p=0.027). Patients who succumbed to the disease had four times the level of transcript than those who remain alive.

Conclusion

GD2 synthase mRNA is a useful surrogate marker for adjuvant treatment efficacy in neuroblastoma with strong prognostic potential.

One of the final hurdles to curing metastatic neuroblastoma is minimal residual disease (MRD), which escapes clinical detection leading to ultimate relapse and death. Finding an effective strategy to eradicate MRD has remained a formidable challenged.¹ Following induction, a common strategy employs myeloablative chemotherapy at the time of apparent clinical remission. This approach takes advantage of the linear dose-response relationship of individual chemotherapeutic drugs in specific tumors. Nevertheless, the value of such aggressive treatment, costly in both human and financial terms, remains debatable. A few randomized studies using myeloablative therapy with autologous stem cell rescue did demonstrate definite, although modest impact on outcome.²⁻⁵ On the other hand, biologics (immunotherapy and differentiation therapy) are emerging rapidly as alternative methods of adjuvant therapy at the time of clinical remission,^{2,6-9} appealing because of their generally mild toxicity. However, its true worth in eradicating MRD is difficult to assess, since ascertaining anti-tumor efficacy of individual agent will necessitate lengthy follow-up, waiting for clinical relapse or death.

When disease is macroscopic, objective response can be easily measured. Unfortunately, when disease is below the threshold of clinical detection, efficacy measurement is often imprecise, lacking in objectivity and hard to be verified. In fact, the inability to measure sub-clinical disease leaves no choice for cancer care professionals but to continue treatment (whether standard or experimental therapy) despite remission. This difficulty in quantifying MRD derives from the increasing demand of more stringent standards, where the unit of measure is an individual cell. Because of their low cell number, individual tumor cells escape detection by histological examination of BM biopsies or aspirates.^{10,11} In contrast, tumor-specific or tumor-associated mRNA or cDNA are signatures of viable tumor cells at the subcellular level, whether they travel alone or in company. This new level of standard is increasingly possible with RT-PCR and quantitative RT-PCR of specific gene transcripts serving as surrogate endpoints.

In this report, quantitative real-time RT-PCR of GD2 synthase mRNA was applied to evaluate treatment efficacy at the time of MRD, among stage 4 neuroblastoma (NB) patients undergoing N7 therapy at Memorial Sloan-Kettering Cancer Center (MSKCC).¹² GD2 synthase (β1,4-N-acetylgalactosaminyltransferase, EC 2.4.1.92) is the key enzyme required for GD2 synthesis.¹³ This transcript has been shown to have potential as a molecular marker of NB, in particular for the detection of rare NB cells.¹⁴⁻¹⁷ Recently, a highly sensitive and specific quantitative RT-PCR assay which measures GD2 synthase mRNA was validated. The transcript levels correlated well with the number of NB cells as measured by immunocytology.¹⁴ In the N7 protocol, anti-GD2 monoclonal antibody (MoAb) 3F8 was used to target radioimmunotherapy and immunotherapy to tumor cells when patients had achieved their minimal disease status. ¹³¹I-3F8 (hot antibody) directed myeloablative dose of radiation to residual NB, while unlabeled 3F8 (cold antibody) delivered complement-mediated^{18,19} and antibody-dependent cell-mediated cytotoxicity.²⁰⁻²² Disease response in the bone marrow to the combination of hot 3F8 plus one cycle of cold 3F8 among 45 patients; one patient did not receive cold 3F8 because of progressive disease. Furthermore, MRD was tested for any prognostic impact on patient survival.

### Patients and Methods

Patients Forty-five stage 4 NB patients on N7 protocol (36 newly diagnosed at MSKCC and 9 prior-treated without relapse), all >1 year of age at initial presentation, were the subjects of this study. The N7 protocol¹² utilized dose-intensive chemotherapy for induction, surgical resection and 2100 cGy hyperfractionated radiotherapy for local control, and for consolidating remission, targeted radioimmunotherapy with ¹³¹I-3F8 and immunotherapy with unlabeled 3F8. ¹³¹I-3F8 at 20 mCi/kg was myeloablative and required stem-cell support. Written informed consent was obtained from the patients and/or their parents in accordance with guidelines of the institutional review board of MSKCC.

Comprehensive extent-of-disease evaluation at MSKCC, in accordance to International Neuroblastoma Staging System (INSS) ,²³ included computed tomography (CT)/magnetic resonance imaging (MRI), ^{99m}Tc-bone scan, ¹³¹I- or 123I-metaiodobenylguanidine (MIBG) scan, urinary catecholamine metabolites measurements, as well as bone marrow studies evaluated by histochemical examinations of bilateral biopsy specimens and of aspirates from bilateral anterior and bilateral posterior iliac crests. Additionally, ¹³¹I-3F8 tumor imaging was carried out as previously described.²⁴ Staging evaluations carried out on patients before referral to MSKCC were uniformly less extensive.

Disease status was categorized according to the International Neuroblastoma Response Criteria:²³ complete response (CR), no evidence of disease; very good partial response (VGPR), primary mass reduced by 90%-99%, no evidence of distant disease except for skeletal residua, and catecholamines normal; partial response (PR), >50% decrease in measurable disease and ≦1 positive BM site; mixed response, >50% decrease of any lesion with <50% decrease in any other; no response, <50% decrease but <25% increase in any existing lesion; and progressive disease (PD), new lesion or >25% increase in an existing lesion.

Immunocytology was carried out as described previously.¹⁰ Freshly collected heparinized bone marrow pooled from four aspiration sites was separated by Ficoll centrifugation and mononucleated cells were incubated with a panel of anti-GD2 monoclonal antibodies, followed by a reaction with a fluoresceinated anti-mouse IgG antibody. GD2-positive tumor cells were examined and enumerated using a fluorescence microscope and quantitation was expressed as percentage of GD2-positive cells/total number of mononuclear cells in each counting chamber. Limit of detection was defined as ≦0.001% tumor cells.

Real-time quantitative RT-PCR was performed on cryopreserved bone marrows as previously described.²⁵ Relative quantitation of GD2 synthase mRNA was achieved in a multiplex PCR using the ABI Prism 7700 Sequence Detection System (Applied Biosystems, Foster City, Calif.). Details of the procedure were reported previously.¹⁴ For each unknown test sample, the amount of GD2 synthase and its endogenous reference glyceraldehyde-3-phosphate dehydrogenase (GAPDH) was determined from the respective standard curve. Dividing the GD2 synthase level by the GAPDH level resulted in a normalized GD2 synthase value. Based on the quantitation of a series of normal bone marrow and peripheral blood mononuclear cells, a normalized GD2 synthase value of ≦5 was defined as negative.

Statistical Analysis

Progression-free survival (PFS) and overall survival (OS) were measured in months from the beginning of N7 protocol. The probability of PFS and OS were estimated by the Kaplan-Meier method and survival comparison between groups by the log-rank test.

### Results

Disease Status and Response of Patients Prior to Radioimmunotherapy

Table 1 summarizes the disease status and clinical response of these patients at the time when their bone marrows were sampled prior to radioimmunotherapy for real-time RT-PCR. The median time from diagnosis was 9.2 months. Based on six modalities of detection, namely CT/MRI, ^{99m}Tc-bone scan, ¹³¹I- or ¹²³I-MIBG scan, urinary catecholamine metabolites measurements, bone marrow biopsy and aspirate examination, plus ¹³¹I-3F8 scan, 53% (24/45) of patients had no evidence of disease. Only five patients were positive in more than 3 detection modalites. In terms of clinical response, 80% (36/45) of the patients were in CR (n=24) or VGPR (n=12). Only 9 patients were in PR prior to ¹³¹I-3F8 radioimmunotherapy.

Efficacy During This Phase of Adjuvant Therapy

Even though these patients had good remission status as evidenced by the absence of disease detection in most of them, and none had positive marrow by histological examination, 32/45 of their marrows prior to treatment were tested positive by real-time quantitative RT-PCR of GD2 synthase mRNA (Table 2). Comparing to detection by immunocytology, nearly four times more samples were positive by RT-PCR. The difference between the two detection methods was much smaller for the post-treatment marrows. Twenty of the 32 RT-PCR positive patients had no detectable GD2 synthase transcript in their marrow after 3F8-targeted antibody treatment (one hot plus one cold 3F8), consistent with a marrow response of 63% (20/32) (Table 2).

Relationship Between Change in Transcript Level and Patient Survival

FIG. 7A illustrates the levels of GD2 synthase transcript when patients were stratified into 3 groups, namely those who had no detectable transcript (n=13), versus those whose marrows became negative after treatment (n=20), versus those whose marrows remained positive after this phase of immunotherapy. Patients who eventually died had transcript levels four times higher than those who remain alive (FIG. 7B). Additionally, among the 20 patients who eventually died, 45% (9/20) never had recurrence by histological examination in the bone marrow. They died from progressive disease or complications following relapse in the central nervous system (CNS) (n=4), CNS and lung (n=1), primary site (n=1), lymph node (n=1), bone (n=1) and treatment-related leukemia (n=1).

Prognostic Importance of GD2 Synthase-Positive Bone Marrows Prior to Radioimmunotherapy on Patient Survival

Bone marrows that tested positive for GD2 synthase transcript before hot antibody treatment were associated with adverse PFS (p=0.074) and OS (p=0.021), respectively. When stratified by clinical response (CR/VGPR versus PR) at the sampling time, patients with detectable GD2 synthase expression who were in partial remission at that time, fared even worse with p=0.045 for PFS (FIGS. 8A and 8B), and p=0.010 for OS (FIGS. 9A and 9B).

Prognostic Importance of GD2 Synthase-Positive Bone Marrows After Hot and One Cycle of Cold Antibody on Patient Survival

These patients had their marrows tested after the completion of hot antibody and one cycle of cold antibody. The median time from diagnosis was 16.7 months. Kaplan Meier analyses indicated that GD2 synthase positivity at the completion of this phase of adjuvant therapy was also associated with poor PFS (p=0.065) and OS (p=0.064), although with less statistical power when compared to marrows before 3F8-targeted therapy. Not surprisingly, patients whose bone marrows stayed positive for the GD2 synthase transcript had a significantly higher risk of disease progression (p=0.035, FIG. 10A) and death (p=0.027, FIG. 10B).

### Discussion

Accurate MRD measurements can provide objective and rapid surrogate endpoints in evaluating the efficacy of biologic/adjuvant therapies. It is hypothesized that MRD, being the likely final common pathway of inadequately treated or resistant cancer cells, will eventually be a dominant determinant of clinical outcome. After all, it is expected that the adverse effects of tumor biology will continue to be neutralized by increasingly effective treatment strategies. If MRD can indeed independently predict patient outcome, objective and measurable endpoint for biologic/adjuvant therapy may likely be realized.

In this study, it was striking and somewhat surprising to find that marrows drawn prior to adjuvant treatment were positive in over 70% of the patients by real-time RT-PCR, while only 20% of these patients had measurable disease by immunocytology, both assays identifying GD2-positive cells. Patients who succumbed to NB had transcript levels four times higher than those who remain alive. Upon the completion of one cycle of unlabeled 3F8 immunotherapy, 63% of the positive marrows became negative by real-time RT-PCR. This surrogate endpoint would indeed suggest that the adjuvant therapy was efficacious. More importantly, the question of ‘true’ efficacy must be addressed: does GD2 synthase mRNA positivity correlate with clinical outcome, specifically to the gold standard of patient survivals, both progression-free and overall survival?

In this phase of immunotherapy, GD2 synthase transcript was found to correlate with both PFS and OS with statistical significance. This was not so when immunocytology as a variable was analyzed. These Kaplan-Meier analyses generated several interesting observations. First of all, despite 71% marrow positivity prior to 3F8-targeted therapy, only 44% succumbed to their disease, strongly suggesting a potential efficacy of antibody-targeted adjuvant therapy on patient survival. Furthermore, 45% of the patients who eventually died did not have recurrence in the bone marrow. Soft tissue relapse such as lung, CNS, and t-AML caused their ultimate demise. Secondly, patients with persistently positive bone marrows detectable by RT-PCR before hot antibody treatment, after hot antibody, and after one cycle of cold antibody were more likely to progress and die from the disease. Fukuda et al reported that persistent tyrosine hydroxylase mRNA in the BM of stage 4 NB patients tested at more than 4 months after the start of chemotherapy was associated with poor prognosis.²⁶ Thus, achieving and maintaining tumor-free marrow during this consolidation could be critical for improving patient outcome. Of note were patients who, despite persistent positive marker in the marrow during this phase of adjuvant therapy, finally achieved molecular remission and remained disease free. They may have become long-term survivors because of the anti-NB activity of treatment with subsequent additional cycles of 3F8.

Thirdly, despite their apparent clinical remission by INSS criteria plus ¹³¹I-3F8 scan, patients who were GD2 synthase positive before 3F8-targeted immunotherapy had statistically poorer PFS and OS. This could be the result of either slow-responding or sub-clinical progressive disease. Our finding is in agreement with a much larger series of stage 4 NB patients in the CCG study, when increasing tumor cell content in the bone marrow as measured by immunocytology had an adverse effect on event free survival when tested at 12 weeks (3 cycles of chemotherapy), at bone marrow harvest, and at the end of induction.²⁷ It was not surprising that RT-PCR positivity was particularly adverse among patients who were in partial remission prior to adjuvant therapy. One could argue that improving the remission status before mega-therapy consolidation may be necessary in order to improve patient outcome. However, whether marker positivity was because of slow-responding versus progressive disease has important implications for future therapy design. While slow-responders may benefit from continued chemotherapy, progressive disease demands, at the very least, change in chemotherapeutic strategy.

Lastly, post-treatment marrow positivity was associated with poorer PFS and OS, though with less statistical power. This could be due to the impact of treatment overriding the influence of marrow relapse. Interestingly, positive GD2 synthase mRNA in the bone marrows tested at 24 months after diagnosis was strongly associated with PFS (p<0.005) and OS (p<0.001) among advanced stage NB patients diagnosed at >1 year of age initially treated with protocols N6 and N7.¹⁴ Similar conclusions were drawn when another molecular marker GAGE1 was tested in the same cohort.²⁸ This reflects that detection of residual tumor cells long after the end of therapy would render measurement of MRD in the bone marrow with greater prognostic importance.

Quantitation of GD2 synthase mRNA by real-time quantitative RT-PCR did prove to have clinical relevance in evaluating adjuvant therapy in NB (1) by detecting sub-clinical level of tumor cells, and (2) by being predictive of long term patient outcome. This study, albeit small in size (n=45), included only newly diagnosed/without prior relapse stage 4 patients treated in a single institution under the same protocol. Our survival analyses seemed to suggest the need to ensure patients achieve both marrow remission by this technique (i.e. the absence of molecular relapse), as well as clinical remission (CR/VGPR) before the onset of adjuvant therapy, in order to achieve long-term cure. The question of MRD and clinical relevance may potentially be resolved upon the completion of the Children's oncology Group phase III trials on the detection of MRD among high-risk NB patients performed at various times during therapy.²⁹

TABLE 1
Disease status and clinical response of 45 (newly
diagnosed/without prior relapse) stage 4 NB patients
before ¹³¹I-3F8 radioimmunotherapy

| | Number of patients |
|---|---|
| Evidence of disease* | |
| 0/6 | 24 |
| 1/6 | 8 |
| 2/6 | 8 |
| 3/6 | 3 |
| 4/6 | 2 |
| Clinical response** | |
| CR | 24 |
| VGPR | 12 |
| PR | 9 |

*6 modalities of disease detection included CT/MRI, ^{99m}Tc-bone scan, ¹³¹I- or ¹²³I-MIBG scan, urinary HVA/VMA (homovanillic acid/vanillylmandelic acid), corrected for age and creatinine, bone marrow biopsy and aspirate examination, plus ¹³¹I-3F8 scan.
**clinical response according to INSS criteria as described in Patients and Methods section.

TABLE 2
Tumor cell detection in the bone marrow by real-time
quantitative RT-PCR versus immunocytology and
histology during immunotherapy*

| | Before hot antibody | | After hot + 1 cycle cold | |
|---|---|---|---|---|
| Positivity by | No. of patients | (% Pos) | No. of patients | (% Pos) |
| GD2 synthase | 32/45 | (71.1%) | 12/45 | (26.7%) |
| Real-time RT-PCR | | | | |
| Immunocytology | 8/41 | (19.5%) | 9/44 | (20.5%) |
| Histology | 0/45 | (0%) | 4/45 | (8.9%) |

*hot = ¹³¹I-3F8;
cold = unlabeled 3F8
GD2 Synthase/GAPDH ≦ 5 was defined as negative.

### REFERENCES

1. Cheung N K, Kushner B H, LaQuaglia M P, et al: Treatment of advanced stage neuroblastoma, in Raghavan D, Scher H I, Leibel S A, et al (eds): Principles and Practice of Genitourinary Oncology. Philadelphia, Lippincott-Raven, 1997, pp 1101-1111

2. Matthay K K, Villablanca J G, Seeger R C, et al: Treatment of high-risk neuroblastoma with intensive chemotherapy, radiotherapy, autologous bone marrow transplantation, and 13-cis-retinoic acid. N Engl J Med 341:1165-1173, 1999

3. Frappaz D, Michon J, Coze C, et al: LMCE3 treatment strategy: results in 99 consecutively diagnosed stage 4 neuroblastomas in children older than 1 year at diagnosis. J Clin Oncol 18:468-476, 2000

4. Pole J G, Casper J, Elfenbein G. et al: High-dose chemoradiotherapy supported by marrow infusions for advanced neuroblastoma: a Pediatric Oncology Group Study. J Clin Oncol 9:152-158, 1991

5. Ladenstein R, Philip T, Lasset C, et al: Multivariate analysis of risk factors in stage 4 neuroblastoma patients over the age of one year treated with megatherapy and stem-cell transplantation: a report from the European Bone Marrow Transplantation Solid Tumor Registry. J Clin Oncol 16:953-965, 1998

6. Cheung N K V, Kushner B H, Yeh S J, et al: 3F8 monoclonal antibody treatment of patients with stage IV neuroblastoma: A phase II Study. Int J Oncol 12:1299-1306, 1998

7. Cheung N K V, Kushner B H, Cheung I Y, et al: Anti-GD2 antibody treatment of minimal residual stage 4 neuroblastoma diagnosed at more than 1 year of age. J Clin Oncol 16:3053-3060, 1998

8. Kushner B H, Kramer K, Cheung N K V: Phase II trial of 3F8 monoclonal antibody and granulocyte macrophage colony-stimulating factor (GM-CSF) for neuroblastoma. J Clin Oncol, 2001

9. Yu A, Uttenreuther-Fischer M, Huang C-S, et al: Phase I trial of a human-mouse chimeric anti-disialoganglioside monoclonal antibody ch14.18 in patients with refractory neuroblastoma and osteosarcoma. J Clin Oncol 16:2169-2180, 1998

10. Cheung N K, Heller G, Kushner B H, et al: Detection of metastatic neuroblastoma in bone marrow: when is routine marrow histology insensitive? J Clin Oncol 15:2807-2817, 1997

11. Cheung I Y, Barber D, Cheung N K V: Detection of microscopic neuroblastoma in marrow by histology, immunocytology, and RT-PCR of multiple molecular markers. Clin Cancer Res 4:2801-2805, 1998

12. Cheung N K, Kushner B H, LaQuaglia M, et al: N7: A novel multi-modality therapy of high risk neuroblastoma (NB) in children diagnosed over 1 year of age. Med Pediatr Oncol 36:227-230, 2001

13. Furukawa K, Soejima H, Niikawa N, et al: Genomic organization and chromosomal assignment of the human β1, 4-N-acetylgalactosaminyltransferase gene. J Biol Chem 271:20836-20844, 1996

14. Cheung I Y, Cheung N K V: Quantitation of marrow disease in neuroblastoma by real-time reverse transcription-PCR. Clin Cancer Res 7:1698-1705, 2001

15. Lo Piccolo M S, Cheung N K V, Cheung I Y: GD2 Synthase: a new molecular marker for detecting neuroblastoma. Cancer 92:924-931, 2001

16. Hoon D S B, Kuo C T, Wen S, et al: Ganglioside GM2/GD2 synthetase mRNA, is a marker for detection of infrequent neuroblastoma cells in bone marrow. Am J Pathol 159:493-500, 2001

17. Cheung I Y, Lo Piccolo M S, Collins N, et al: Quantitation of GD2 synthase mRNA by real-time reverse transcription-PCR: Utility in bone marrow purging of neuroblastoma by anti-GD2 antibody 3F8. Cancer (in press)

18. Cheung N K V, Walter E I, Smith-Mensah W H, et al: Decay-accelerating factor protects human tumor cells from complement-mediated cytotoxicity in vitro. J Clin Invest 81:1122-1128, 1988

19. Chen S, Caragine T, Cheung N K, et al: Surface antigen expression and complement susceptibility of differentiated neuroblastoma clones. Am J Pathol 156:1085-1091, 2000

20. Kushner B H, Cheung N K: GM-CSF enhances 3F8 monoclonal antibody-dependent cellular cytotoxicity against human melanoma and neuroblastoma. Blood 73:1936-1941, 1989

21. Munn D H, Cheung N K: Interleukin-2 enhancement of monoclonal antibody-mediated cellular cytotoxicity (ADCC) against human melanoma. Cancer Res 47:6600-6605, 1987

22. Munn D H, Cheung N K: Phagocytosis of tumor cells by human monocytes cultured in recombinant macrophage colony-stimulating factor. J. Exp. Med. 172:231-237, 1990

23. Brodeur G, Pritchard J, Berthold F, et al: Revisions of the international criteria for neuroblastoma diagnosis, staging and response to treatment. J Clin Oncol 11:1466-1477, 1993

24. Yeh S D, Larson S M, Burch L, et al: Radioimmunodetection of neuroblastoma with iodine-131-3F8: Correlation with biopsy, iodine-131-Metaiodobenzylguanidine (MIBG) and standard diagnostic modalities. J Nucl Med 32:769-776, 1991

25. Cheung I Y, Cheung N K V: Molecular detection of GAGE1 expression in peripheral blood and bone marrow: utility as a tumor marker for neuroblastoma. Clin Cancer Res 3:821-826, 1997

26. Fukuda M, Miyajima Y, Miyashita Y, et al: Disease outcome may be predicted by molecular detection of minimal residual disease in bone marrow in advanced neuroblastoma: a pilot study. J Pediatr Hematol Oncol 23:10-13, 2001

27. Seeger R C, Reynolds C P, Gallego R, et al: Quantitative tumor cell content of bone marrow and blood as a predictor of outcome in stage IV neuroblastoma: a Children's Cancer Group study. J Clin Oncol 18:4067-4076, 2000

28. Cheung I Y, Chi S N, Cheung N K V: Prognostic significance of GAGE1 detection in bone marrows on survival of patients with metastatic neuroblastoma. Med Pediatr Oncol 35:632-634, 2000

29. Reynolds C P, Seeger R C: Detection of minimal residual disease in bone marrow during or after therapy as a prognostic marker for high-risk neuroblastoma. J Pediatr Hematol Oncol 23:150-152, 2001

Fourth Series of Experiment

Quantitation of GD2 Synthase mRNA by Real-Time Reverse Transcription-PCR: Utility in Bone Marrow Purging of Neroblastoma by Anti-Gd2 Antibody 3F8

Background

Antigen ganglioside GD2 is abundantly expressed on neuroblastoma (NB). Anti-GD2 monoclonal antibody (MoAb) 3F8 kills NB cells by complement-dependent cytotoxicity and antibody-dependent cellular cytotoxicity. Its utility in bone marrow (BM) purging is evaluated by a real-time reverse transcription-PCR (RT-PCR) assay to quantify the mRNA of GD2 synthase, the key enzyme in GD2 synthesis.

Methods

A pilot study (1990-1993) was carried out in 10 patients with relapsed/refractory stage 4 MB using MoAb 3F8 to purge tumor cells from harvested BM which had <5% tumor content by immunocytology (IF). Subsequently, 31 stage 4 NB patients who underwent treatment on the N7 protocol (1994-1999) had their remission BM purged by 3F8 prior to ¹³¹I-3F8 myeloablative radioimmunotherapy. GD2-positive tumor cells before and after purging were quantified by real-time quantitative RT-PCR of GD2 synthase.

Results

GD2 positivity by IF was found in 6/8 patients on the pilot study before purging, 5/6 became negative post-purging. Of 31 patients on the N7 protocol, even though pre-purge BM were negative by histology and IF, the more sensitive real-time quantitative RT-PCR detected GD2 synthase mRNA in 7 BM. Six of the 7 marrows became negative after 3F8 purging. Marker positivity prior to purging was statistically significant in predicting overall survival (p=0.04), but not progression-free survival (p=0.1). In vitro hematopoietic stem cell recovery and the median time to engraftment were acceptable.

Conclusion

Tumor cell depletion quantified by real-time RT-PCR demonstrated efficacy of MoAb 3F8 in bone marrow purging.

Autologous marrow/stem cell transplantation is an integral part of the curative strategy for refractory cancers like neuroblastoma (NB).¹ Clearly tumor-free harvests are of critical importance, since genetically marked tumor cells in autologous bone marrow were demonstrated to be present at site of disease relapse in NB and AML.^{2, 3} A variety of in vitro methods have been explored to purge tumor cells of diverse origin. Chemical purging utilizes chemotherapeutic agents such as 4-hydroperoxycyclophosphamide,⁴ and mafosfamide. ⁵ Immunomagnetic depletion of tumor cells exploits the tagging of tumor-reactive monoclonal antibody (MoAb) to antimouse IgG-coated magnetic polystyrene microspheres.⁶⁻⁸ Complement-mediated purging takes advantage of unique MoAb that can activate the complement cascade in sensitive tumors.^{9, 10} Photoradiation of tumor cells is another method for purging autologous bone marrow grafts.¹¹ More recently, selection of antigen CD34-positive stem cells has become an attractive method to deplete tumor cells from peripheral blood stem cells (PBSC) because this antigen is only expressed on early lymphohematopoietic stem cells and progenitor cells, and not on mature blood cells or on tumor cells.^{12, 13} A relatively recent development in bone marrow purging is the use of viral-directed enzyme prodrug.¹⁴ Adenovirus was utilized to deliver the cDNA encoding a rabbit liver carboxylesterase that activates the prodrug CPT-11 to its active form SN-38 in NB cell lines efficiently.

The choice of purging method takes into account many factors. Two crucial considerations are efficacy of purging (number of log tumor cell removal) and safety of purging (preservation of hematopoietic stem cells). Cost and ease of purging in terms of length of the procedure need also be considered. Purging methods using monoclonal antibody and chemicals are technically simple, whereas viral purging presents potential biohazard concerns, requiring more complex regulatory oversight. To date, there are no reports that compare the efficacy and safety among different purging techniques. Therefore, the relative impact of tumor purging in autologous stem cell transplantation in the overall strategy of cancer treatment remains uncertain. NB cells express the antigen ganglioside GD2 at high density, with little heterogeneity within tumors or among patients.^{15, 16} This antigen has limited distribution in normal human tissues and is not modulated from the cell surface when bound by antibodies. In addition, patients generally do not have enough soluble GD2 to interfere with anti-GD2 MoAb which include 3F8 (murine IgG3)¹⁷ and 14.G2a (murine IgG2a).¹⁸ Because NB cells lack complement inhibitory proteins such as decay-accelerating factor CD55¹⁹ and CD59,²⁰ they are highly susceptible to complement-dependent cytotoxicity (CDC)¹⁰ and antibody-dependent cellular cytotoxicity (ADCC).^{21, 22} MoAb 3F8 mediates CDC^{19, 20} and ADCC efficiently.²³⁻²⁵

GD2 synthase (β1, 4-N-acetylgalactosaminyltransferase, EC 2.4.1.92) is the key enzyme required for GD2 synthesis.²⁶ This transcript has been shown to have potential as a molecular marker of NB, in particular for the detection of rare neuroblastoma cells.²⁷⁻²⁹ Recently, a highly sensitive and specific quantitative RT-PCR assay to measure GD2 synthase mRNA, which correlates directly with tumor cell content was validated.²⁷ From 1990-1999, 41 marrow samples from patients with stage 4 NB were purged with anti-GD2 MoAb 3F8 at Memorial Sloan-Kettering Cancer Center (MSKCC). This report evaluated the utility of ex vivo 3F8 purging by quantifying residual GD2-positive NB by real-time RT-PCR of GD2 synthase mRNA as well as by immunocytology (IF). The prognostic importance of marker positivity was also explored.

### Patients and Methods

Patients

A pilot study (1990-1993) was carried out in 10 patients with relapsed/refractory stage 4 NB using MoAb 3F8 to purge tumor cells from harvested BM which had ≦5% tumor content by IF. Subsequently, 31 stage 4 NB patients who underwent the N7 protocol (1994-1999) had their remission BM purged by 3F8 prior to ¹³¹I-3F8 myeloablative radioinmunotherapy.³⁰ In brief, the N7 protocol utilized 7 cycles of dose-intensive chemotherapy for induction, surgical resection and 2100 cGy hyperfractionated radiotherapy for local control, and for consolidating remission, targeted radioimmunotherapy with 131I-3F8 and immunotherapy with unlabeled/unmodified 3F8. Written informed consent was obtained from the patients and/or their parents in accordance with guidelines of the institutional review board of MSKCC.

Bone Marrow Purging Procedure

Autologous bone marrow was harvested under general anesthesia from both anterior and posterior iliac crests. Marrow was filtered through coarse filters (0.3 mm mesh) and then transferred into blood bags. The amount of harvested marrow was at least 15-20 ml/kg of patient size. 60% of the harvested marrow was treated with monoclonal antibody 3F8 and 40% was used as a source of autologous plasma and was left untreated. A sample was taken for cell viability testing, clonogenic assays, and fluorescence microscopic examination for tumor cells. The rest of the marrow (60%) was purged as follows: Total marrow volume and nucleated cell count was measured. Sterile MoAb 3F8 was added at a final concentration of 10 ug/ml and was allowed to incubate with marrow for one hour at 37° C. with shaking. The specimen was washed twice at 10° C. in the presence of sterile 10% autologous plasma. A sample was taken for cell viability testing, clonogenic assays, and fluorescence microscopic examination for tumor cells. Both purged and untreated marrows were delivered to the autologous purging laboratory for cryopreservation.

Clonogenic Marrow Stem Cell Assays³¹

The following parameters were measured before and after purging: colony-forming units-granulocyte-macrophage (CFU-GM) colony count/10⁶ nucleated cells, burst-forming units-erythrocyte (BFU-E) colony count/10⁶ nucleated cells and % CD34-positive cells. The percent recovery of the total nucleated cell count (TNC)/kg of patient weight after purging was also recorded.

Immunocytology was carried on the pre- and post-purged bone marrows to determine log tumor kill as described previously.³² In brief, freshly collected heparinized bone marrow pooled from four aspiration sites was separated by Ficoll centrifugation and mononucleated cells were incubated with a panel of anti-GD2 monoclonal antibodies, followed by a reaction with a fluoresceinated anti-mouse IgG antibody. GD2-positive tumor cells were examined and enumerated using a fluorescence microscope and quantitation was expressed as percentage of GD2-positive cells/total number of mononuclear cells in each counting chamber. Limit of detection was defined as ≦0.001% tumor cells.

Real-time quantitative RT-PC was performed on cryopreserved bone marrows collected prior to and after purging, as well as on mononuclear cells seeded with varying concentrations of NB cell line LAN-1 in the presence/absence of MoAb 3F8. Relative quantitation of GD2 synthase mRNA was achieved in a multiplex PCR using the ABI Prism 7700 Sequence Detection System (Applied Biosystems, Foster City, Calif.). Details of the procedure were reported previously.²⁷ For each unknown test sample, the amount of GD2 synthase and its endogenous reference glyceraldehyde-3-phosphate dehydrogenase (GAPDH) was determined from the respective standard curve. Dividing the GD2 synthase level by the GAPDH level resulted in a normalized GD2 synthase value. Based on the quantitation of a series of normal bone marrow and peripheral blood mononuclear cells, a normalized GD2 synthase value of ≦5 was defined as negative.

Statistical Analysis

Progression-free survival (PFS) and overall survival (OS) were measured in months from the beginning of N7 protocol. The probability of PFS and OS were estimated by the Kaplan-Meier method and survival comparison between groups by the log-rank test.

### Results

Efficiency of Purging Using Marrow Spiked with NB Cell Line

It was previously shown that the number of GD2-positive cells by IF correlated with the mRNA levels of the gene transcript GD2 synthase as measured by quantitative real-time RT-PCR (r=0.96).²⁷ Moreover, RT-PCR is more sensitive than IF in tumor cell detection; the latter method is typically used as a criterion for marrow or stem cell harvesting. To validate that the GD2 synthase transcript could serve as a surrogate marker for purging efficacy, marrow mononuclear cells were seeded with NB cell line LAN-1 cells at concentrations of 0.5%, 1%, and 2% in the presence of autologous plasma. MoAb 3F8 was added to the experimental groups at 10 ug/ml. All samples were incubated at 37° C. for 1 hour with gentle shaking. To simulate the cryopreserved marrow samples, the cells were washed and cryopreserved in 10% DMSO plus 10% fetal calf serum at −80° C. for one week. Upon thawing, mRNA was extracted from the cell pellet, followed by reverse transcription and real-time quantitative PCR of molecular marker GD2 synthase. A 10-fold decrease in GD2 synthase level was achieved at each spiked concentration of LAN-1 cells (FIG. 11).

Marrow Purging With 3F8 in Patients with Relapsed/Refractory NB

Ten patients with relapsed/refractory stage 4 NB whose harvested marrow had ≦5% tumor by IF were the subjects of this pilot purging study (1990-93). IF was not evaluated in two patients. When tested by IF, which had a limit of detection of 0.001%, 6/8 patients had marrow disease prior to purging, ranging from 0.002% to 0.255% (Table 1). After 3F8 purging, bone marrows of 5/6 patients became negative (Table 1). Post-purge marrow of patient #3 had a 73-fold depletion of tumor cells, even though residual tumor cells were still detectable. When assayed for marrow progenitors, there was a 19% decrease in total viable nucleated cell count as well as BFU-E activity, while CFU-GM colony count was unaffected (Table 2).

Marrow Purging with 3F8 in NB Patients in Clinical Remission

Thirty-one patients with stage 4 NB treated with the N7 protocol (1994-99) had their remission marrows harvested for ex vivo 3F8 purging. Prior to bone marrow harvest, 22 patients had 5 or more cycles of induction chemotherapy. Eight patients had 3 to 4 cycles, while one patient who had no marrow disease at diagnosis was harvested after 2 cycles. All 31 marrows were negative by histology, 3 of the 31 were low positive (0.002%-0.004%) by IF. When assayed by real-time quantitative RT-PCR, marrows of 7 patients were positive for GD2 synthase mRNA prior to purging (Table 3). Six became negative after purging. In one patient, the relative tumor content did not change following 3F8 purging. As shown in Table 2, among the N7 patients, there was a 12% loss in total nucleated cell count and in CD34+ cells. BFU-E colony count dropped by 14%, while CFU-GM colony count had a 5% increase. Twenty-four of 31 patients subsequently proceeded with myeloablative ¹³¹I-3F8 radioimmunotherapy followed by infusion of purged marrow. The median time to engraftment was 19 days to reach an absolute neutrophil count greater than 500/uL; 49 days to reach a self-sustaining platelet counts greater than 20,000/uL, and 102 days to reach platelet counts greater than 50,000/uL.

Prognostic Significance of GD2 Synthase Positivity on Survival

In this cohort of 31 stage 4 NB patients who had their remission BM purged by MoAb 3F8 prior to ¹¹³I-3F8 myeloablative radioimmunotherapy, there was a statistical difference in the overall survival among patients whose pre-purge marrows were positive for the GD2 synthase transcript, versus those whose marrows were negative (p=0.04, FIG. 12). However, progression-free survival did not reach statistical significance (p=0.10, FIG. 13). Since all but one marrow were negative after purging, the prognostic importance of purged marrows and its cleanliness was not evaluable. In total, overall survival in this cohort was 52% (16/31), at 34 months of median followup.

### Discussion

In order to establish useful ex vivo purging techniques for patients, many investigators have reported in vitro experiments to demonstrate purging efficiency. Varying concentrations of tumor cells were seeded in normal bone marrow/peripheral blood mononuclear cells to achieve 2 to 4 logs of tumor cell depletion. Detection methods ranged from the use of fluorescent nuclear dye Hoechst 33342.⁶³³ immunocytology,⁷ to RT-PCR.^{8,34} In viral-directed enzyme prodrug technique, even 10% tumor contamination was depleted as evidenced by RT-PCR.¹⁴ Unmodified MoAb 3F8 was effective in purging remission marrows seeded with up to 5% neuroblastoma cells, with a 2-3 log efficiency when assayed by counting fluorescent cells already quenched by trypan blue, with no detectable adverse effects on in vitro marrow stem cell growth.³³

Ex vivo use of MoAb for purging has primarily been based on immunomagnetic techniques, requiring specialized beads, reagents, elution systems and software. Here, it was demonstrated that the utility of one single purging agent 3F8 without the requisite addition of heterologous complement, since 3F8 mediates ADCC and CDC efficiently by using autologous cells and plasma, respectively. This was a cost-effective technique requiring only a one-hour incubation at 37° C. and two centrifugations. Among patients tested, tumor cell depletion was documented by IF which enumerated GD2-positive cells by indirect immunofluorescence, as well as the more sensitive real-time quantitative RT-PCR of GD2 synthase mRNA. Our laboratory has previously shown that this gene transcript has clinical utility as a molecular marker of minimal residual disease.^{27,28} The spiking experiment reported here is the first demonstration that GD2 synthase mRNA in NB cells, when damaged by MoAb 3F8-mediated CDC and ADCC, became degraded and was no longer measurable by RT-PCR. In vitro hematopoietic stem cell recovery in both the pilot and N7 series was within acceptable range. The median time to engraftment for our N7 patients was comparable to that reported previously on our stage 4 NB patients undergoing myeloablative combination chemotherapy whose autologous marrows were purged with 4-hydroperoxycyclophosphamide.³⁵

Ex vivo purging is based on the rationale that thorough removal of tumor cells from harvested marrows/stem cells will hopefully prevent relapse caused by the reinfusion of tumorigenic cells during autologous bone marrow/stem cell transplantation. Moreover, the quality of these re-infused cells is crucial in sustaining stem cell reconstitution. As the sensitivity of detection techniques improves, residual NB after purging is frequently detectable following ex vivo purging.^{12, 36-38} In general, the mean log(10) tumor reduction ranged from 1 to 3, depending on the method of detection. The clinical significance of a small number of tumor cells in the graft is not known.

In a study of 17 stage 4 NB patients, Lode et al found a better although non-significant event-free survival in patients transplanted with CD34+ selected peripheral blood stem cells which had no detectable NB cells as evidenced by tyrosine hydroxylase RT-PCR and anti-GD2 immunocytochemistry.³⁸ Another series of 18 poor-risk NB patients with initial marrow disease had in vivo marrow purging by conventional chemotherapy.³⁹ Based on detection by histological examinations and immunofluorescence using an anti-GD2 antibody, Saarinen et al reported that the probability of 4-year disease-free survival was 5/5 for patients with successful purging, 5/8 for patients who eventually succeeded in purging, and 0/5 for those whose purging remained unsuccessful (p<0.001).

Our cohort of 31 stage 4 patients all underwent identical treatment on protocol N7. Three pre-purge marrows (10%) were positive by IF, whereas 23% were positive by RT-PCR. Ex vivo purging with 3F8 was quite effective in depleting GD2-positive tumor cells, since 6/7 positive marrows became negative after purging. Interestingly, patients whose pre-purge (n=7) marrows were positive by RT-PCR had a higher likelihood of progression or death. In fact, 6/7 pre-purge marrow-positive patients succumbed to NB and died. Our interpretation is that, despite the ability of 3F8 to purge marrows to RT-PCR negativity, the quality of marrow remission at the time of BM harvest and not the quality of purging correlated with clinical outcome. This is in agreement with the recent report by Seeger et al that increasing tumor cell content at BM collection had an adverse effect on event-free survival.⁴⁰ In the current practice of leukapheresis, NB tumor cell content was found to be 10-100 fold lower that that in the bone marrow.⁴¹ Thus, even though the molecular monitoring of GD2 synthase transcript was carried out on BM harvests, our conclusions should be applicable to PBSC collections.

This retrospective study with a small sample size seems to suggest that even though ex vivo purging might be safe and efficacious as defined by tumor cell depletion, it may not be necessary since the absence of tumor cells in the purged marrow had no impact on patient survival. In all likelihood, the ongoing multi-institutional randomized trial on purged versus un-purged peripheral blood stem cell transplant following dose intensive induction therapy for high risk neuroblastoma (Protocol A3973) initiated by the Children Oncology Group will potentially be able to resolve the question if tumor cells depletion after ex vivo purging has any clinical relevance.

TABLE 1
Quantitation of GD2-positive cells in bone
marrows by immunocytology in 10 patients with
relapsed/refractory stage 4 NB

| | | % GD2 + cells |
|---|---|---|
| Patient | Pre-purged | Post-purged |
| 1 | neg | neg |
| 2 | nd | nd |
| 3 | 0.255 | 0.0035 |
| 4 | nd | nd |
| 5 | neg | neg |
| 6 | 0.082 | neg |
| 7 | 0.005 | neg |
| 8 | 0.010 | neg |
| 9 | 0.002 | neg |
| 10 | 0.002 | neg |

Negative was defined as ≦0.001% GD2 + cells.
nd = not done

TABLE 2
In vitro hematopoietic stem cell recovery following ex vivo
3F8 purging

| | | % | | | |
|---|---|---|---|---|---|
| Protocol | N | TNC Recovery | % CD34 | % CFU-GM | % BFU-E |
| Pilot | 10 | 81.0 ± 20.8 | nd | 102.0 ± 38.0 | 81.0 ± 36.4 |
| | | (10)* | | (7) | (6) |
| N7 | 31 | 87.6 ± 2.3 | 87.43 ± 5.14 | 104.7 ± 11.3 | 85.5 ± 12.6 |
| | | (31)* | (27) | (25) | (24) |

*number of evaluable samples.

TABLE 3
Measurement of GD2 synthase mRNA by real-time
quantitative RT-PCR of pre-purged and post-purged
bone marrows of N7 patients

GD2 Synthase/GAPDH

| No. of patients | Pre-purged | Post-purged |
|---|---|---|
| 24 | ≦5^{a} | ≦5 |
| 6 | 6.1-80.1 | ≦5 |
| 1 | 39.8 | 36.9 |

^{a}GD2 Synthase ≦5 was defined as negative.

### REFERENCES

1. Matthay K K, Villablanca J G, Seeger R C, Stram D O, Harris R E, Ramsay N K, et al. Treatment of high-risk neuroblastoma with intensive chemotherapy, radiotherapy, autologous bone marrow transplantation, and 13-cis-retinoic acid. N Engl J Med 1999;341:1165-73.

2. Brenner M K, Rill D R, Moen R C. Gene marking to trace origin of relapse after autologous transplants of relapse after autologous bone marrow transplantation. Lancet 1993;341:85-86.

3. Rill D R, Santana V M, Roberts W M, Nilson T, Bowman L C, Krance R A, et al. Direct demonstration that autologous bone marrow transplantation for solid tumors can return a multiplicity of tumorigenic cells. Blood 1994; 84:380-3.

4. Yeager A M, Kaizer H, Santos G W, Saral R, Colvin O M, Stuart R K, et al. Autologous bone marrow transplantation in patients with acute nonlymphocytic leukemia, using ex vivo marrow treatment with 4-hydroperoxycyclophosphamide. N Engl J Med 1986;315:141-47.

5. Beaujean F, Hartmann O, Benhamou E, Lemerle J, Duedari N. Hemopoietic reconstitution after repeated autologous transplantation with mafosfamide-purged marrow. Bone Marrow Transplantation 1989;4:537-41.

6. Kemshead J T, Heath L, Gibson F M, Katz F, Richmond F, Treleaven J, et al. Magnetic microspheres and monoclonal antibodies for the depletion of neuroblastoma cells from bone marrow: experiences, improvements and observations. Br J Cancer 1986;54:771-78.

7. Reynolds C P, Seeger R C, Vo D D, Black A T, Wells J, Ugelstad J. Model system for removing neuroblastoma cells from bone marrow using monoclonal antibodies and magnetic immunobeads. Cancer Res 1986;46:5882-86.

8. Merino M E, Navid F, Christensen B L, Toretsky J A, Hellman L J, Cheung N K V, et al. Immunomagnetic purging of Ewing's sarcoma from blood and bone marrow: Quantitation by real-time polymerase chain reaction. J Clin Oncol 2001;19:3649-59.

9. Ritz J, Sallan S E, Bast Jr R C, Lipton J M, Clavell L A, Feeney M, et al. Autologous bone-marrow transplantion in CALLA-positive acute lymphoblastic leukemia after in-vitro treatment with J5 monoclonal antibody and complement. Lancet 1982;2:60-63.

10. Saarinen U M, Coccia P F, Gerson S L, Pelley R, Cheung N K V. Eradication of neuroblastoma cells in vitro by monoclonal antibody and human complement: method for purging autologous bone marrow. Cancer Res 1985;45:5969-75.

11. Gulati S, Atzpodien J, Lemoli R M, Schimazaki C, Clarkson B. Photoradiation methods for purging autologous bone marrow grafts. Prog Clin Biol Res 1990;333:87-102.

12. Leung W, Chen A R, Klann R C, Moss T J, Davis J M, Noga S J, et al. Frequent detection of tumor cells in hematopoietic grafts in neuroblastoma and Ewing's sarcoma. Bone Marrow Transplantation 1998;22:971-79.

13. Donovan J, Temel J, Zuckerman A, Gribben J, Pang J, Pierson G, et al. CD34 selection as a stem cell purging strategy for neuroblastoma: preclinical and clinical studies. Med Pediatr Oncol 2000;35:677-82.

14. Meck M M, Wierdl M, Wagner L M, Burger R A, Guichard S M, Krull E J, et al. A virus-directed enzyme prodrug therapy approach to purging neuroblastoma cells from hematopoietic cells using adenovirus encoding rabbit carboxylesterase and CPT-11. Cancer Res 2001;61:5083-89.

15. Schulz G, Cheresh D A, Varki N M, Yu A, Staffileno L K, Reisfeld R A. Detection of ganglioside GD2 in tumor tissues and sera of neuroblastoma patients. Cancer Res 1984;44:5914-20.

16. Kramer K, Gerald W, Kushner B H, Larson S M, Hameed M, Cheung N K V. Disialoganglioside GD2 loss following monoclonal antibody therapy is rare in neuroblastoma. Clin Cancer Res 1998;4:2135-39.

17. Cheung N K, Lazarus H, Miraldi F D, Abramowsky C R, Kallick S, Saarinen U M, et al. Ganglioside GD2 specific monoclonal antibody 3F8- a phase I study in patients with neuroblastoma and malignant melanoma. J Clin Oncol 1987;5:1430-40.

18. Handgretinger R, Baader P, Dopfer R, Klingebiel T. Reuland P. Treuner J, et al. A phase I study of neuroblastoma with the anti-ganglioside GD2 antibody 14.G2a. Cancer Immunol. Immunother. 1992;35:199-204.

19. Cheung N K V, Walter E I, Smith-Mensah W H, Ratnoff W D, Tykocinski M L, Medof M E. Decay-accelerating factor protects human tumor cells from complement-mediated cytotoxicity in vitro. J Clin Invest 1988;81:1122-28.

20. Chen S, Caragine T. Cheung N K, Tomlinson S. Surface antigen expression and complement susceptibility of differentiated neuroblastoma clones. Am J Pathol 2000;156:1085-91.

21. Honsik C J, Jung G, Reisfeld R A. Lymphokine-activated killer cells targeted by monoclonal antibodies to the disialogangliosides GD2 and GD3 specifically lyse human tumor cells of neuroectodermal origin. Proc. Natl Acad Sci, USA 1986;83:7893-97.

22. Barker E, Reisfeld R A. A mechanism for neutrophil-mediated lysis of human neuroblastoma cells. Cancer Res 1993;53:362-67.

23. Kushner B H, Cheung N K. GM-CSF enhances 3F8 monoclonal antibody-dependent cellular cytotoxicity against human melanoma and neuroblastoma. Blood 1989;73(7):1936-41.

24. Munn D H, Cheung N K. Interleukin-2 enhancement of monoclonal antibody-mediated cellular cytotoxicity (ADCC) against human melanoma. Cancer Res 1987;47:6600-05.

25. Munn D H, Cheung N K. Phagocytosis of tumor cells by human monocytes cultured in recombinant macrophage colony-stimulating factor. J. Exp. Med. 1990;172:231-37.

26. Furukawa K, Soejima H. Niikawa N, Shiku H. Genomic organization and chromosomal assignment of the human β1,4-N-acetylgalactosaminyltransferase gene. J Biol Chem 1996;271:20836-44.

27. Cheung I Y, Cheung N K V. Quantitation of marrow disease in neuroblastoma by real-time reverse transcription-PCR. Clin Cancer Res 2001;7:1698-705.

28. LoPiccolo M S, Cheung N K V, Cheung I Y. GD2 Synthase: a new molecular marker for detecting neuroblastoma. Cancer 2001;92:924-31.

29. Hoon D S B, Kuo C T, Wen S, Wang H, Metelitsa L, Reynolds C P, et al. Ganglioside GM2/GD2 synthetase mRNA is a marker for detection of infrequent neuroblastoma cells in bone marrow. Am J Pathol 2001;159(2):493-500.

30. Cheung N K, Kushner B H, LaQuaglia M, Kramer K, Gollamudi S, Heller G, et al. N7: A novel multi-modality therapy of high risk neuroblastoma (NB) in children diagnosed over 1 year of age. Med Pediatr Oncol 2001;36:227-30.

31. Stiff P J, Koester A R, Weidner M K, Dvorak K, Fisher R I. Autologous bone marrow transplantation using unfractionated cells cryopreserved in dimethylsulfoxide and hydroxyethyl starch without controlled-rate freezing. Blood 1987;70(4):974-8.

32. Cheung N K, Heller G, Kushner B H, Liu C, Cheung I Y. Detection of metastatic neuroblastoma in bone marrow: when is routine marrow histology insensitive? J Clin Oncol 1997;15:2807-17.

33. Kushner B H, Gulati S C, Cheung N K V. Effective purging of neuroblastoma from bone marrow (BM) using IgG3 monoclonal antibody (MoAb) that mediate human complement dependent as well as cell-mediated cytotoxicities. Blood 1988;72(Suppl 1):394a.

34. Tchirkov A, Kanold J, Giollant M, Halle-Haus P, Berger M, Rapatel C, et al. Molecular Monitoring of Tumor Cell Contamination in Leukapheresis Products From Stage IV Neuroblastoma Patients Before and After Positive CD34 Selection. Med Pediatr Oncol 1998;30:228-32.

35. Kushner B H, O'Reilly R J, Mandell L R, Gulati S C, LaQuaglia M, Cheung N K. Myeloablative combination chemotherapy without total body irradiation for neuroblastoma. J Clin Oncol 1991;9:274-79.

36. Moss T J, Sanders D G, Lasky L C, Bostrom B. Contamination of peripheral blood stem cell harvests by circulating neuroblastoma cells. Blood 1990;76:1879-83.

37. Miyajima Y, Horibe K, Fukuda M, Matsumoto K, Numata S, Mori H, et al. Sequential detection of tumor cells in the peripheral blood and bone marrow of patients with stage IV neuroblastoma by the reverse transcription polymerase chain reaction for tyrosine hydroxylase mRNA. Cancer 1996;77:1214-19.

38. Lode H N, Handgretinger R. Schuermann U, Seitz G, Klingebiel T, Niethammer D, et al. Detection of neuroblastoma cells in CD34+ selected peripheral peripheral stem cells using a combination of tyrosine hydroxylase nested RT-PCR and anti-ganglioside GD2 immunocytochemistry. Eur J Cancer 1997;33:2024-30.

39. Saarinen U, Wikstrom S, Makipernaa A, Lanning M, Perkkio M, Hovi L, et al. In vivo purging of bone marrow in children with poor-risk neuroblastoma for marrow collection and autologous bone marrow transplantation. J Clin Oncol 1996;14:2791-802.

40. Seeger R C, Reynolds C P, Gallego R, Stram D O, Gerbing R B, Matthay K K. Quantitative tumor cell content of bone marrow and blood as a predictor of outcome in stage IV neuroblastoma: a Children's Cancer Group study. J Clin Oncol 2000;18:4067-76.

41. Faulkner L B, Garaventa A, Paoli A, Tintori V, Tamburini A, Lacitignola L, et al. In vivo cytoreduction studies and cell sorting—enhanced tumor-cell detection in high-risk neuroblastoma patients: implications for leukapheresis strategies. J Clin Oncol 2000;18(22):3829-36.

Fifth Series of Experiments

Molecular Detection of Occult Neuroblastoma

In the Preliminary Results section, new findings were included, further demonstrating the clinical utility of molecular marker GD2 synthase by quantitative RT-PCR in evaluating purging efficacy (Cancer 2002, in press, ref. 47), and in evaluating one phase of adjuvant therapy in the N7 protocol (submitted for publication, ref. 48).

1. Response to “Unknown Effect of Storage Conditions an mRNA”

To date, 2000 cyropreserved bone marrows and blood samples have been processed. The quality of mRNA is determined by (a) PCR of the housekeeping gene β2 microglobulin and the detection of the specific PCR product by agarose gel electrophoresis, (b) quantitation of another housekeeping gene glyceraldehyde-3-phosphate dehydrogenase (GAPDH) by real-time RT-PCR. Thus far, good quality mRNA in 97% of the samples have been attained, indicating storage of frozen specimens did not have appreciable deleterious effect. In separate experiments mRNAs were extracted from fresh and cryopreserved aliquots, and their integrity was comparable under these conditions.

2. Response to “Proposed Use of a Chemiluminescent Technique for One Marker and a Quantitative Real-Time RT-PCR for the Other One”

In our method development, chemiluminescent technique precedes real-time RT-PCR for both markers GAGE1 and GD2 synthase. The detection of one tumor cell in the presence of one million normal mononuclear cells in GAGE1⁴⁰ and GD2 synthase has been demonstrated by RT-PCR and chemiluminescence.⁴⁵ Quantitative RT-PCR gives added information because of its quantitative aspect. The technique for GD2 synthase has been validated⁴⁶ and development of a real-time assay for GAGE1 is expected to be completed. Having two independent detection modalities, such as chemiluminescence versus real-time, may also be potentially more informative.

3. Response to “Inclusion of Microarray Studies in Aim 2B”

The microarray studies have been removed from this proposal at the suggestion of the reviewers. The two hypotheses in this grant have been retained and clarified, i.e. immunotherapy is as effective as marrow/stem cell transplant in the adjuvant setting when patients are in apparent clinical remission (aim 1), and MRD at the end of induction, and at 2 years from diagnosis have the strongest prognostic impact on patient survival (aim 2).

Our aim 1 is an efficacy (response) study, examining the change in occult tumor cells (MRD) as measured by these two molecular markers in marrows sampled before and after treatment in both immunotherapy and marrow/stem cell transplant. In this revised grant, it was detailed how MRD response will be correlated with the level of pre-treatment tumor content, as well as clinical/biologic markers known to impact on patient outcome. For the immunotherapy group, host factors known to affect tumor killing by antibody 3F8 will also be tested for their impact on MRD response.

Our aim 2 uses survival as the endpoint. Here our cohort is a fairly uniform group of patients who were (1) all newly diagnosed stage 4 patients (2) ranging from 1-17 years of age (3) treated at Sloan-Kettering by consecutive neuroblastoma protocols, N5, N6, N7, and N8. The marrows of these patients is planned at defined time points, namely at diagnosis, end of induction (right before transplant or 3F8 adjuvant therapy), 12, 18, and 24 months from diagnosis. The presence and magnitude of MRD at these time points will be analyzed for their prognostic importance in survival analysis. A multivarate model that includes known prognostic factors (serum ferritin, serum LDH, tumor histology, MYCN amplification, histology, chromosome 1p36 LOH, 17q gain and the presence of anti-idiotype network) will also be used. Microarray data will not be included. This should provide an assessment of the critical timing of MRD measurement in predicting patient outcome.

4. Response to “Major Concern Involves the Weak Statistical Considerations of the Proposal”

Statistical analysis for both aims has been completely rewritten.

Aim I

1. In order to evaluate efficacy of adjuvant therapy BMT/SCT, two statistical methods will be used since there are two PCR measurements, GAGE1 result being a binary variable, whereas GD2 synthase result is a continuous variable. The change rates from a pre-treatment positive sample turning negative as detected by GAGE1 can be estimated by using a binomial assumption on the treatment effect. A paired T-test can be used for GD2 synthase findings. The same method will be used for evaluating the efficacy of 3F8 immunotherapy.

2. Comparison in efficacy between the two adjuvant therapies will be accomplished by using Fisher's exact test for GAGE1 results.

3. With the GD2 synthase data, the functional relationship between baseline level y1 and the percentage change d (defined as d=(y2−y1)/y1, where y2 is the GD2 synthase level after treatment) will be found. Comparison in efficacy between the two adjuvant therapies will be accomplished by comparing the underlying parameters.

4. To test if MRD response is correlated with clinical or biologic markers, a generalized linear model, based on the works by Liang and Zeger (ref. 53), and Fitzmaurice and Laird (ref. 54) on longitudinal data analysis will be used.

Aim II

1. Univariate survival analysis will be performed in order to determine variables that can predict progression-free and overall survival. The multivariate and time dependent Cox regression model will be used to evaluate whether MRD can predict survival, both progression-free and overall, after adjusting for the covariate effects.

2. Generalized linear model will be used to find the correlation between GAGE1 and GD2 synthase and clinical or biologic variables. Longitudinal data analysis techniques will be used by taking into account there are multiple measurements of GAGE1 and GD2 synthase at different time points.

3. In order to find a functional relationship between PCR levels with time, the PCR level will be modeled as a nonlinear function of time plus an unobservable error. Different nonlinear models will be used to fit the observed data.

4. Possible non-ignorable missing data due to relapse or death at time points further away from diagnosis by using statistical analysis based on recent research works by Lipsitz et al. (ref 55), and Troxel et al. (ref 56) will be taken into account.

5. If needed, survival analysis taking into account missing covariate data in the Cox regression model will be performed.

Specific Aims:

Minimal residual disease (MRD) is one of the final hurdles to cancer cure. This application proposes to utilize two molecular markers, namely GD2 synthase mRNA (β1,4-N-acetylgalactosaminyltransferase) by real-time quantitative RT-PCR, and cancer/testis antigen GAGE1 by RT-PCR and chemiluminescent detection, to measure MRD response (aim 1) and evaluate their prognostic impact (aim 2) in the bone marrows of a large cohort of patients with high risk neuroblastoma. In earlier studies with small cohorts of patients with neuroblastoma and melanoma who were in apparent clinical remission, the presence of GD2 synthase mRNA and cancer/testis antigen GAGE1 in blood and marrow was found to be prognostic of patient survival. Furthermore, by real-time quantitative RT-PCR, the level of GD2 synthase mRNA correlated well with the number of GD2-positive tumor cells as measured by immunocytology in marrow samples from patients with neuroblastoma, a high expressor of GD2.

Specific Aim 1:

To evaluate and compare the efficacy of two important adjuvant therapies in neuroblastoma, marrow/stem cell transplantation (patient N=139) and anti-GD2 monoclonal antibody 3F8 immunotherapy (patient N=269) from a single institution. Cryopreserved marrows before and after these adjuvant therapies from a large consecutive patient cohort diagnosed with high-risk neuroblastoma spanning the past 14 years will be evaluated for MRD response. Efficacy will be determined by the percentage of patients whose marrows which are positive by RT-PCR before treatment become negative following treatment, as well as their magnitude of response. MRD response will also be correlated with the level of pre-treatment tumor content. It is hypothesized that antibody treatment is as effective as marrow/stem cell transplant. It will be tested to see if clinical/biologic markers at diagnosis (ploidy, MYCN, 1p36LOH, 17q gain, serum LDH and serum ferritin), known to impact on patient outcome, will correlate with MRD response of either adjuvant therapy. Additionally, host factors (ANC, ALC, serum complement) that mediate tumor killing by 3F8 will be correlated with efficacy of immunotherapy.

Specific Aim 2:

To test the prognostic significance of MRD on progression-free survival and overall survival in a cohort of unselected consecutive and newly diagnosed stage 4 neuroblastoma patients ranging from 1-17 years of age (patient N=130) who were treated on consecutive MSKCC protocols N5, N6, N7, N8 evolving over the last 14 years. MRD from their marrows as detected by RT-PCR of GD2 synthase mRNA and GAGE1 will be evaluated at diagnosis, end of induction (right before transplant or 3F8 treatment), at 12, 18, and 24 months from diagnosis. Based on our preliminary findings, it is hypothesized that MRD at the end of induction and at 2 years from diagnosis will have the strongest prognostic impact on patient survival. It will also be tested to see if MRD has an independent prognostic impact in predicting patient outcome, when known clinical, biologic, and treatment variables (serum ferritin, serum LDH, tumor histology (Shimada classification), MYCN amplification, chromosome 1p36LOH, 17q gain and the presence of anti-idiotype network) are included in the multivariate model. It is hypothesized that MRD, being the final common pathway of inadequately treated or resistant cancer, will be a dominant determinant of outcome, as adverse tumor biology continues to be neutralized by increasingly effective treatment strategies.

Since 1987 all the serial marrow and tumor samples from patients treated at MSKCC with marrow/stem cell transplant and 3F8 were cryopreserved. Data on the biology of these tumors and the clinical information on these patient cohorts are comprehensive. Extensive genome-wide allelic analysis on most of these tumor samples is available. For patients treated with 3F8 containing regimens, information on their anti-idiotype network is also available. If these molecular markers of MRD can independently predict patient outcome, they may provide an objective endpoint for stopping treatment, and serve as sensitive surrogates in our evaluation of adjuvant therapies.

Background and Significance:

A major hurdle to cancer cure is MRD, especially when the cancer has become metastatic, which ultimately can lead to death. MRD are residual tumor cells persisting in bone marrow (BM), peripheral blood (PB), lymph nodes or cerebral spinal fluid (CSF), despite apparent clinical remission with no obvious masses capable of tumor shedding. Monitoring MRD should be an essential part of cancer management. Detection of MRD can help develop accurate prognostic profiles for patient at diagnosis and during treatment, such that more appropriate therapy can be delivered to the individual patients. Examining BM and PB with successive cycles of chemotherapy helps document the “kinetics” of response of metastatic disease.¹ As a surrogate endpoint, measurement of MRD reports the efficacy of biologic/adjuvant therapies, without the lengthy observation period pending clinical relapse or death. Accurate quantitation of occult tumor cells can also define the optimal time to obtain autologous stem cells prior to transplantation, since infused tumor cells are clearly responsible for post-transplant relapse,^{2,3} and the risk of tumor contamination of autografts even in chemo-responsive patients is considerable.⁴ Detection of MRD will help evaluate the optimal timing and efficacy of various stem-cell purging techniques.

At Memorial Sloan-Kettering Cancer Center (MSKCC), patients with metastatic neuroblastoma (NB) diagnosed after one year of age were treated on consecutive protocols evolving over the years: N4, N5, N6, N7 and currently N8. While N5 patients were only imaged with monoclonal antibody (MoAb) 3F8, all subsequent regimens used 3F8 treatment as adjuvant immunotherapy/radioimmunotherapy. Overall progression-free survival (PFS) has steadily increased from 25% in N5 to >50% in N7. Thus far, there were no relapses after 3 years of continual remission in any of the 3F8-treatment regimens, leading to the hope that cures may be possible. However, besides NB relapse, treatment-related leukemia (t-AML) and isolated central nervous system (CNS) relapse have emerged as new obstacles to cure. The intensity of topoisomerase-II inhibitors and alkylators during induction chemotherapy likely entails a considerable leukemogenic risk. On the other hand, despite multiple rounds of these intensive therapies, CNS remains a sanctuary site for NB cells, since the blood-brain barrier renders both systemic chemotherapy and immunotherapy ineffective. It is reasoned that reducing chemotherapy (especially if complete remission can be achieved and identified earlier) can lower the incidence of t-AML, and early detection and prophylaxis of CNS disease may benefit those patients at risk. These objectives can be achieved only by having an accurate monitoring of minimal residual disease (MRD) in BM, PB and CSF.

Optimal MRD assays must achieve the following criteria. First, they must have superior sensitivity and specificity. Second, they should correlate with clinical outcome, ideally with independent significance in multivariate analyses, where standard clinical and biologic factors are fully taken into account. The clinical significance of MRD needs to factor in tumor biology. For example, stage 4s NB patients are well known to have marrow disease, and yet they predictably have favorable outcome. The combination of MRD measurement and genetic profiling of tumor biology in the context of relatively uniform treatment methods will clarify if MRD is indeed clinically relevant for all patients, or only for specific subgroups. One clinical endpoint of great value in the study of MRD is patient survival, both progression-free and overall survival. Although MRD measurement is tested at fixed time points from diagnosis to ensure statistical validity in these proposed studies, once the principle is proven, knowing the level of MRD after each phase of treatment and during off-therapy followup has obvious clinical utility.

In solid tumors including neuroblastoma, BM is a frequent site of metastatic disease, and is often the first and only site of relapse. Large retrospective studies have clearly demonstrated the importance of marrow remission in ensuring long-term survival.⁵ Evidence points to the presence of MRD in the marrow of breast cancer patients at the time of primary surgery being a strong predictor of relapse.⁶ Presence of occult tumor cells in marrow aspirates obtained around the time of ABMT was highly predictive of relapse.⁷ since tumor cells could mobilize from bone marrow to blood, and seed other organ sites.^{8,9} The quantity of marrow disease may also be important.¹⁰ In childhood acute lymphoblastic leukemia, MRD detected by polymerase chain reaction (PCR) at the end of induction therapy could be a factor in predicting subsequent relapse.¹¹ Occult tumor cells are likely to be heterogeneous with respect to genetic characteristics, metastatic potential, and chemoresistance. Histological, immunological, and molecular methods have been used to detect MRD. Conventional cytology has a detection sensitivity of 1/10³, immunocytology has a sensitivity of 1/10⁵ to 1/10⁶, whereas PCR is in the range of 1/10⁵ to 1/10⁷. Specificity of the assay is often influenced by the source of tissue analyzed, and the quality of antibodies used. Cross-reactivity with blood or marrow cells poses severe limitations. Clearly, immunocytology has one strong advantage, namely the enumeration of the exact number of tumor cells in any sample. In addition, it can exploit a mixture of monoclonal antibodies of different specificities, and allow the morphology of individual cells to be verified. However, this assay can be labor intensive, and the theoretical gain of antibody panels has so far been limited by the cumulative background staining and cross reactivity with hematopoietic cells.

On the other hand, molecular-based MRD assays can be hampered by inherent pitfalls of DNA amplification. “Tumor-specific” genes are occasionally transcribed even in normal tissues. For example, neuroendocrine protein gene product, PGP 9.5, a potential marker of neuroblastoma, was shown to be detectable in some normal BM and PBL. ^{12,13} There is also the problem of illegitimate transcription, i.e. the transcription of any genes in any cell types. A good example is prostate-specific antigen transcript, which is detected in both normal and malignant breast/endometrial tissues.¹⁴ In addition, pseudogenes lack intronic sequences, resulting in PCR products indistinguishable form those generated from the mRNA. Expression of cytokeratin pseudogenes in hematopoietic cells in breast cancer was frequently reported.¹⁵ Non-hematopoietic markers can also be introduced into the circulation after invasive procedures, leading to false positive PCR results. Lastly, DNA cross-contamination is not uncommon in a restricted laboratory environment. False negative results in molecular assays can be due to the presence of inhibitors, tumor cell heterogeneity, technical errors, sampling problem, as well as down-regulation of the target gene by therapy. PSA mRNA expression was decreased after anti-androgen therapy.¹⁶ This may also hold true for tyrosine hydroxylase (TH), the rate-limiting enzyme in catecholamine synthesis. Because of its feedback regulation, its gene expression in neuroblastoma is inversely correlated with that of the noradrenaline transporter, which carries meta-iodo-benzylguanidine (MIBG) into tumor cells.¹⁷ The abundance of this transporter in advanced-stage neuroblastomas is evident by tumor avidity for MIBG. Here, tyrosine hydroxylase expression could be down-regulated. Because of tumor heterogeneity, MRD surveillance is likely to require the use of multiple markers. The most serious pitfall with molecular techniques is the lack of correlation between mRNA or DNA copy number and the actual tumor cell number, the gold standard of MRD, thus potentially limiting their clinical relevance.

Recently, several research groups reported the impact of MRD in neuroblastoma on clinical outcome. In a prospective multicenter study in UK, the presence of tyrosine hydroxylase mRNA in the blood of stage 4 NB patients at diagnosis and when they were clinically free of disease was found to be an independent prognostic factor for event-free and overall survival.¹⁸ Detection of TH mRNA in the blood of patients on treatment was not predictive of outcome. In contrast, Fukuda et al found that persistent TH mRNA in the BM more than 4 months after the start of chemotherapy among stage 4 NB patients was associated with poor prognosis.¹⁹ In a much larger series of stage 4 NB patients in the CCG study 3891, BM and PB were tested for tumor cells by immunocytology at diagnosis, week 4, week 12, BM harvest, and at the end of induction chemotherapy. Positive blood immunocytology at diagnosis was associated with decreased EFS in bivariate Cox analyses. In addition, EFS was also inferior if BM was positive for NB at diagnosis (p=0.04), at 12 weeks (3 cycles of chemotherapy) (p=0.006), and at bone marrow harvest (p<0.001).²⁰ Indeed, if MRD at diagnosis proves to have such a strong predictive power on clinical outcome independent of known clinical, biologic, and treatment variables, a stronger emphasis on better therapeutic strategies for this metastatic disease must be placed.

Preliminary Studies:

The neuroblastoma program at Memorial Sloan-Kettering Cancer Center is devoted to the research, education and treatment of NB. ongoing translational research from bench to the bedside is evidenced by over 20 IRB-approved protocols for neuroblastoma in the past decade. These clinical trials have included autologous bone marrow transplant,^{21,22} chemotherapy induction protocols,²³ phase I and II antibody studies.^{24,25} In the past decade, >150 new patients with NB, most of them over one year of age at diagnosis, were treated at MSKCC. Over 240 patients have undergone MoAb 3F8 therapy, which is now routinely done in the outpatient clinic. Accurate monitoring of occult tumor cells is an essential part of cancer management. Both immunological and molecular assays which have superior specificity and sensitivity to monitor MRD have been developed and validated.

GD2 is Homogeneously Expressed in NB of All Stages.

GD2 density on neuroblastoma cells is high (5-10×10⁶ molecules per cell)²⁶ and this glycosphingolipid antigen is rarely lost after anti-GD2 therapy.²⁷ In immunocytology freshly collected heparinized bone marrow pooled from four aspiration sites was separated by Ficoll centrifugation and mononucleated cells were incubated with a panel of anti-GD2 monoclonal antibodies, followed by a reaction with a fluoresceinated anti-mouse IgG antibody. GD2-positive tumor cells were examined and enumerated using a fluorescence microscope and quantitation was expressed as percentage of GD2-positive cells/total number of mononuclear cells in each counting chamber.²⁸ Our study of 145 stage 4 NB patients who underwent 840 marrow examinations demonstrated that immunocytology had superior sensitivity compared to histological examinations of multiple BM biopsies or aspirates (FIGS. 14 and 15). Because of the specificity of anti-GD2 monoclonal antibodies for the oligosaccharide moiety of GD2, little background staining was observed. Immunocytology using specific antibodies against GD2 has been successful in detecting and quantifying tumor cells in the BM.^{28,29} Its limit of detection is 1 tumor cell in 10⁵ nucleated cells,^{10,30} far more sensitive than conventional histology.²⁸

Molecular monitoring of residual tumor cells by RT-PCR utilizes cryopreserved mononuclear cells, and experiments can be repeated multiple times and for multiple markers as they are being developed. This is in contrast to immunocytology where only freshly collected samples are used because archived samples are unable to give reproducible results. In our laboratory, cancer/testis antigens (CTA) have been explored to evaluate their potentials as markers for MRD. Included in our study were MAGE,³¹ GAGE1,³² BAGE,³³ NY-ESO-1³⁴ and SSX.³⁵ CTAs are a growing class of genes that encode tumor-associated proteins recognized by autologous cytolytic T-lymphocytes.³⁶ These antigens have also been identified through their ability to elicit host autologous antibody responses³⁷ and their potentials as targets in vaccine immunotherapy. Common characteristics of CTAs are their expression in human cancers of diverse lineages, their existence in multi-gene families, and their localization on the X chromosome, with restricted expression in normal tissues except testis (MAGE genes are also expressed in placenta and fetal tissues³⁸). In our exploration of CTA as molecular marker, assays were based on RT-PCR, electrophoretic separation of biotinylated PCR products, and a chemiluminescent detection technique, which was at least 500-fold more sensitive than ethidium bromide visualization.³⁹ (FIG. 16)

The Advantage of Multiple RT-PCR Markers

The use of multiple molecular markers to overcome antigen heterogeneity was explored. Sixty-seven NB frozen tumors (12 stage 1, 13 stage 2, 12 stage 3, 12 stage 4S, and 18 stage 4) were assayed for CTA expression.³⁹⁻⁴¹ (Table 1)

TABLE 1
Expression of multiple cancer/testis antigens in
neuroblastoma tumors according to stage

| | Number of positive tumors | | | | |
|---|---|---|---|---|---|
| | Stage | | | | |
| | 1 | 2 | 3 | 4S | 4 |
| Total tumors | 12 | 13 | 12 | 12 | 18 |
| GAGE1 | 8 | 13 | 9 | 7 | 18 |
| MAGE-1 | 6 | 8 | 7 | 6 | 7 |
| MAGE-2 | 8 | 8 | 8 | 7 | 10 |
| MAGE-3 | 11 | 9 | 10 | 8 | 13 |
| MAGE-4 | 8 | 7 | 7 | 4 | 15 |
| BAGE | 10 | 7 | 6 | 7 | 16 |
| SSX-2 | 4 | 5 | 7 | 4 | 13 |
| SSX-4 | 2 | 4 | 2 | 2 | 12 |
| NY-ESO-1 | 4 | 6 | 5 | 3 | 4 |

82% of tumors had positive expression for GAGE1, 51% for MAGE-1, 61% for MAGE-2, 76% for MAGE-3, 61% for MAGE-4, 69% for BAGE, 49% for SSX-2, and 33% for SSX-4 and 33% for NY-ES-O1. SSX-2 and SSX-4 detection was correlated with clinical stage, with higher frequency of expression detectable among stage 4 tumors. As for the rest of the CTAs, there was no statistically significant correlation between expression and stage. Interestingly, among stage 4 tumors, 100% had detectable GAGE1 expression, suggesting this marker may potentially be useful for poor-risk stage 4 patients.

Sensitivity and Specificity of Marker Gage1

Spiking experiment using serially diluted NB cell line NMB7 in normal marrows cells demonstrated the detection limit of GAGE1 expression (239 bp) was 1/10⁶ (FIG. 17).⁴⁰ Using normal BM and PBL, as well as non-NB remission marrows (n=48), GAGE1 specificity was confirmed.⁴²

By means of remote-controlled hydraulic micro-manipulators (Narishige Co., Japan), the sensitivity of GAGE1 in detecting tumor cells was further tested. A SINGLE tumor cell of various histological types (including 8 NB, 2 PNET, 2 medulloblastoma, 1 breast CA, 1 osteogenic sarcoma, 1 rhabdomyosarcoma, and 1 melanoma) was spiked in a million normal peripheral mononuclear cells. GAGE1 expression was detectable at the single cell level of all the tumor types studied.

The utility of GAGE1 as a maker of MRD was validated in an ongoing collaborative study with the melanoma service at MSKCC.⁴² One -hundred thirty-two patients with malignant melanoma (21 clinical stage II, 74 stage III, and 38 stage IV) had a single marrow and/or blood sample drawn immediately prior to definitive surgery. These samples were coded such that investigators had no prior knowledge of the patients' clinical status. GAGE1 detection was correlated with adverse patient survival, measured from the sampling time (p=0.01). In a multivariate model, only GAGE1 positivity in blood and/or marrow and clinical stage were significant prognostic variables.

In a follow-up study,⁴³ the prognostic significance of GAGE1 and tyrosinase transcript, a sensitive and specific marker of melanoma, using a single sample of BM and/or PB in 125 AJCC stage III melanoma patients who were rendered surgically free of disease was assessed. In a multivariate analysis GAGE1 positivity, when compared to other variables which included tyrosinase, number of positive lymph nodes, immunotherapy, and location of primary site, was found to be an important independent predictor of PFS (p<0.001) and overall survival (p<0.001) (Table 2).

TABLE 2
Prognostic factors: multivariate analysis of 125 AJCC
stage III melanoma patients
(Coit, Ghossein, Cheung)

| | | | |
|---|---|---|---|
| Factor | RR | 95% CI | P-value |

Model I. Relapse-Free Survival

| | | | |
|---|---|---|---|
| Tyrosinase-ANY (pos vs neg) | 2.63 | (1.29, 5.40) | 0.008 |
| GAGE-ANY (pos vs neg) | 5.22 | (2.46, 11.05) | <0.001 |
| #Positive nodes (1, 2 vs >3) | 2.55 | (1.31, 4.97) | 0.006 |
| Immunotherapy (yes vs no) | 0.52 | (0.28, 0.99) | <0.05 |
| Location of Primary | | | |
| (Extremity vs Trunk) | 2.21 | (1.09, 4.50) | 0.03 |
| (Unknown vs Trunk) | 0.72 | (0.23, 2.23) | 0.57 |

Model II. Overall Survival

| | | | |
|---|---|---|---|
| Tyrosinase-ANY (pos vs neg) | 2.67 | (1.11, 6.44) | <0.03 |
| GAGE-ANY (pos vs neg) | 5.94 | (2.53, 13.94) | <0.001 |
| #Positive nodes (1, 2 vs >3) | 2.56 | (1.15, 5.70) | <0.03 |
| Immunotherapy (yes vs no) | 0.57 | (0.26, 1.26) | 0.17 |
| Location of Primary | | | |
| (Extremity vs Trunk) | 1.81 | (0.80, 4.12) | 0.16 |
| (Unknown vs Trunk) | 0.39 | (0.08, 1.88) | 0.24 |

ANY = blood or bone marrow

It was then tested whether GAGE1 could be a marker of MRD for advanced NB diagnosed at >1 year of age initially treated with protocol N6 (n=24) and N7 (n=38) at MSKCC.⁴⁴ Their BM cells at 12, 18, and 24 months (median time after diagnosis) were evaluated for the presence of GAGE1. GAGE1 positivity at 12 months (25%), when patients were still on treatment, did not predict PFS and overall survival from the time of sampling. Positivity at 18 months (29%) was associated with poorer PFS and survival (but p>0.05). By 24 months, the presence of GAGE1 (26%) was a strong predictor of survival outcome (p<0.001) (FIGS. 18 and 19). When only remission marrows at 24 months were analyzed, PFS was 4.7-fold lower among GAGE1-positive patients. It is concluded that GAGE1 detection by RT-PCR in the marrow has potential clinical utility, in particular for MRD study, to allow earlier identification of patients at risk, such that appropriate intervention can be initiated before clinical relapse. GAGEI may also serve as a surrogate endpoint for MoAb adjuvant therapy.

In an earlier study,⁴⁰ paired BM and PB samples from 18 patients with metastatic neuroblastoma were tested for GAGE1 and tyrosine hydroxylase (TH) mRNA expression. TH mRNA is a sensitive and specific marker of NB, despite its down-regulation in some cells. Concordance in paired samples was 61% for TH mRNA and 89% for GAGE1. Because GAGE1 expression correlated strongly with other evidence of disease (p=0.07 for BM, and p=0.02 for PB) as measured by marrow histology, immunocytology, bone scan, MIBG scan, CT/MRI, and urine VMA/HVA, it was unlikely that this superior sensitivity over TH was due to false positivity. Thirteen patients were in continual remission off therapy and their GAGE1 expression (12 BM and 9 PB) was undetectable at follow-up. When compared to molecular detection of TH mRNA, a marker frequently used for neuroblastoma, GAGE1 offered added sensitivity in detecting neuroblastoma in both BM and PB.

Another potential molecular marker of MRD, the transcript of GD2 synthase,⁴⁵ was recently explored, since GD2 is abundantly expressed in NB and its synthesis is dependent on a key enzyme β1,4-N-acetylgalactosaminyltransferase (GD2 synthase). By means of RT-PCR and the sensitive chemiluminescent detection, GD2 synthase expression was detectable in all NB tissues of every stage (n=77). More importantly, 30/30 stage 4 tumors were GD2 synthase mRNA positive. This potential molecular marker was found to have high specificity, since none of the normal BM, PBL, and non-NB remission marrows (n=52) tested for the gene expression was detectable by chemiluminescence. GD2 synthase RT-PCR sensitivity was 1/10⁶ cells as illustrated in FIG. 20.

The clinical utility of GD2 synthase for MRD was validated by utilizing a real-time quantitative RT-PCR assay which was recently developed.⁴⁶ The advantages of real-time quantitation are numerous. With a wide linear dynamic range, superior sensitivity and accuracy, it allows good intra-assay and inter-assawy reproducibility. Additional attractions include high throughput capacity, speed, and the elimination of lengthy post-PCR handling steps, preventing potential carryover contamination. A multiplex assay was established by generating two reproducible standard curves, one for GD2 synthase mRNA and one for GAPDH. The cDNA standard (100,000 arbitrary units) was derived from a NB cell line NMB7, and was reverse transcribed in the same manner as the test samples. The standard was serially diluted to obtain a linear dynamic range of >5 logs (FIG. 1). The threshold level, i.e. the upper limit of normal (mean±2 standard error (SE)) of GD2 synthase mRNA was established by using a total of 31 normal BM and peripheral blood samples. Mean GD2 synthase/GAPDH was 0.98 and SE was 1.60. Levels below 5.0 were defined as negative. Sensitivity of this assay was established by spiking NMB7 cells at ratios ranging from 1 to 10,000 tumor cells per million normal marrow mononuclear cells. The quantitative values are tabulated in Table 3. The level of GD2 synthase transcript for a tumor content of 1 in 10⁶ was 9.54±1.74.

TABLE 3
Sensitivity of real-time quantitative RT-PCR of GD2
synthase mRNA

| Ratio of tumor cells | |
|---|---|
| to normal marrow cells | GD2synthase/GAPDH |
| 10⁻² | 581.91 ± 62.41^{a} |
| 10⁻³ | 93.24 ± 4.75 |
| 10⁻⁴ | 24.18 ± 2.06 |
| 10⁻⁵ | 12.18 ± 1.51 |
| 10⁻⁶ | 9.54 ± 1.74 |

^{a}mean ± SE of 2 experiments

155 bone marrows from NB patients were evaluated by means of a multiplex quantitative RT-PCR. Quantitation of GD2 synthase mRNA, normalized to endogenous control glyceraldehyde 3-phosphate dehydrogenase (GAPDH), was compared with measurements derived from immunocytology, where a fluorescence microscope was used to count the number of GD2-positive tumor cells. Marrows positive for both GD2 synthase and immunocytology were included in this correlation analysis. GD2 synthase mRNA levels correlated well with the number of GD2-positive cells (r=0.96) (FIG. 3). This is the first demonstration on the quantitative relationship between a specific mRNA copy numbers and the actual number of tumor cells. Although GAPDH expression could be variable in tumors, its expression level among normal bone marrow cells was constant (forming the bulk of the GAPDH message except when replaced by tumor) and it is consistently so over time in 98% of patients.

The clinical significance of GD2 synthase transcript was then tested among patients diagnosed at >1 year of age with advanced NB whose marrows were sampled 24 months (median time after diagnosis). They were initially treated with protocols N6 and N7 at MSKCC (n=44). Positivity was strongly associated with progression-free (p<0.005) (FIG. 4) and overall survival (p<0.001) (FIG. 5).

In pilot study,⁴⁶ sequential bone marrows from five stage 4 NB patients were studied throughout the course of their treatment and follow-up (Table 4). The expression levels of GD2 synthase correlated closely with the patient's clinical status. Patient 1, 2 and 3 are alive and progression-free. Patient 4 and 5 have succumbed to NB and died.

TABLE 4
Relative quantitation of GD2 synthase mRNA of sequential
samples in the bone marrows of stage 4 neuroblastoma
patients during the course of treatment and follow-up^{a}

| | Patient | | | | |
|---|---|---|---|---|---|
| Timeline | #1 | #2 | #3 | #4 | #5 |
| At diagnosis | 24.7 (0 m)^{b} | 21.0 (0 m) | 107.1 (0 m) | 4098.3 (0 m) | 350.3 (0 m) |
| During treatment | 12.0 (6 m) | 0 (6 m) | 9.9 (6 m) | 37.4 (6 m) | 15.0 (1 m) |
| Clinical remission | 1.8 (12 m) | 0 (12 m) | 0 (12 m) | 0.0 (13 m) | 2.2 (10 m) |
| Follow-up | 4.2 (17 m) | 0 (18 m) | 0 (18 m) | 50.8 (17 m) | 8.5 (13 m) |
| Follow-up | 1.1 (24 m) | 0 (31 m) | 0 (24 m) | 16.2 (24 m) | — |
| Relapse | — | — | — | 25.8 (30 m) | 546.3 (18 m) |
| Relapse | — | — | — | 26.1 (41 m) | — |
| Status | PF^{c} | PF | PF | Dead | Dead |
| | (73 m) | (83 m) | (100 m) | (50 m) | (22 m) |

^{a}Relative quantitation was expressed as GD2 synthase/GAPDH with a detection threshold of 5.0
^{b}Months (m) from diagnosis
^{c}Progression-free

Our studies strongly suggest that combining multiple detection techniques can facilitate the detection of MRD. NB positivity in 259 BM from 99 NB patients was compared using four techniques: histological examination, immunocytology, and molecular detection by GAGE1 and tyrosine hydroxylase mRNA (TH), a sensitive and specific NB marker.³⁹ GAGE1 and immunocytology were found to be more sensitive than histology and TH RT-PCR when marrows were obtained from patients on therapy or off therapy in clinical remission (Table 5). In this study, no correlation with clinical outcome was made.

TABLE 5
Bone marrow positivity by each of 4 detection
methods vs time of sampling

| | NBM Positivity | | | | | |
|---|---|---|---|---|---|---|
| | | | Individual Tests | | | |
| Sample Timing | N | Total+ | GAGE+ | IC+ | HIST+ | TH+ |
| at diagnosis | 13 | 13 | 12 | 13 | 10 | 8 |
| on chemotherapy | 70 | 64 | 54 | 42 | 19 | 24 |
| off chemotherapy | 145 | 106 | 69 | 52 | 17 | 14 |
| at relapse | 31 | 24 | 17 | 20 | 16 | 9 |
| Total | 259 | 207 | 152 | 127 | 62 | 55 |

iTotal+ = positive by ≧1 of 4 tests;
GAGE, TH by RT-PCR;
IC = immunocytology;
tHIST = histology,
+ = positive.

Subsequently, 152 BM from NB patients was tested, and comparisons were made using three independent detection techniques, namely GD2 synthase RT-PCR, immunocytology (IF), and histology.⁴⁵ BM of 24 patients were negative by 3/3 methods; six of them eventually died of the disease. In contrast, among 25 patients who had positive marrows detectable by all three tests, 20 succumbed to the disease (FIG. 21, p<0.01). Patients whose marrows were positive by IF and GD2 synthase RT-PCR also had adverse survival outcome (FIG. 22, p<0.01). Agreement among independent methods would increase the credibility of positive findings, especially from patients who were on treatment, or in apparent clinical remission, when their marrows likely had very few tumor cells. Among patients on chemotherapy who had their BM tested in this study, those who had detectable tumor cells by 2 or 3 tests eventually died of the disease. Relationship between survival status and concomitant marrow positivity was statistically significant (p=0.01). A similar association between clinical outcome and positivity was also observed among patients whose marrows were sampled during their follow-up. It is concluded that combining multiple detection techniques can facilitate the detection of MRD.

Since the last grant submission, the efficacy and safety of ex vivo BM purging using anti-GD2 antibody 3F8 was evaluated by means of quantitative RT-PCR of GD2 synthase mRNA. 3F8 kills NB cells by complement-mediated cytotoxicity and antibody-dependent cell-mediated cytotoxicity. Its purging efficacy in the presence of autologous plasma and leukocytes was tested in (1) a pilot study (1990-1993) in 10 patients with relapsed/refractory stage 4 NB whose BM had <5% tumor content by immunocytology (IF), and (2) 31 stage 4 NB patients who underwent treatment on the N7 protocol (1994-1999) whose remission BM was purged by 3F8 prior to ¹³¹I-3F8 radioimmunotherapy.⁴⁷

GD2 positivity by IF was found in 6/8 patients on the pilot study before purging, 5/6 became negative post-purging. of 31 patients on the N7 protocol, even though pre-purge BM were negative by histology and IF, the more sensitive real-time quantitative RT-PCR detected GD2 synthase mRNA in 7 BM. Six of the 7 marrows became negative after 3F8 purging. Marker positivity prior to purging was statistically significant in predicting overall survival (p=0.04), but not progression-free survival (p=0.1). Purging decreased total nucleated cell count by 12%, CD34+ cells by 12%, BFU-E colony count by 14%; CFU-GM colony count was not damaged. Twenty-four of 31 patients received purged marrow following myeloablative ¹³¹I-3F8 immunotherapy without complications. Median time to engraftment was 19 days for ANC, 49 days for platelet >20K and 102 days for platelet >50K. It is concluded that 3F8 purging of EM was safe and tumor cell depletion quantified by real-time RT-PCR of GD2 synthase demonstrated efficacy.

Since adjuvant therapy is applied at the time of clinical remission, objective surrogate markers are needed to gauge treatment efficacy. Using quantitative RT-PCR of GD2 synthase mRNA, MRD response to anti-GD2 monoclonal antibody 3F8 adjuvant therapy was evaluated, namely one cycle of myeloablative radioimmunotherapy using ¹³¹I-3F8 plus one cycle of unlabeled 3F8 in 45 stage 4 neuroblastoma patients (newly diagnosed/without prior relapse) on the N7 protocol. The prognostic impact of MRD before and after adjuvant therapy on progression-free survival (PFS) and overall survival (OS) was also analyzed.⁴⁸ Before 3F8 treatment, 24 of 45 patients were in CR, 12 in VGPR and 9 in PR, according to International Neuroblastoma Staging System⁴⁹ criteria plus ¹³¹I-3F8 scan. 71% had detectable tumor cells in marrow by real-time RT-PCR. Of the 32 positive patients, 20 became negative after therapy, a 63% efficacy (Table 6).

TABLE 6
Tumor cell detection in the bone marrow by real-
time quantitative RT-PCR versus immunocytology and
histology during immunotherapy*

| | Before hot | |
|---|---|---|
| | antibody No. of | After hot + 1 cycle cold |
| Positivity by | patients (% Pos) | No. of patients (% Pos) |
| GD2 synthase real-time | 32/45 (71.1%) | 12/45 (26.7%) |
| Immunocytology | 8/41 (19.5%) | 9/44 (20.5%) |
| Histology | 0/45 (0%) | 4/45 (8.9%) |

*hot = ¹³¹I-3F8;
cold = unlabeled 3F8
GD2 Synthase/GAPDH < 5 was defined as negative.

When patients were stratified by CR/VGPR versus PR, GD2 synthase positivity was prognostic when detected before 3F8-targeted therapy (PFS, p=0.045 and OS, p=0.010). Persistent marker positivity was also predictive of PFS (p=0.035) and OS (p=0.027). (FIGS. 10A and 10B) Patients who succumbed to the disease had four times the level of transcript than those who remain alive (FIG. 7B). It is concluded that GD2 synthase mRNA is a useful surrogate marker for adjuvant treatment efficacy in NB with strong prognostic potential.

D. Research Design and Methods:

In the past decade, sequential clinical protocols were designed specifically for the treatment of metastatic neuroblastoma at MSKCC. At the time of minimal residual disease, two important adjuvant treatment strategies have been employed, autologous marrow/stem cell transplantation (BMT/SCT), and anti-GD2 monoclonal antibody 3F8 immunotherapy. Since 1987 all patients accrued on MSKCC protocols had their frozen tumors archived for biologic studies. They also underwent a comprehensive extent of disease workup (including marrow studies) at critical steps in their treatment, i.e. at diagnosis, before marrow or stem cell harvest, end of induction therapy, before and after adjuvant therapy (BMT/SCT or antibody), then every 3 months until the end of therapy (usually 2 years from diagnosis or from first treatment at MSKCC). Each marrow study consisted of 2 biopsies and 4 aspirates for histologic studies, plus heparinized marrow samples from all four aspirate sites for immunocytology. Unused mononuclear cells were cryopreserved for molecular marker studies. All marrow samples were obtained with parental consent and done under anesthesia in the outpatient department. Our objective in this grant application is to analyze specific cohorts of patients at specific steps during their treatment to determine whether molecular detection of MRD has clinical utility in measuring tumor response and in predicting survival among patients with high-risk neuroblastoma, i.e. those with stage, 4 or MYCN-amplified stage 3 diagnosed after their first birthday.

Specific Aim 1:

To evaluate and compare the efficacy of two important adjuvant therapies in neuroblastoma, marrow/stem cell transplantation (patient N=139) and anti-GD2 monoclonal antibody 3F8 immunotherapy (patient N=269) from a single institution. Cryopreserved marrows before and after these adjuvant therapies from a large consecutive patient cohort diagnosed with high-risk neuroblastoma spanning the past 14 years will be evaluated for MRD. Efficacy will be determined by the percentage of patients whose marrows which are positive by RT-PCR before treatment become negative following treatment, as well their magnitudes of response. MRD response will also be correlated with the level of pre-treatment tumor content. It is hypothesized that antibody treatment is as effective as marrow/stem cell transplant. It will be tested to see if clinical/biologic markers at diagnosis (ploidy, MYCN, histology, 1p36LOH, 17q gain, serum LDH and serum ferritin), known to impact on patient outcome, will correlate with MRD response of either adjuvant therapy. Additionally, host factors (ANC, ALC, serum complement) that mediate tumor killing by 3F8 will be correlated with efficacy of immunotherapy.

I. Marrow/Stem Cell Transplantation Study (Patient N=139)

The following transplant protocols will be included:

| Protocol | Preparative Regimen | Purged marrow | N |
|---|---|---|---|
| 87-56 | BCNU/Melphalan/CDDP/etoposide | 4-HC | 18 |
| 92-148 | Thiotepa/carboplatin/topotecan | None | 18 |
| 94-11 | (N7) 131-I-3F8 | 3F8 | 54 |
| 00-65 | (N8) Thiotepa/carboplatin/ | None | 49* |
| | topotecan | | |
| | | Total: | 139 |

*N8 is ongoing and the final expected accrual is 49.

IRB #87-56:

Myeloablative combination chemotherapy and local irradiation with autologous bone marrow rescue for neuroblastoma. Patients who had histologically-confirmed, initially extensive and unresectable, or relapsed NB were eligible. 3-4 weeks after the completion of at least two cycles of induction therapy, BM was harvested if it was clear of tumor cells. Marrow was purged 4hydro-peroxycylcophosphamide. Myeloablative combination chemotherapy consisted of BCNU, melphalan, carboplatin and etoposide. Local irradiation (2100 cGy) was done prior to BMT.

IRB #92-148:

Myeloablative chemotherapy with stem cell rescue for rare poor-prognosis cancers. Patients who had histologically-confirmed, stage 4 or MYCN amplified stage 3 NB were eligible. BM or peripheral blood stem cells was harvested during marrow remission. Marrow Was not purged. Preparative regimen consisted of thiotepa, carbolatin, and topotecan. Local irradiation (2100 cGy) to the primary site was generally done after engraftment.

IRB #94-011:

N7: Evaluation of maximal chemotherapy dose intensity plus monoclonal antibody 3F8 in the treatment of neuroblastoma. ¹³¹I-3F8 was used as preparative regimen prior to BMT. All marrows were purged with 3F8. Local radiation was administered prior to ¹³¹I-3F8 treatment.

IRE #00-65:

N8: Dose-intensive chemotherapy plus biologics in the treatment of neuroblastoma. In the ongoing N8, BM or peripheral blood stem cells was harvested during marrow remission without in vitro purging. Preparative regimen consists of thiotepa, carboplatin, and topotecan. Local irradiation (2100 cGy) to the primary site is done after engraftment.

II. Anti-GD2 Monoclonal Antibody 3F8 Immunotherapy Study (Patient N=269)

Each cycle of 3F8 treatment is standardized at 10 mg/m²/d for a total of 10 days. Marrow testing will include the first treatment cycle, as well as the cumulative efficacy of two, four and >four 3F8 cycles. The following immunotherapy protocols will be included:

| | | BM Re- | BM | | 3F8 Cycle: points of evaluation | | | |
|---|---|---|---|---|---|---|---|---|
| Protocol | Treatment | mission | Disease | N | #1 | #2 | #4 | >4 |
| 87-59 | 3F8 | — | 16 | 16 | x | | | |
| 87-118 | 3F8 | 14 | — | 14 | x | x | x | x |
| 89-175(N6) | 3F8 | 25 | 2 | 27 | x | x | x | x |
| 99-33 | 3F8 | 21 | — | 21 | x | | | |
| 94-011(N7) | 3F8 | 52 | 10 | 62 | x | x | x | x |
| | subtotal | 112 | 28 | 140 | | | | |
| 94-18 | 3F8 + GMCSF | 37 | 43 | 80 | x | x | x | x |
| 00-65 (N8) | 3F8 + GMCSF | 49* | — | 49* | x | x | | |
| | subtotal: | 86 | 43 | 129 | | | | |
| | TOTAL: | 198 | 71 | 269 | | | | |

*N8 is ongoing and 49 is the final expected accrual.

IRB #87-59:

Phase II trial of anti-ganglioside GD2 mouse monoclonal antibody 3F8 in the treatment of patients with neuroblastoma. This protocol was designed to determine the therapeutic efficacy of 3F8 for patients with measurable metastatic neuroblastoma.

IRB #87-118:

Phase II trial of anti-ganglioside GD2 mouse monoclonal antibody 3F8 as an adjuvant in the treatment of patients with the diagnosis of stage 4 neuroblastoma in second or subsequent remission. 3F8 was evaluated if it was efficacious in the eradication of microscopic disease, since 50-80% of stage 4 NB patients relapsed, despite supralethal doses of chemoradiotherapy together with autologous bone marrow rescue.

IRB #89-175:

N6: Evaluation of maximal dose intensity of chemotherapy and adjuvant mouse monoclonal antibody 3F8 in the treatment of neuroblastoma. The objectives were to increase the response rate as well as the disease-free survival of patients with poor-risk neuroblastoma. 3F8 was used as adjuvant after induction chemotherapy.

IRB #99-33:

Monoclonal antibody 3F8 and oral etoposide for the treatment of neuroblastoma. By means of standard imaging methods and tumor marker studies, the anti-tumor response and progression-free survival estimates among patients with high-risk NB undergoing periodic injection of 3F8 plus oral etoposide were determined. These patients were evaluable for response to 3F6 after the first cycle.

IRB #94-18:

Phase II trial of monoclonal antibody 3F8 and granulocyte-macrophage colony-stimulating factor (GM-CSF) for neuroblastoma. GM-CSF was used to mobilize host granulocytes for selective destruction of neuroblastoma and 3F8 for MRD eradication.

IRB #00-65:

N8: Dose-intensive chemotherapy plus biologics in the treatment of neuroblastoma. 3FB+GMCSF is given for a total of 2 cycles immediately following BMT or SCT. Patients are then continued on 3F8 as part of their adjuvant therapy.

Among these 269 patients, 71 had evidence of disease at the time of 3F8 treatment and obviously belonged to a higher risk group. Their level of disease when measured by RT-PCR is expected to be much higher. Of the 71 patients, 28 were treated with 3F8 alone, while 43 was treated with 3F8 +GMCSF. There will be an attempt to test if the response rates and the level of response (by quantitative RT-PCR) are different between antibody versus antibody plus GMCSF regimens. Among the 198 patients treated in remission, 112 were treated with 3F8 alone, while 86 with 3F8+GMCSF. Comparison of the quality of response between 3F8 versus 3F8+GMCSF can be made. The overall results will then be compared to the efficacy of BMT/SCT.

Experimental Methods:

In the detection of GAGE1 expression by RT-PCR and chemiluminescent detection, it was found that conventional detection using ethidium bromide has limited sensitivity. By using biotin-labeled GAGE1-specific primers and detection by chemiluminescence, the sensitivity and specificity has been vastly improved.^{39,40} Chemiluminescence is a chemical reaction in which alkaline phosphatase binds to substrate like CDP-Star, leading to a dephosphorylated substrate, which in turn has strong affinity to the hydrophobic sites on the nylon membrane, emitting light signal at 477 nm. This persistent light signal is imaged/recorded on film.

The GAGE1-specific forward primer, 5′-AGACGCTACGTAGAGCCT-3′ and reverse primer, 5′-CCATCAGGACCATCTTCA-3′ were both 5′-end labeled with biotin. PCR was performed in a thermal cycler (Hybaid, Franklin, Mass.). After a 12 min 94° C. activation of AmpliTaq Gold DNA polymerase (ABI Biosystems, Foster City, Calif.), 30 cycles of reaction was carried out with denaturation at 94° C. for 1 min, annealing at 60° C. for 2 min, and extension at 72° C. for 3 min. A final extension at 72° C. for 8 min completed the PCR reaction. PCR products were electrophoresed on 1.5% high-melting agarose gel (Fisher Scientific, Pittsburgh, Pa.) with biotin- labeled DNA molecular weight standards (BioVentures, Murfreesboro, Tenn.). The gel was visualized by ethidium bromide staining and the gene-specific product was 239 bp. The gel was transferred to Zeta-Probe nylon blotting membrane (Bio-Rad, Hercules, Calif.) by means of a semidry electrophoretic transfer cell from Bio-Rad. DNA was cross-linked by a UV crosslinker (Strategene, La Jolla, Calif.).

Chemiluminescent detection of membrane-bound DNA was carried out according to the Tropix Southern-light protocol (Bedford, Mass.). In brief, the procedure began with a blocking step using 0.2% I-Block (Tropix) in 1×PBS with 0.5% SDS. Upon incubation with Avidx-AP (Tropix) conjugate solution at 1:4000 dilution, the membrane was again placed in blocking buffer, followed by 3 washing steps with 1×PBS and 0.5% SDS. Chemiluminescent substrate CDP-Star (Tropix) at a final concentration of 80 uM in assay buffer (0.1M diethanolamine and 1 mM MgCl₂ with pH adjusted to 9.5 was added on the membrane, pressed between two transparencies. After a reaction of at least 30 minutes in the dark, the membrane was exposed to film. At least 500-fold increase in signal by chemiluminescence could be achieved.

Because of its exquisite sensitivity, once in a while, multiple non-specific bands were detected by chemiluminescence. In order to identify the correct amplified DNA product, GAGE1-specific probe that incorporated digoxigenin-11-UTP (Boehringer Mannheim, Germany) was designed. The forward and reverse primers were 5′-CGA GCA GTT CAG TGA TGA-3′, and 5′-ACC CTG TTC CTG GCT ATC-3′, respectively. The expected probe size was 154 bp. Membranes that had questionable chemiluminescent findings were hybridized with the GAGE1 probe overnight at 42° C., and proceeded with Southern blot according to the protocol by Boehringer Mannheim for the detection of digoxigenin-labeled nucleic acid by chemiluminescence. Briefly, membrane was transferred to blocking buffer that was 1% Blocking Reagent (Boehringer Mannheim) in maleic acid. A 1:4000 dilution of anti-digoxigenin-AP (Boehringer Mannheim) in blocking buffer was added to the membrane, followed by 3 washing steps with 1× maleic acid and 0.03% tween 20.

Although chemiluminescence provides sensitivity and southern blotting of electrophoretic PCR products ensures specificity, the current assay is qualitative and time-consuming. The advent of real-time quantitative PCR technology enables researchers with a new and potentially powerful tool to monitor minimal residual disease.⁵⁰⁻⁵² Instead of detecting the presence versus absence of a gene expression, one can monitor the level of target gene expression over time. With proper standardization, optimization, and validation, as well as the use of appropriate amounts of primers and probes for both the target gene as well as the endogenous control, multiplex experiments can be run with high sample throughput, good reproducibility, and no post-PCR handling or carryover contamination.

Real-Time Quantitative RT-PCR:

Background:

Relative quantitation of GD2 synthase m was achieved by means of the ABI Prism 7700 Sequence Detection System (Applied Biosystems, Foster City, Calif.). In TaqMan real-time quantitation technology, the 5′exonuclease activity of the Taq polymerase cleaves and releases the hybridization probe that was labeled with a fluorescent reporter dye. This fluorogenic probe is specific for the target sequence, thereby generating a fluorescence signal that is specific and is directly proportional to the amount of PCR product synthesized. PCR reactions are characterized by the time-point during cycling when amplification of the PCR product is first detected, rather than the amount of product accumulated after a fixed number of cycles. Since the amount of product at the exponential phase of the PCR is proportional to the initial copy number of the target, the more abundant the starting quantity of a target, the earlier will the PCR amplification be detected by means of the fluorescence signal. In this technology, the target quantity is measured by identifying the threshold cycle number (CT), i.e. when the fluorescence signal crosses a preset detection threshold. The laser detector of the Prism 7700 monitors the cycle-to-cycle change in fluorescent signal on-line. The fewer cycles it takes to reach a detectable level of fluorescence, the greater the initial copy number.

Measurement of GD2 synthase transcript was based on two reporter dyes with the largest difference in emission wavelength maxima, namely 6-FAM for GD2 synthase and VIC for glyceraldehyde-3-phosphate dehydrogenase (GAPDH), our endogenous reference to control for difference in mRNA extraction and cDNA synthesis. After optimization by limiting the primer concentrations of the more abundant target GAPDH, multiplex PCR became the standard assay, resulting in higher throughput and reducing the effect of pipetting errors.

The primers and probe for GD2 synthase were designed using the applications-based primer design software, Primer Express (Applied Biosystems, ABI). The probe spanned an intron, thereby avoiding the amplification of contaminating genomic DNA present in the sample. GD2 synthase sense primer was 5′-GACAAGCCAGAGCGCGTTA-3′, antisense primer was 5′-TACTTGAGACACGGCCAGGTT-3′. Probe was FAM-5′AACCAGCCCTTGCCGAAGGGC-3′ (99 bp). GAPDH sense primer was 5′-GAAGGTGAAGGTCGGAGTC-3′, and antisense primer was 5′-GAAGATGGTGATGGGATTTC-3′. Probe was VIC-5′CAAGCTTCCCGTTCTCAGCC-3′ (226 bp). GD2 synthase and GAPDH designs were based sequence from GenBank, accession NM_{—}001478 and J04038, respectively. Primers and probes were synthesized by ABI.

Procedure:

In each 25 ul MicroAmp optical tubes (ABI), 2 ul cDNA template was added to a PCR mix. This mixture included the Taqman master mix (ABI) containing 5 mM MgCl₂, 200 uM each dATP, dCTP, dGTP, and dUTP, 0.05 U/ul AmpliTaq Gold DNA polymerase, as well as 0.01 U/ul AmpErase uracil-N-glycosylase (UNG) to prevent PCR product carryover, as well as a passive reference dye ROX. This reference dye provides an internal reference to which the reporter dye signal can be normalized, compensating for the fluorescence between wells and between experiments caused by pipetting errors or instrument variability. Also included in the mix was 300 nM each GD2 synthase forward and reverse primers, 200 nM GD2 synthase (FAM) probe, and 40 nM each GAPDH primers and 100 nM GAPDH (VIC) probe. Each tube was covered with a MicroAmp optical cap. Every PCR run included a 5-point standard to generate a standard curve for GD2 synthase and for GAPDH, plus a no template control. Samples were often run in duplicate PCR experiments.

Using the ABI Prism 7700 Sequence Detector, the initial PCR began with a 50° C., 2 minutes step to optimize UNG activity, followed by a 95° C., 10 minutes step to activate AmpliTaq Gold DNA polymerase and UNG deactivation. Then 40 cycles at 95° C. for 15 seconds and 60° C. for 1 minute followed. The entire PCR took 2 hours to complete with no post-PCR handling.

Calculation:

For each unknown test sample, the amount of GD2 synthase and endogenous reference GAPDH was determined from the respective standard curve. Dividing the GD2 synthase level by the GAPDH level resulted in a normalized GD2 synthase value. Quantitation of 31 normal bone marrow and peripheral blood established the threshold below which the quantitative value was considered background. The variation in the quantitation from experiment to experiment was within 15%.

Statistical Analysis for Aim 1:

The first goal of this proposal is to use two uniquely different molcular markers to evaluate the efficacy of two important adjuvant therapies, BMT/SCT (139 patients) versus 3F8 immunotherapy (269 patients), employed in the treatment of high-risk neuroblastoma patients. In order to determine MRD response (i.e. efficacy) in the bone marrows, two measurements will be recorded at two time points, namely before and after therapy. The first measurement utilizes RT-PCR of GAGE1 with dichotomous outcome. The second measurement is real-time quantitative RT-PCR of GD2 synthase with continuous outcome. Efficacy will be determined by the percentage of patients whose marrows which are positive by RT-PCR before treatment become negative following treatment, as well their magnitudes of response. MRD response will also be correlated with the level of pre-treatment tumor content. It is hypothesized that antibody treatment is as effective as marrow/stem cell transplant. It will be tested to see if clinical/biologic markers at diagnosis (ploidy, MYCN, 1p36LOH, 17 q gain, serum LDH and serum ferritin), known to impact on patient outcome, will influence MRD response. of either adjuvant therapy. Additionally, host factors (ANC, ALC, serum complement) that mediate tumor killing by 3F8 will also be correlated with efficacy of immunotherapy.

I. To evaluate efficacy in tumor cell depletion (MRD response) within each treatment group.

a). The probability that a pre-treatment positive sample turns negative as detected by GAGE1 can be calculated by using sample proportion. The corresponding 95% confidence interval can be determined. Comparisons between BMT/SCT and 3F8 immunotherapy can be done by using Fisher's exact test or chi square test.

b) . With GD2 synthase data, let y1 and y2 be the RT-PCR measurements before and after treatment. Also let d=(y1−y2)/y1 be % response. By plotting y1 versus d, it can be determined if % response is inversely correlated with the pre-treatment tumor content as determined by these two molecular markers. We will try to fit a nonlinear model. To compare % response in BMT/SCT and antibody 3F8 group, the equality of two parameters in these two nonlinear models can be tested.

II. To test if MRD response is influenced by established clinical/biologic markers at diagnosis in both treatment groups, or host factors in antibody treatment group.

Let D1 and D2 be the RT-PCR findings of GAGE1 before and after treatment and y be the difference in RT-PCR measurements of GD2 synthase before and after treatment. Let D1=1 if the GAGE1 result is positive and 0 if it is negative. The same definition is applied to D2. For both treatment groups, a parametric joint distribution assumption can be made for the distribution of (D2,y) given covariate D1 and x, where x are the measurements of established biologic markers at diagnois, including ploidy, MYCN, histology, 1p36LOH, 17 q gain, serum LDH and serum ferritin. Marginal generalized linear models for P(D2=1|x,D1) and P(y|x,D1) can also be postulated. Estimation of the underlying parameters will be based on longitudinal data analysis of Liang and Zeger⁵³ as well as Fitzmaurice and Laird.⁵⁴ The same methodology can be applicable to antibody 3F8 group, where the covariates will also include ANC, ALC, and serum complement at time of treatment.

Potential Pitfalls of Aim 1:

The specimens for this study will all be cryopreserved samples, some being stored for 14 years. Poor quality mRNA will be excluded from our analyses. Our experience on 2000 samples studied so far showed that 3% were excluded because of poor mRNA quality. The cDNAs prepared were stored at −80° C. and they can be used for multiple molecular markers. Even though our sample size will be relatively small, it is considered quite large for a single institution study. Our clinical data on individual patients, including follow-ups are comprehensive.

False negative PCR reactions are most likely of a technical nature, such as omission of one of the reagents, incomplete thawing or mixing of frozen reagents, malfunctioning pipettes or poor mRNA. The inclusion of a positive (housekeeping genes) control, e.g. β2 microglobulin or GAPDH is used to ensure both mRNA integrity plus functional RT-PCR. Inhibitors of PCR reactions in reactive cells (or inflammatory debris) or heparin can diminish PCR sensitivity. To avoid such interference, the cell fraction is first separated by Ficoll centrifugation (our standard method). The inhibitory effect of heparinized plasma could also be efficiently removed by extracting with Celite, which has so far been unnecessary. False negativity can also result from tumor clones that do not express the marker of interest. The use of multiple markers can potentially overcome such problems. Ultimately the limit will be reached if there is <1 tumor cell in the sample.

False positive RT-PCR results from contamination of samples or reagents, preventable by careful laboratory techniques. These include frequent glove changes, use of aliquoted reagents, plugged pipette tips, reduction of pipetting steps by using reagent mixes. An important consideration is to keep the pre and post PCR reactions separate so as to avoid contamination, e.g. by aerosol. In addition, every PCR reaction should be run with “no DNA template” control. When false positives occur, a series of negative controls (in which one component of the PCR reaction is replaced by the same reagent from another aliquot or lot) should be tested to pin-point source of contamination. Sometimes the pre-PCR area need to be decontaminated by overnight exposure to UV light, and apparatus washed with 0.1N HCl before autoclaving in deionized water. Chemiluminescence was chosen over nested PCR to improve sensitivity and to avoid potential for false positives.

Illegitimate transcription is another potential cause of false positivity. Although the amount of illegitimate transcripts in usually low, estimated at 1 mRNA molecule per 100 to 1000 cells, it can result in false positives by RT-PCR. Since illegitimate transcription of multiple markers simultaneously in the same cell is of lower probability, the use of at least 2 markers to determine positivity will reduce this problem. Pseudogenes can also give false-positive signals because they lack the intronic sequence and their PCR product will be identical in size to amplified PCR products of cDNA. This type of false positives can be controlled by amplification of human genomic DNA without RT-step. Alternatively, the use of multiple markers on the same sample to determine positivity can also overcome this pitfall. Our choice of cancer-testis antigen GAGE1 should reduce the issue of of tissue cross-reactivity given its restricted pattern of expression.

Potential pitfalls of quantitative RT-PCR have been extensively researched. While the real-time quantitative PCR offered advantages over other conventional methods, the efficiency of mRNA extraction and reverse-transcription are variables that need to be strictly quality-controlled. For GD2 synthase the results of real-time quantitative RT-PCR were validated by correlating with immunocytology. Molecular marker GAGEI is expected to be optimized by designing special primers and probes for a real-time RT-PCR assay. This will provide us a second quantitative molecular probe.

Anticipated Significance of Aim 1:

In the BMT/SCT study, the following will be tested: the efficacy of the transplant procedure, which is a composite of the level of residual disease in the body, the myeloablative regimen used, and the quality (level of tumor contamination) of the marrow or stem cell infused. Based on our recent studies^{47,48} (see preliminary results), the presence of MRD has far more clinical relevance than efficiency of ex vivo purging. With the exception of N7, all the transplant protocols were similar in the use of high-dose chemotherapy, rendering them comparable as a group in the testing of efficacy. By default these pre-transplant marrows would be “remission” marrows by histology. However, since the molecular markers proposed to be useed are far more sensitive than histology, a substantial number of pre-transplant samples are expected to be detectable, permitting an evaluation of change in MRD. In the immunotherapy group, since most patients received repeated cycles of 3F8, serial samples would provide substantially more information in correlation with MRD response.

Using these molecular markers, two types of results, both qualitative and quantitative, will be achieved. As a qualitative measure, the percentage of patients whose marrow turned from positive to negative for each adjuvant treatment, BMT/SCT, 3F8, and 3F8+GMCSF, can be estimated and compared. The quantitative response, i.e., percent tumor reduction with treatment can also be ascertained. The efficacy of each modality will likely depend on the pre-treatment tumor content, i.e. molecular units of MRD based on quantitative RT-PCR. For comparison among treatment modalities, patients can also be stratified according to pretreatment tumor content. Clearly these studies focus on marrow disease, and conclusions may not necessarily reflect the entirety of disease content in the patients. But there is reason to believe that metastatic neuroblastoma is a systemic disease. And like leukemia, marrow assessment is a close approximation of the overall disease state. If these molecular methods prove to be sensitive, they will provide a useful tool for measuring circulating tumor cells in marrow as the patient goes through various phases of treatment. They will allow comparison between phases and among patients. With the promise of new therapeutics directed at novel molecular targets, many of which are most likely to act best at the time of minimal clinical disease, molecular monitoring will provide a powerful tool to evaluate such strategies objectively. In pediatric cancers where randomized studies are difficult to carry out because of the small number of affected patients, such tools hold even greater utility in rapidly arriving at the optimal drug and their optimal use in the curative treatment of this disease.

Specific Aim 2:

To test the prognostic significance of MRD on progression-free survival and overall survival in a cohort of unselected consecutive and newly diagnosed stage 4 neuroblastoma patients ranging from 1-17 years of age (patient N=130) who were treated on consecutive MSKCC protocols N5, N6, N7, N8 evolving over the last 14 years. MRD from their marrows as detected by RT-PCR of GD2 synthase mRNA and GAGE1 will be evaluated at diagnosis, end of induction (right before transplant or 3F8 treatment), at 12, 18, and 24 months from diagnosis. Based on our preliminary findings, it is hypothesized that MRD at the end of induction and at 2 years from diagnosis will have the strongest prognostic impact on patient survival.^{44,46,48} will also test if MRD has an independent prognostic impact in predicting patient outcome, when known clinical, biologic, and treatment variables (serum ferritin, serum LDH, tumor histology (Shimada classification), MYCN amplification, chromosome 1p36LOH, 17 q gain and the presence of anti-idiotype network) are included in the multivariate model. It is hypothesized that MRD, being the final common pathway of inadequately treated or resistant cancer, will be a dominant determinant of outcome, as adverse tumor biology continues to be neutralized by increasingly effective treatment strategies. Patients in the following protocols will be included.

| Protocol | Patients |
|---|---|
| 87-97 N5 | 26 |
| 89-175 N6 | 29 |
| 94-011 N7 | 41 |
| 00-65 N8 | 34 |
| Total | 130 |

*Of the 49 accruals in N8, 34 will be newly diagnosed patients.

These protocols were designed to test dose-intensive chemotherapy, surgery, hyperfractionation radiation, and monoclonal antibody 3F8 (unlabeled and ¹³¹I-labeled) in the comprehensive therapy of neuroblastoma. These stage 4 patients had high-risk clinical and biologic markers. Evaluations of sequential endpoints, namely primary tumor resectability, overall response, and progression-free survival compared favorably with predictions. There were no late relapses after 3 years from diagnosis.

Protocol N5 used high-dose chemotherapy (cyclophosphamide, cisplatin and epipodophyllotoxin, cisplatin/VP16), while N6 utilzed maximal dose intensive chemotherapy and adjuvant 3F8 immunotherapy. N7 built on N6's aggressive multi-modality approach with the addition of ¹³¹I-3F8 as part of the treatment plan for poor-risk neuroblastoma patients, while N8 reintroduces chemotherapy-based ABMT. The rationale is that among these patients, treatment-related leukemia (t-AML) and isolated central nervous system (CNS) relapse have emerged as new obstacles, accounting to about one-half of all relapses. It is reasoned that myeloablative therapy can eliminate leukemic clones if the marrow or a healthy stem cell harvest can be obtained early in induction. Moreover, high dose therapy such as thiotepa, carboplatin, and topotecan can penetrate the blood-brain barrier, and may be useful against MRD in the CNS, which has remained a sanctuary site for NB cells, since the blood-brain barrier renders both systemic chemotherapy and immunotherapy ineffective. N8 was designed to minimize exposure to chemotherapy by reducing the number of cycles of induction chemotherapy from seven to five, and to optimize CNS prophylaxis. Cis-retinoic acid was also incorporated in the protocol. Our single institution experience is that N5 patients achieved PFS of 25%, while 40% PFS was seen with N6 patients; >50% with N7 patients, and >60% expected among patients treated with the ongoing N8.

Statistical Analysis for Aim 2:

The prognostic importance of MRD will be evaluated in a cohort of unselected consecutive and newly diagnosed stage 4 neuroblastoma patients ranging from 1-17 years of age (N=130) who were treated on consecutive neuroblastoma protocols. Their marrows will be evaluated at diagnosis, end of induction (right before transplant or 3F8 treatment), 12, 18 and 24 months from diagnosis for occult tumor cells by GAGE1 and GD2 synthase RT-PCR. The following statistical analysis will be performed.

I. Univariate Survival Analysis.

The correlation between MRD, clinical, biological, and treatment variables with progression-free survival and overall survival will be examined by using the log-rank statistics or univariate Cox model depending on whether the examined variable is discrete or continuous. These variables include serum ferritin, serum LDH, tumor histology (Shimada classification), MYCN amplification, chromosome 1p36LOH, 17 q gain and the presence of anti-idiotype network.

II. Multivariate Survival Analysis.

Variables with a significant level of less than 0.1 in univariate survival analysis will enter into multivariate analysis. It is very important to adjust for known clinical and related biological factors since the MRD as determined by these molecular markers are likely to be correlated with existing clinical factors. It will be very useful to determine whether MRD by molecular detection can provide additional prognostic information over and above what is already available in the clinical factors. A strong association found between marker positivity and survival, both progression-free and overall will suggest that these markers have potential clinical utility.

Multivariate Cox regression analysis will then be performed separately by using the collected covariate information at different time points, namely at diagnosis, end of induction, 12, 18 and 24 months after diagnosis. This analysis will allow us to determine whether MRD indeed has an independent prognostic impact on patient survival. Time dependent Cox regression model will also be used to assess overall and progression free survival by taking into account the change in RT-PCR findings at different time points.

III. To correlate multiple measurements of GAGE1 and GD2 synthase results with clinical/biologic variables.

Let D₁, D₂, D₃, D₄, D₅ be RT-PCR measurements of GAGE1 at diagnosis, end of induction (right before transplant or 3F8 treatment), 12, 18 and 24 months from diagnosis. Similarly let y₁, y₂, y₃, y₄, y₅ be RT-PCR measurements of GD2 synthase for the same time points. Let x be the covariates as specified in I. It can be assumed that a logistic regression model for D₁, D₂, D₃, D₄, D₅ for a given covariate x as

$$P ( {D}_{i} = 1 \vert x ) = \frac{exp ( {a}_{i} + {b}_{i} x ) .}{1 + exp ( {a}_{i} + {b}_{i} x )} , i = 1 , 2 , 3 , 4 , 5$$

It can also be postulated that a parametric model for yᵢ conditional on Dᵢ and x

f(yᵢ|x,dᵢ)=f(yᵢ|x,dᵢ, qᵢ), i=1,2,3,4,5

where aᵢ, bᵢ, qᵢ are unknown parameters. Since RT-PCR results before and after treatments are correlated, longitudinal data analysis can be done.^{53,54}

IV. To find the functional relationship between RT-PCR with time.

There is also an interest in studying the detection (GAGE1) and quantitation (GD2 synthase) of these molecular markers as a function of time.

Denote the baseline level (at diagnosis) as x₀. Let y₁=x₁−x₀, y₂=x₂−x₀ and y₃=x₃−x₀ be the differences from the baseline level at 12, 18, and 24 months from diagnosis. We would like to model this as

yᵢ=f(tᵢ, q)+eᵢ

where t₁=0, t₂=0.5, t₃=1 denotes calendar time and e is the unobservable error. Different models for f will be considered. A possible model is the nonlinear growth model.

f(tᵢ, q)=exp(qᵢ+q₂tᵢ)

Nonlinear regression techniques can be applied for finding the unknown parameter q and testing the validity of the proposed model.

Potential Pitfall in Statistical Analysis:

Non-ignorable missing data. This is a challenging statistical problem because of the fact that RT-PCR measurements will be tested at different time points, and there may likely be losses in the numbers of patients available for testing due to relapse/death at times points further away from diagnosis. Statistical analysis will be based on works by Lipsitz et al,⁵⁵ and Troxel et al.⁵⁶ Survival analysis will also be performed by taking into consideration potential missing covariate data.⁵⁷⁻⁵⁹

Anticipated Significance of Aim 2:

Patient marrows will be tested at pre-determined time points. From our study using the marker GAGE1,⁴⁴ with a cohort of N6 and N7 patients, approximately 30% of patients were positive at 12 months, 18 months, and 24 months after diagnosis. However, only marrows sampled at 24 months were associated with adverse survival status with statistically significance. Our recent work indicated that post-induction persistent disease can also have serious adverse consequence.⁴⁸ RT-PCR results on samples drawn at diagnosis would also allow us to compare with other multi-institutional experience²⁰ regarding the relationship between positive marrow at diagnosis and poor patient outcome. In this aim, our sample size of 130 patients is quite large for a single institution study. Comprehensive clinical data on individual patients is available, including follow-ups. Since advanced stage neuroblastoma has such a poor prognosis, minimal residual disease may represent the final pathway of biology and treatment, ultimately assuming a crucial role in determining patient's eventual clinical outcome. Our goal is to identify the time point(s) when MRD in the bone marrow as determined our two molecular markers will have the most prognostic impact on patient survival.

Since patients are in apparent clinical remission, these markers will potentially serve as objective endpoints for stopping further treatment, and serve as sensitive surrogates for evaluating adjuvant therapies.

E. Human Subjects:

Protection of Human Subjects: For ongoing treatment protocols, including 94-18, 99-33, and 00-65 (N8) bone marrow samples from patients will be collected according to the treatment plans. The risks to the subjects are acceptable in relation to the anticipated benefits to the subjects and in relation to the importance of knowledge expected to be gained. The proposed research project will involve the use of marrows from human subjects. The marrow obtained from patients remaining after clinically indicated assays, as well as obtained in limited amounts (10 cc, <5% blood volume, <5% bone marrow volume) for research purposes following informed consent under the guidelines of MSKCC IRB approved protocols, will be used. Risks to the participants are the minimal risk associated with venipuncture. Most of the samples required in this study have already been collected and cryopreserved. The confidentiality of all participants will be protected. This is a diagostic study for human patients with a rationale built on encouraging preliminary data. Human subjects are required because this is a human cancer and the aim of the study is to design better ways to detect and cure this disease.

Inclusion of Women, Minorities, and Children:

This study is open to all patients with neuroblastoma. It includes both genders of all ages and all minorities. The incidence of neuroblastoma is similar among the sexes and among the different races. However, the ethnic mix among patients treated at MSKCC is dependent on the referral pattern in the greater metropolitan area. Patients of both sexes and of all ethnic backgrounds (Caucasian, Black, Hispanic, Asian, etc) are eligible for the study.

F. Vertebrate Animals:

Not applicable since no animal experiments are proposed.

### REFERENCES

1. Moss T J, Sanders D G, Lasky L C, Bostrom B: Contamination of peripheral blood stem cell harvests by circulating neuroblastoma cells. Blood 76:1879-1883, 1990

2. Brenner M K, Rill D R, Moen R C: Gene marking to trace origin of relapse after autologous transplants of relapse after autologous bone marrow transplantation. Lancet 341:85-86, 1993

3. Rill D R, Santana V M, Roberts W M, et al: Direct demonstration that autologous bone marrow transplantation for solid tumors can return a multiplicity of tumorigenic cells. Blood 84:380-3, 1994

4. Miyajima Y, Horibe K, Fukada M, et al: Sequential detection of tumor cells in the peripheral blood and bone marrow of patients with stage IV neuroblastoma by the reverse transcription-polymerase chain reaction for tyrosine hydroxylase mRNA. Cancer 77:1214-1219, 1996

5. Ladenstein R, Philip T, Lasset C, et al: Multivariate analysis of risk factors in stage 4 neuroblastoma patients over the age of one year treated with megatherapy and stem-cell transplantation:A report From the European Bone Marrow Transplantation Solid Tumor Registry. J Clin Oncol 16:953-965, 1998

6. Diel I J, Kaufmann M, Costa S D, et al: Micrometastatic breast cancer cells in bone marrow at primary surgery: prognostic value in comparison with nodal status. J Natl Cancer Inst 88:1652-8, 1996

7. Moss T J, Cooper B, Kennedy M J, et al: The prognostic value of immunocytochemical (IHC) analysis on bone marrow (BM) and stem products (PBSC) taken from patients with stage IV breast cancer undergoing autologous transplant therapy. Proc Amer Soc Clin Oncol 16:90a, 1997

8. Shpall E J, Jones R B: Release of tumor cells from bone marrow. Blood 83:623-5, 1994

9. Brugger W, Bross K J, Glatt M, et al: Mobilization of tumor cells and hematopoietic progenitor cells into peripheral blood of patients with solid tumors. Blood 83:636-40, 1994

10. Moss T J, Reynolds C P, Sather H N, et al: Prognostic value of immunocytologic detection of bone marrow metastases in neuroblastoma. N Engl J Med 324:219-226, 1991

11. Cortes J, Fayad L, O'Brien S, et al: Persistence of peripheral blood and bone marrow blasts during remission induction in adult acute lymphoblastic leukemia confers a poor prognosis depending on treatment intensity. Clin Cancer Res 5:2491-2497, 1999

12. Mattano L A, Moss T J, Emerson S G: Sensitive detection of rare circulating neuroblastoma cells by the reverse transcriptase-polymerase chain reaction. Cancer Res 52:4701-4705, 1992

13. Gilbert J, Norris M D, Marshall G M, Haber M: Low specificity of PGP9.5 expression for detection of micrometastatic neuroblastoma. Br J Cancer 75:1779-1781, 1997

14. Smith M R, Biggar S, Hussain M: Prostate-specific antigen messenger mRNA is expressed in non-prostate cells: Implications for detection of micrometastases. Cancer Res 55:2640-44, 1995

15. Schoenfeld A, Luqmani Y, Smith D, et al: Detection of breast cancer micrometastases in axillary lymph nodes by using polymerase chain reaction. Cancer Res 54:2986-2990, 1994

16. Young CYF, Montgomery B T, Andrews P E, et al: Hormonal regualation of prostate-specific antigen messenger mRNA in human prostatic adenocarcinoma cell line LNCaP. Cancer Res 51:3748-3752, 1991

17. Lode H N, Bruchelt G, Seitz G, et al: Reverse transcriptase-polymerase chain reaction analysis of monoamine transporters in neuroblastoma cell lines: correlations to meta-iodobenzylguanidine uptake and tyrosine hydroxylase gene expression. Eur J Cancer 31A:586-590, 1995

18. Burchill S A, Lewis I J, Abrams K R, et al: Circulating neuroblastoma cells detected by reverse transcriptase polymerase chain reaction for tyrosine hydroxylase mRNA are an independent poor prognostic indicator in stage 4 neuroblastoma in children over 1 year. J Clin Oncol 19:1795-1801, 2001

19. Fukuda M, Miyajima Y, Miyashita Y, Horie K: Disease outcome may be predicted by molecular detection of minimal residual disease in bone marrow in advanced neuroblastoma: a pilot study. J Pediatr Hematol Oncol 23:10-13, 2001

20. Seeger R C, Reynolds C P, Gallego R, et al: Quantitative tumor cell content of bone marrow and blood as a predictor of outcome in stage IV neuroblastoma: a Children's Cancer Group study. J Clin Oncol 18:4067-4076,2000

21. Kushner B H, Gulati S C, Kwon J-H, et al: High-dose melphalan with 6-hydroxydopamine-purged autologous bone marrow transplantation for poor-risk neuroblastoma. Cancer 68:242-247, 1991

22. Kushner B H, O'Reilly R J, Mandell L R, et al: Myeloablative combination chemotherapy without total body irradiation for neuroblastoma. J Clin Oncol 9:274-279, 1991

23. Kushner B H, O'Reilly R J, LaQuaglia M, Cheung N K: Dose intensive use of cyclophosphamide in ablation of neuroblastoma. Cancer 66:1095-1100, 1990

24. Cheung N K, Lazarus H, Miraldi F D, et al: Ganglioside GD2 specific monoclonal antibody 3F8- a phase I study in patients with neuroblastoma and malignant melanoma. J Clin Oncol 5:1430-1440, 1987

25. Cheung N K, Yeh S D J, Gulati S, et al: 1311-3F8 targeted radiotherapy of neuroblastoma (NB): a phase I clinical trial. Proc Am Assoc Cancer Res 31:284, 1990

26. Wu Z, Schwartz E, Seeger R C, Ladisch S: Expression of GD2 ganglioside by untreated primary human neuroblastomas. Cancer Res 46:440-443, 1986

27. Kramer K, Gerald W, Kushner B H, et al: Disialoganglioside GD2 loss following monoclonal antibody therapy is rare in neuroblastoma. Clin cancer Res 4:2135-2139, 1998

28. Cheung N K, Heller G, Kushner B H, et al: Detection of metastatic neuroblastoma in bone marrow: when is routine marrow histology insensitive? J Clin Oncol 15:2807-2817, 1997

29. Faulkner L B, Tintori V, Tamburini A, et al: High-sensitivity immunocytologic analysis of neuroblastoma cells in paired blood and marrow samples. J Hematother 7:361-366, 1998

30. Cheung N K, Von Hoff DD, Strandjord S, Coccia PF: Detection of neuroblastoma cells in bone marrow using G_{D2} specific monoclonal antibodies. J Clin Oncol 4:363-369, 1986

31. Van der Bruggen P, Traversari C, Chomez P, et al: A gene encoding an antigen recognized by cytolytic T lymphocytes on a human melanoma. Science 254:1643-1647, 1991

32. van den Eynde B. Peeters O, De Backer O, et al: A new family of genes coding for an antigen recognized by autologous cytolytic T lymphocytes on a human melanoma. J Exp Med 182:689-698, 1995

33. Boel P, Wildmann C, Sensi M, et al: BAGE, a new gene encoding an antigen recognized on human melanomas by cytolytic T lymphocytes. Immunity 2:167-175, 1995

34. Chen Y-T, Scanlan M J, Sahin U, et al: A testicular antigen aberrantly expressed in human cancers detected by autologous antibody screening. Proc Natl Acad Sci USA 94:1914-1918, 1997

35. Tureci O, Sahin U, Schobert I, et al: The SSX-2 gene, which is involved in the t(X;18) translocation of synovial sarcomas, codes for the human antigen HOM-MEL-40. Cancer Res.56:4766-4772, 1996

36. Boon T, Cerottini J C, Van den Eynde B, et al: Tumor antigens recognized by T lymphocytes. Ann Rev Immunol 12:337-365, 1994

37. Sahin U, Tureci O, Schmitt H, et al: Human neoplasma elicit multiple immune responses in the autologous host. Proc Nat Acad Sci 92:11810-11813, 1995

38. De Plaen E, Arden K, Traversari C, et al: Structure, chromosomal localization, and expression of 12 genes of MAGE family. Immunogenetics 40:360-369, 1994

39. Cheung I Y, Barber D, Cheung N K V: Detection of microscopic neuroblastoma in marrow by histology, immunocytology, and RT-PCR of multiple molecular markers. Clin Cancer Res 4:2801-2805, 1998

40. Cheung I Y, Cheung N K V: Molecular detection of GAGE1 expression in peripheral blood and bone marrow: utility as a tumor marker for neuroblastoma. Clin Cancer Res 3:821-826, 1997

41. Chi S N, Cheung I Y, Cheung N K V: SSX-2 and SSX-4: cancer-testis antigens as tumor markers of neuroblastoma. J Pediatr Hematol Oncol 22:380, 2000

42. Cheung I Y, Cheung N K V, Ghossein R A, et al: Association between molecular detection of GAGE1 and survival in patients with malignant melanoma: A retrospective cohort study. Clin Cancer Res 5:2042-2047, 1999

43. Karpoff H, Ghossein R, Cheung I, et al: Tyrosinase (TYR) and GAGE1 mRNA in peripheral blood (PB) and bone marrow (BM) predict outcome in stage III melanoma patients rendered free of disease. Proc Soc Surg Oncol p.19, 1999

44. Cheung I Y, Chi S N, Cheung N K V: Prognostic significanceof GAGE1 detection in bone marrows on survival of patients with metastatic neuroblastoma. Med Pediatr Oncol 36:632-634, 2000

45. LoPiccolo M S, Cheung N K V, Cheung I Y: GD2 Synthase: a new molecular marker for detecting neuroblastoma. Cancer 92:924-31, 2001

46. Cheung I Y, Cheung N K V: Quantitation of marrow disease in neuroblastoma by real-time RT-PCR. Clin Cancer Res 7:1698-1705, 2001

47. Cheung I Y, LoPiccolo M S, Collins N, et al: Quantitation of GD2 synthase mRNA by real-time reverse transcription-PCR: Utility in bone marrow purging of neuroblastoma by anti-GD2 antibody 3F8. Cancer 2002, in press.

48. Cheung I Y, LoPiccolo M S, Kushner B H, et al: Quantitation of GD2 synthase mRNA by real-time reverse transcription-PCR: Clinical utility in evaluating adjuvant therapy in neuroblastoma. Submitted for publication.

49. Brodeur G, Pritchard J, Berthold F, et al: Revisions of the international criteria for neuroblastoma diagnosis, staging and response to treatment. J Clin Oncol 11:1466-1477, 1993

50. Pongers-Willemse M J, Verhagen O J H M, Tibbe G J M, et al: Real-time quantitative PCR for the detection of minimal residual disease in acute lymphoblastic leukemia using junctional region specific TaqMan probes. Leukemia 12:2006-2014, 1998

51. Gerard C J, Olsson K, Ramanathan R, et al: Improved quantitation of minimal residual disease in multiple myeloma using real-time polymerase chain reaction and plasmid-DNA complementarity determining region III standards. Cancer Res 58:3957-3964, 1998

52. Niesters H G, van Esser J, Fries E, et al: Development of real-time quantitative assay for detection of epstein-barr virus. J Clin Microbiol 38:712-715, 2000

53. Liang K Y, Zeger S L: Longitudinal data analysis using generalized linear models. Biometrika 73: 13-22, 1986

54. Fitzmaurice G, Laird N M: Regression models for a bivariate discrete and continuous outcome with clustering. J Amer Statistical Assoc 90:845-852, 1995

55. Lipsitz S R, Ibrahim J G, Zhao L P: A weighted estimating equation for missing covariate data with properties similar to maximum likelihood. J Amer Statistical Assoc 94:1147-1160, 1999

56. Troxel A B, Lipsitz S R, Brennan T A: Weighted estimating equations with nonignorably missing response data. Biometrics 53: 857-869, 1997

57. Lin D Y, Ying Z: Cox regression with incomplete covariate measurements. J Amer Statistical Assoc 88: 1341-1349, 1993

58. Paik M C, Tsai W Y: On using the Cox proportional hazards model with missing covariates. Biometrika 84:579-593, 1997

59. Chen H Y, Little R J A: Proportional hazards regression with missing covariates. J Amer Statistical Assoc 94:896-908, 1999

Sixth Series of Experiments

WT-1, Midkine, GAGE1, and BAGE Markers

In order to determine the efficacy of adjuvant treatment on minimal residual disease (MRD), an endpoint or some benchmark of evaluation is essential. Survival endpoint has been the gold standard since MRD is beyond the sensitivity of conventional radiographic or histologic methods. However, in adjuvant study design, large patient cohorts and long followups are required. With the increasing availability of treatment choices (e.g. biologic and genetic treatments), rapid and patient-number sparing surrogate markers are necessary. This is particular important for rare diseases such as childhood cancers. Although large clinical trials have statistical power, they may not be practical in piloting exploratory strategies to identify useful drugs. In addition, since the maximum tolerated dose is often not the optimal biologic dose, standard phase I and II strategies may be unnecessarily toxic for optimizing biologic therapies. The ideal tumor markers of MRD should be tumor specific. Moreover, if immunologically-silent tumor antigens are shielded from the immune surveillance by being intracellular, poorly degraded, or not presentable by HLA molecules, they too can be ideal candidates as markers of MRD. WT-1, midkine, GAGE1 and BAGE are potential tumor markers for MRD.

Wilm's tumor suppression gene WT1 is overexpressed among leukemic blasts and desmoplastic small round cell tumor. Since it is not expressed in normal marrow or blood, it may be a useful marker of MRD. Midkine also known as NEGF2, is a heparin-binding growth factor identified as a product of a retinoic acid-response gene. It is a target gene for the Wilms' tumor suppression genes (WT1) and is highly expressed among Wilm's tumors, as well as other human cancers, including neuroblastoma, Ewing's sarcoma, bladder carcinomas, lung cancers, breast cancers, pancreatic cancers, esophageal cancers, and gastrointestinal cancers. In contrast, midkine is not expressed among marrow stem cells, mouse or human liver cells. It too has potential as a marker of minimal or occult disease.

GAGE1 and BAGE are cancer-testis antigens (CTA), which represent a large family of genes located on the X chromosome that become activated during tumorigenesis. They have near absolute tumor-specificity and are found only in testis and placenta, but no other normal tissues. As antigens, they are often thought to be “ignored” by the immune system. In addition, they are intracellular antigens and thus not easily available for antigen processing by antigen-presenting cells (APC) or attack by humoral antibodies. GAGE1 and BAGE are CTA with low frequency HLA motifs and they may prevail and be exploitable as markers of MRD. GAGE1 gene expression has been detected in various adult cancers including melanoma, non-small cell lung cancer, sarcoma, bladder cancers, as well as head and neck tumors. In neuroblastoma, expression of GAGE1 and BAGE was found in tumors of all stages, and absent in normal marrow and blood.

Using neuroblastoma as a test of principle, we had carried out microarray analysis of a large panel of human neuroblastomas (stages 2, 3, 4 and 4s) as well as ganglioneuromas using the Hu-95A Affymetrix microarray gene chip. We found that WT-1, midkine and GAGE1 were uniformly and highly expressed among all neuroblastomas, irrespective of stage, at substantial levels when compared to GD2 synthase, a marker we have already found to have prognostic significance in the study of minimal residual disease.

FAM-AGA AAA ACC GCT CTG GCC GCC AT-TAMRA
(BAGE PROBE)
5′-GAT GGT GGT GGC AAC AGA GA-3′
(BAGE FOR)
5′-TTC ATC AGC CTG GCT TGG A-3′
(BAGE REV)
FAM-CTC GTA CCC TGT GCT GTG GCC CTT T-TAMRA
(WT-1 PROBE)
5′-TCC CAG CTT GAA TGC ATG AC-3′
(WT-1 FOR)
5′-GAT GGG CGT TGT GTG GTT ATC-3′
(WT-1 REV)
FAM-TTG CCC TTC ACA TGC CAC AGA TGA TAG G-TAMRA
(GACE1 PROBE)
5′-CAA ATG GCG AGA GAC CGT TT-3′
(GAGE1 FOR)
5′-CAT AGG AGC AGC CTG CAA CA-3′
(GAGE1 REV)
FAM-TTC CCT TTC TTG GCT TTG GCC TTT GC-TAMRA
(MIDKINE PROBE)
5′-GCT CAG TGC CAG GAG ACC AT-3′
(MIDKINE FOR)
5′-TCC AGG CTT GGC GTC TAG TC-3′
(MIDKINE REV)

The above gene sequences are published and may be found by going to http://www.ncbi.nlm.nih.gov/entrez/guery.fcgi, searcing under Nucleotide, and entering the following accession numbers:

WT-1 NM_{—}000378

Midkine nm_{—}002391

GAGE1 nm_{—}001468

BAGE nm_{—}001187

What is claimed is:

1. A method to measure GD2 synthase mRNA comprising steps of:
a. obtaining an mRNA sample;
b. performing real-time quantitative RT-PCR on the sample using appropriate primers of GD2 synthase; and
c. determining the amount of GD2 mRNA.

2. The method of claim 1, where the sample is from a subject.

3. The method of claim 2, wherein the subject bears a cancer.

4. The method of claim 3, wherein the cancer is a neuroblastoma.

5. The method of claim 1, wherein, in step (b), the appropriate primers comprises a pair of sense and antisense primers.

6. The method of claim 5, wherein the sense primer is 5′ GACAA GCCAG AGCGC GTTA.

7. The method of claim 5, wherein the antisense primer is 5′-TACTT GAGAC ACGGC CAGGT T-3′.

8. The method of claim 1, wherein the real-time quantitative RT-PCR is performed with a probe.

9. The method of claim 8, wherein the probe is FAM-5′AACCA GCCCT TGCCG AAGGG C-3′

10. The method of claim 3, wherein the cancer is neuroblastoma, B cell lymphoma, small cell lung cancer, melanoma, osteosarcoma, soft tissue sarcoma, medulloblastoma, high-grade astrocytoma, or retinoblastoma.

11. A method to diagnose a subject which bears cancer expressing GD2 synthase comprising steps of:
a. obtaining a mRNA sample from the subject; performing RT-PCR on the sample using appropriate primers of GD2 synthase; and
b. determining the amount of GD2 mRNA.

12. The method of claim 11, wherein the appropriate primers are sense primer 5′-CCAACTCAACAGGCAACTAC-3′ and antisense primer 5′-GATCATAACGGAGGAAGGTC-3′.

13. The method of claim 11, wherein in step (b), the RT-PCR is a real-time quantitative RT-PCR.

14. The method of claim 13, wherein the appropriate primers comprises a pair of sense and antisense primers.

15. The method of claim 14, wherein the sense primer is 5′ GACAA GCCAG AGCGC GTTA.

16. The method of claim 14, wherein the antisense primer is 5′-TACTT GAGAC ACGGC CAGGT T-3′.

17. The method of claim 13, wherein the real-time quantitative RT-PCR is performed with a probe.

18. The method of claim 13, wherein the probe is FAM-5′AACCA GCCCT TGCCG AAGGG C-3′.

19. The method of claim 11, wherein the cancer is neuroblastoma, B cell lymphoma, small cell lung cancer, melanoma, osteosarcoma, soft tissue sarcoma, medulloblastoma, high-grade astrocytoma, or retinoblastoma.

20. A method to stage a cancer expressing GD2 synthase in a subject comprising steps of:
a. obtaining a mRNA sample from the subject known to carry the disease at different stages;
b. performing real-time quantitative RT-PCR on the sample obtained from step (a) using appropriate primers of GD2 synthase;
c. determining the amount of the GD2 mRNA;
d. comparing the amount obtained in step (c) from the different stages to obtain a standard curve;
e. obtaining a mRNA sample from a test subject with the cancer;
f. determining the amount of mRNA from the sample using real-time quantitative RT-PCR; and
g. comparing the amount determined in step (f) with the standard curve obtained in step (d), thereby determining the stage of the cancer in the test subject.

21. The method of claim 20, wherein the cancer is neuroblastoma, B cell lymphoma, small cell lung cancer, melanoma, osteosarcoma, soft tissue sarcoma, medulloblastoma, high-grade astrocytoma, or retinoblastoma.

22. The method of claim 20, wherein the appropriate primers comprises a pair of sense and antisense primers.

23. The method of claim 22, wherein the sense primer is 5′ GACAA GCCAG AGCGC GTTA.

24. The method of claim 22, wherein the antisense primer is 5′-TACTT GAGAC ACGGC CAGGT T-3′.

25. The method of claim 22, wherein the real-time quantitative RT-PCR is performed with a probe.

26. The method of claim 20, wherein the probe is FAM-5′AACCA GCCCT TGCCG AAAGGG C-3′.

27. A method for determining tumor cells in a sample comprising steps of: a) obtaining a mRNA sample; b) performing real-time quantitative RT-PCR on the sample using appropriate primers of GD2 synthase; and c) determining the amount of GD2 mRNA, thereby determining the tumor cells in the sample.

28. The method of claim 27, wherein, in step (b), the appropriate primers comprises a pair of sense and antisense primers.

29. The method of claim 28, wherein the sense primer is 5′ GACAA GCCAG AGCGC GTTA.

30. The method of claim 28, wherein the antisense primer is 5′-TACTT GAGAC ACGGC CAGGT T-3′.

31. The method of claim 27, wherein the real-time quantitative RT-PCR is performed with a probe.

32. The method of claim 31, wherein the probe is FAM-5′AACCA GCCCT TGCCG AAGGG C-3′

33. The method of claim 27, wherein the cancer is neuroblastoma, B cell lymphoma, small cell lung cancer, melanoma, osteosarcoma, soft tissue sarcoma, medulloblastoma, high-grade astrocytoma, or retinoblastoma.

34. A method for determining the minimum residual disease of a cancer comprising steps of: a) obtaining a mRNA sample; b) performing real-time quantitative RT-PCR on the sample using appropriate primers of GD2 synthase; and c) determining the amount of GD2 mRNA, the positive expression of GD2 indicating the residual disease.

35. The method of claim 34, wherein, in step (b), the appropriate primers comprises a pair of sense and antisense primers.

36. The method of claim 35, wherein the sense primer is 5′ GACAA GCCAG AGCGC GTTA.

37. The method of claim 35, wherein the antisense primer is 5′-TACTT GAGAC ACGGC CAGGT T-3′.

38. The method of claim 34, wherein the real-time quantitative RT-PCR is performed with a probe.

39. The method of claim 38, wherein the probe is FAM-5′AACCA GCCCT TGCCG AAGGG C-3′

40. The method of claim 34, wherein the cancer is neuroblastoma, B cell lymphoma, small cell lung cancer, melanoma, osteosarcoma, soft tissue sarcoma, medulloblastoma, high-grade astrocytoma, or retinoblastoma.

41. The method of claim 27 or 34 further comprising determining the level of expression of GAGE1.

42. A kit for detection of GD2 synthase comprising a compartment containing 5′ GACAA GCCAG AGCGC GTTA; and another compartment containing 5′-TACTT GAGAC ACGGC CAGGT T-3′.

43. The kit for claim 42 further comprising FAM-5′AACCA GCCCT TGCCG AAGGG C-3′.

44. A method for determining the minimum residual disease of a cancer comprising steps of: a) obtaining a mRNA sample; b) performing real-time quantitative RT-PCR on the sample using appropriate primers of a marker; and
c) determining the amount of the marker's mRNA, the positive expression of the marker indicating the residual disease.

45. The method of claim 44 wherein the marker is WT-1, Midkine, GAGE1 or BAGE.

46. The method of claim 44, wherein, in step (b), the appropriate primers comprises a pair of sense and antisense primers.

47. The method of claim 46, wherein the sense primer is 5′-TCC CAG CTT GAA TGC ATG AC-3′.

48. The method of claim 46, wherein the antisense primer is 5′-GAT GGG CGT TGT GTG GTT ATC-3′.

49. The method of claim 46, wherein the real-time quantitative RT-PCR is performed with a probe.

50. The method of claim 46, wherein the probe is FAM-CTC GTA CCC TGT GCT GTG GCC CTT T-TAMRA.

51. The method of claim 46, wherein the cancer is Wilm's tumors, neuroblastoma, Ewing's sarcoma, bladder carcinomas, lung cancers, breast cancers, pancreatic cancers, esophageal cancers, or gastrointestinal cancers.

52. The method of claim 46, wherein the sense primer is 5′-GCT CAG TGC CAG GAG ACC AT-3′.

53. The method of claim 46, wherein the antisense primer is 5′-TCC AGG CTT GGC GTC TAG TC-3′.

54. The method of claim 46, wherein the real-time quantitative RT-PCR is performed with a probe.

55. The method of claim 46, wherein the probe is FAM-TTC CCT TTC TTG GCT TTG GCC TTT GC-TAMRA.

56. The method of claim 46, wherein the sense primer is 5′-CAA ATG GCG AGA GAC CGT TT-3′.

57. The method of claim 46, wherein the antisense primer is 5′-CAT AGG AGC AGC CTG CAA CA-3′.

58. The method of claim 46, wherein the real-time quantitative RT-PCR is performed with a probe.

59. The method of claim 46, wherein the probe is FAM-TTG CCC TTC ACA TGC CAC AGA TGA TAG G-TAMRA.

60. The method of claim 46, wherein the cancer is melanoma, non-small cell lung cancer, sarcoma, bladder cancers, head and neck tumors, or neuroblastoma.

61. The method of claim 46, wherein the sense primer is 5′-GAT GGT GGT GGC AAC AGA GA-3′.

62. The method of claim 46, wherein the antisense primer is 5′-TTC ATC AGC CTG GCT TGG A-3′.

63. The method of claim 46, wherein the real-time quantitative RT-PCR is performed with a probe.

64. The method of claim 46, wherein the probe is FAM-AGA AAA ACC GCT CTG GCC GCC AT-TAMRA.

65. The method of claim 46, wherein the cancer is melanoma, non-small cell lung cancer, sarcoma, bladder cancers, head and neck tumors, or neuroblastoma.

66. A method for determining tumor cells in a sample comprising steps of: a) obtaining a mRNA sample; b) performing real-time quantitative RT-PCR on the sample using appropriate primers of a marker; and c) determining the amount of markers mRNA, thereby determining the tumor cells in the sample.

67. The method of claim 66, wherein, in step (b), the appropriate primers comprises a pair of sense and antisense primers.

68. The method of claim 67 wherein the marker is WT-1, Midkine, GAGE1 or BAGE.

69. The method of claim 66, wherein the sense primer is 5′-TCC CAG CTT GAA TGC ATG AC-3′.

70. The method of claim 66, wherein the antisense primer is 5′-GAT GGG CGT TGT GTG GTT ATC-3′.

71. The method of claim 66, wherein the real-time quantitative RT-PCR is performed with a probe.

72. The method of claim 66, wherein the probe is FAM-CTC GTA CCC TGT GCT GTG GCC CTT T-TAMRA.

73. The method of claim 66, wherein the cancer is Wilm's tumors, neuroblastoma, Ewing's sarcoma, bladder carcinomas, lung cancers, breast cancers, pancreatic cancers, esophageal cancers, or gastrointestinal cancers.

74. The method of claim 66, wherein the sense primer is 5′-GCT CAG TGC CAG GAG ACC AT-3′.

75. The method of claim 66, wherein the antisense primer is 5′-TCC AGG CTT GGC GTC TAG TC-3′.

76. The method of claim 66, wherein the real-time quantitative RT-PCR is performed with a probe.

77. The method of claim 66, wherein the probe is FAM-TTC CCT TTC TTG GCT TTG GCC TTT GC-TAMRA.

78. The method of claim 66, wherein the sense primer is 5′-CAA ATG GCG AGA GAC CGT TT-3′.

79. The method of claim 66, wherein the antisense primer is 5′CAT AGG AGC AGC CTG CAA CA-3′.

80. The method of claim 66, wherein the real-time quantitative RT-PCR is performed with a probe.

81. The method of claim 66, wherein the probe is FAM-TTG CCC TTC ACA TGC CAC AGA TGA TAG G-TAMRA.

82. The method of claim 66, wherein the cancer is melanoma, non-small cell lung cancer, sarcoma, bladder cancers, head and neck tumors, or neuroblastoma.

83. The method of claim 66, wherein the sense primer is 5′-GAT GGT GGT GGC AAC AGA GA-3′.

84. The method of claim 66, wherein the antisense primer is 5′-TTC ATC AGC CTG GCT TGG A-3′.

85. The method of claim 66, wherein the real-time quantitative RT-PCR is performed with a probe.

86. The method of claim 66, wherein the probe is FAM-AGA AAA ACC GCT CTG GCC GCC AT-TAMRA.

87. The method of claim 66, wherein the cancer is melanoma, non-small cell lung cancer, sarcoma, bladder cancers, head and neck tumors, or neuroblastoma.

88. A kit for detection of WT-1 comprising a compartment containing 5′-TCC CAG CTT GAA TGC ATG AC-3′; and another compartment containing 5′-GAT GGG CGT TGT GTG GTT ATC-3′.

89. The kit for claim 88 further comprising FAM-CTC GTA CCC TGT GCT GTG GCC CTT T-TAMRA.

90. A kit for detection of Midkine comprising a compartment containing 5′-GCT CAG TGC CAG GAG ACC AT-3′; and another compartment containing 5′-TCC AGG CTT GGC GTC TAG TC-3′.

91. The kit for claim 90 further comprising FAM-TTC CCT TTC TTG GCT TTG GCC TTT GC-TAMRA.

92. A kit for detection of GAGE1 comprising a compartment containing 5′-CAA ATG GCG AGA GAC CGT TT-3′; and another compartment containing 5′-CAT AGG AGC AGC CTG CAA CA-3′.

93. The kit for claim 92 further comprising FAM-TTG CCC TTC ACA TGC CAC AGA TGA TAG G-TAMRA.

94. A kit for detection of RAGE comprising a compartment containing 5′-GAT GGT GGT GGC AAC AGA GA-3′; and another compartment containing 5′-TTC ATC AGC CTG GCT TGG A-3′.

95. The kit for claim 94 further comprising FAM-AGA AAA ACC GCT CTG GCC GCC AT-TAMRA.

96. A method to measure mRNA of WT-1, Midkine, GAGE1, or BAGE comprising steps of:
a. obtaining an mRNA sample;
b. performing real-time quantitative RT-PCR on the sample using appropriate primers of WT-1, Midkine, GAGE1, or BAGE; and
c. determining the amount of WT-1, Midkine, GAGE1, or BAGE.

97. A method to diagnose a subject which bears cancer expressing WT-1, Midkine, GAGE1, or BAGE, comprising steps of:
a. obtaining a mRNA sample from the subject; performing RT-PCR on the sample using appropriate primers of WT-1, Midkine, GAGE1, or BAGE; and
b. determining the amount of mRNA of WT-1, Midkine, GAGE1, or BAGE.
